(19) United States
(12) Patent Application Publication
(10) Pub. No.: US 2010/0034812 A1
(43) Pub. Date: Feb. 11, 2010

(54) Antibodies Specific for BET V1 and Use Thereof in the Prevention and Treatment of BET V1- Induced Diseases

(75) Inventors: Otto Majdic, Vienna (AT); Petra Kohl, Vienna (AT); Rudolf Valenta, Theresienfeld (AT); Sabine Flicker, Vienna (AT); Katharina Marth, Vienna (AT); Anna Gieras, Vienna (AT)

Correspondence Address: CHALKER FLORES, LLP, 2711 LBJ FRWY, Suite 1036, DALLAS, TX, 75234, US

(73) Assignee: Biomay AG, Vienna (AT)

(21) Appl. No.: 12/301,219

(22) PCT Filed: May 18, 2007
(86) PCT No.: PCT/AT07/00238

§ 371 Date: Nov. 17, 2008

(30) Foreign Application Priority Data
May 18, 2006 (AT) A 866/2006

(51) Int. Cl. A61K 39/395; C07H 21/04; C07K 16/00; A61P 37/00

(52) U.S. Cl. 424/133.1; 424/139.1; 536/23.1; 530/387.1

(57) ABSTRACT

The present invention relates to Bet V 1 specific antibodies or fragments thereof and their use in the prevention and treatment of allergen induced diseases, wherein the antibodies block the binding of IgE to Bet V 1.

The present invention relates to antibodies and pharmaceutical formulations for the treatment and prevention of allergen induced diseases.

Almost 100 million allergic patients are sensitized to the major birch (Betula verrucosa) pollen allergen, Bet v 1, a 17 kDa protein, which is present in pollens of trees belonging to the Fagales order and is widely distributed in Europe, North America, Russia and Australia (Breiteneder et al., 1989). The cDNA coding for Bet v 1 has been isolated (Breiteneder et al., 1989) and recombinant Bet v 1, which equals the natural Bet v 1 wild-type, was expressed in Escherichia coli (Valenta et al., 1991; Ferreira et al., 1993). The recognition of Bet v 1 by IgE antibodies of patients allergic to tree pollen and food averages about 95% and almost 60% of them are sensitized exclusively against Bet v 1 (Jarolim et al., 1989), whereby the recognition of the allergen depends on conformational epitopes and hence requires a folded molecule. Because of the previous extensive in vitro and in vivo characterization, the recombinant Bet v 1 molecule has often been proposed to be used for diagnostic and therapeutic purposes (Valenta et al., 1995, 1996). Recently non-anaphylactic surface-exposed peptides of the major birch pollen allergen were generated and characterized as peptides with a size (25-32 amino acids) sufficient to induce antibody responses in vivo (active immunisation). These peptides lacked fold and allergenic activity. However, peptide vaccination induced the production of polyclonal Bet v 1 specific IgG (Focke et al., 2004).

WO 94/10194 relates to peptides derived from trees of the Fagales order.

In EP 1 219 300 the use of allergen derivatives for the manufacture of medicaments for the treatment of allergies is described.

It is an object of the present invention to provide means and methods for the treatment and the prevention of allergic reactions caused by the birch pollen allergen Bet v 1 or fragments thereof.

Therefore, the present invention relates to the use of an antibody or derivative thereof for the manufacture of a medicament for the passive immunization of an individual for the prevention and/or treatment of allergic reactions in said individual caused by an exposure to a birch pollen allergen, wherein the antibody binds to a Bet v 1 fragment comprising amino acids 30 to 59 (SEQ ID No. 1) or amino acids 75 to 104 (SEQ ID No. 2).

It surprisingly turned out that antibodies or derivatives thereof binding to said Bet v 1 fragments are able to bind specifically to the birch pollen allergen Bet v 1 and that such molecules may be used to block the binding of Bet v 1 specific IgE to said birch pollen allergens. The binding of the antibodies according to the present invention to epitopes within or closely-related to the major IgE binding sites of Bet v 1 and/or the modification of the conformation of the allergen, so that the IgE epitopes or just a part of them are not longer accessible for IgE, result in a reduced or even complete reduction of the binding of IgE to said allergen. Experimental data show that a mixture of two peptide-specific antibodies, with a different epitope-specificity did not yield a stronger inhibition of IgE binding than the individual antibodies demonstrating that one antibody alters the conformation of the allergen inhibiting the binding of a second antibody (also IgE) to said allergen.

For the production of antibodies, various host animals may be immunized by injection with the Bet v 1 antigen or fragments thereof, in particular with Bet v 1 fragments consisting of amino acids 30 to 59 (SEQ ID No. 1) or amino acids 75 to 104 (SEQ ID No. 2). Such host animals may include e.g. pigs, rabbits, mice, goats, and rats. Most preferably the polyclonal antibodies are isolated from a human individual. The use of such antibodies reduces the risk that the immune system will respond to “foreign” antibody derived antigens. The antibodies may be isolated from the sera of these animals.

Antibodies according to the present invention may be formulated for intravenous, intramuscular, sub cutaneous and local administration protocols for obtaining such formulations are known to the skilled artisan.

Of course it is also possible to isolate antibodies directed to the Bet v 1 fragments according to the present invention from human individuals exposed to the Bet v 1 allergen. The isolation of Bet v 1 specific antibodies from human individuals can be achieved by methods known in the art.

As used herein, “antibodies” refer to intact immunoglobulins or to fragments thereof produced, for instance, by digestion with various peptidases or recombinantly. Of course, also molecules comprising the antigen binding region of immunoglobulins fused to other proteins or fragments thereof are intended to be antibodies according to the present invention. “The antigen binding region” refers to the part of an immunoglobulin molecule that participates in antigen binding. The antigen binding region is formed by amino acid residues of the N-terminal variable regions of the heavy and light chains. Therefore the term “antibodies” refers, but is not limited to, to Fab's (e.g. produced by pepsin digestion of an antibody below the disulfide linkages in the hinge region or produced by recombinant methods), single chain antibodies (antibodies that exist as a single polypeptide chain), more preferably single chain Fv antibodies (scFv) in which a variable heavy and a variable light chain are joined together (directly or through a peptide linker) to form a continuous polypeptide, chimeric molecules, humanized molecules etc.

The antibodies of the invention include also derivatives that are modified chemically, by recombinant DNA technology, enzymatically etc. resulting in e.g. “technically modified antibodies” such as synthetic antibodies, chimeric or humanized antibodies, or mixtures thereof, or antibody fragments which partially or completely lack the constant region, e. F. Ev, Fab, Fab′ or F(ab)′2 etc. In these technically modified antibodies, e.g., a part or parts of the light and/or heavy chain may be substituted. Such molecules may, e.g., comprise antibodies consisting of a humanized heavy chain and an unmodified light chain (or chimeric light chain), or vice versa. The terms Fv, Fc, Fd, Fab, Fab′ or F (ab) 2 are used as described in the prior art (Harlow E. and Lane D., in “Antibodies, A Laboratory Manual”, Cold Spring Harbor Laboratory, 1988). “Derivatives” of antibodies in this context refers to proteinaceous molecules comprising one or more functional activities associated with a full-length antibody according to the present invention. Thus, the antibody derivatives according to the present invention are able to bind to a Bet v 1 fragment comprising amino acids 30 to 59 (SEQ ID No. 1) or amino acids 75 to 104 (SEQ ID No. 2). The antibodies of the invention include also derivatives that are modified, i.e., by the covalent attachment of any type or molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids. Derivatives according to the present invention may also comprise fragments which still are able to bind a Bet v 1 fragment comprising amino acids 30 to 59 (SEQ ID No. 1) or amino acids 75 to 104 (SEQ ID No. 2) (e.g. CDR region of an antibody according to the present invention).

The antibodies according to the present invention are preferably monoclonal antibodies. Such antibodies, which are homogeneous populations of antibodies to a particular antigen, can be obtained by any technique which provides for the production of antibody molecules by continuous cell lines in culture. These include, for instance, the hybridoma technique of Köhler and Milstein (1975, Nature 256:495-497 and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030) and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production. Of course it is also possible to produce monoclonal antibodies by recombinant technologies in eukaryotic, yeast, insect and plant cells and in plants. These expression systems as well as methods for the isolation of said antibodies from said cells are well known in the art. It was very surprising that a monoclonal antibody directed to the Bet v 1 fragments according to the present invention exhibits an inhibition similar to polyclonal antisera which are produced by expressing a mammal to the entire Bet v 1 allergen (s. e.g. Focke et al., 2004). Especially taking in consideration that polyclonal antibodies are normally directed to more than one epitope. The production of monoclonal antibodies leads to a product which is much more homogeneous and pure and hence reproducible than polyclonal antibodies obtained from antisera.

The selection of suitable peptides for the production of monoclonal antibodies which may be used to treat and/or prevent allergic disease caused by the exposure to Bet v 1 is not trivial. Lebeque et al. (1997), for instance, investigated the effects of various monoclonal antibodies raised against Bet v 1 on the binding to Bet v 1 of IgE from patients allergic to said allergen. These studies revealed, without disclosing the specificity of the antibodies produced to regions of the Bet v 1 allergen, that certain monoclonal antibodies enhance IgE binding to Bet v 1 rather than reduce said binding. Therefore it was surprising that the antibodies according to the present invention strongly inhibit IgE binding to wild-type Bet v 1.

The antibody according to the present invention and a vaccine formulation comprising said antibody may be used not only to treat allergic reactions caused by the birch pollen allergen Bet v 1, but also to prevent such reactions or to sensitize an individual for the Bet v 1 allergen. It is also possible to vaccinate a child or newborn with an antibody or vaccine formulation according to the present invention before said child or newborn will get in contact with birch pollen. Such an approach will prevent the formation of Bet v 1 specific IgE antibodies and thus sensibilisation to Bet v 1 in said child or newborn. It is particular advantageous to administer the antibodies according to the present invention to children within the age of 1 to 3 because at this age children get sensibilised to birch pollen allergens.

According to a preferred embodiment of the present invention the antibody is an IgG antibody, in particular an IgG antibody of the IgG1 or IgG4 isotype.

Sellge et al. (Clin. Exp. Allergy (2005) 35: 774-781) showed that IgG antibodies directed to Bet v 1 enhance allergic reactions by the formation of larger allergen aggregates activating most cells or basophils. Similar results more obtained by the experiments disclosed in Laffer et al. (J Immunol (1996) 157: 4953-4962) and Denepoux et al. (FEBS letters (2000) 465: 39-46). However, the use of IgG antibodies directed to Bet v 1 fragments according to the present invention did not show these effects. Therefore IgG antibodies may be used according to the present invention.

More preferred antibodies according to the present invention are non-complement activating antibodies like human IgG4 or murine IgG1.

The antibody according to the present invention is preferably a murine or human antibody.

According to another preferred embodiment of the present invention the antibody is a chimeric antibody.

Techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, immunoglobulin classes, subclasses (isotypes), types and subtypes, e.g. as those having a variable region derived from a murine mAb and a human immunoglobulin constant region. Furthermore, chimeric antibodies according to the present invention may comprise more than one specificity (e.g. diabodies or tetrabodies).

The antibody according to the present invention is preferably humanized.

Methods for “humanizing” antibodies, or generating less immunogenic fragments of non-human antibodies, are well known. A humanized antibody is one in which only the antigen-recognized sites, or complementary-determining hypervariable regions (CDRs) are of non-human origin, whereas all framework regions (FR) of variable domains are products of human genes.

Non-human antibodies may be humanized by any of the methods known in the art. In one method, the non-human CDRs are inserted into a human antibody or consensus antibody framework sequence. Further changes can then be introduced into the antibody framework to modulate affinity or immunogenicity. Following are protocols to improve the monoclonal antibodies directed to fragments of the birch pollen allergen Bet v 1 as therapeutics in humans by “humanizing” the monoclonal antibodies to improve their serum half-life and render them less immunogenic in human hosts (i.e. to prevent human antibody response to non-human antibodies). The principles of humanization have been described in the literature and are facilitated by the modular arrangement of antibody proteins. To minimize the possibility of binding complement, a humanized antibody of the IgG1 isotype is preferred. For example, a level of humanization is achieved by generating chimeric antibodies comprising the variable domains of non-human antibody proteins of interest with the constant domains of human antibody molecules (e.g. Morrison et al., Adv. Immunol., 1989, 44, 65-92). The variable domains of Bet v 1 specific antibodies may be cloned from the cDNA generated from mRNA isolated from the hybridoma of interest. The variable region gene fragments are linked to exons encoding human antibody constant domains, and the resultant construct is expressed in suitable mammalian host cells (e.g. myeloma or CHO cells). To achieve an even greater level of humanization, only those portions of the variable region gene fragments that encode antigen-binding complementarity determining regions (“CDR”) of the non-human monoclonal antibody genes may be cloned into human antibody sequences (e.g. Jones et al., Nature, 1986, 321, 522-525, Riechmann et al., Nature, 1988, 332, 323-327, verhoeyen et al, Science, 1988, 239, 1534-36, and Tempest et al., Bio/Technology, 1991, 9, 266-71). Also the beta-sheet framework of the human antibody surrounding the CDR3 regions may be modified to more closely mirror the three dimensional structure of the antigen-binding domain of the original monoclonal antibody (see Kettleborough et al., Protein Engin., 1991, 4, 773-783, and Foote et al., J. Mol. Biol., 1992, 224, 487-499). In an alternative approach, the surface of a non-human monoclonal antibody of interest is humanized by altering selected surface residues of the non-human antibody, e.g. by site-directed mutagenesis, while retaining all of the interior and contacting residues of the non-human antibody (Padlan, Molecular Immunol, 1991, 28, 489-98).

Another aspect of the present invention relates an antibody or fragment thereof binding to a fragment of Bet v 1, characterized in that said fragment of Bet v 1 consists of amino acids 30 to 59 (SEQ ID No. 1) or amino acids 75 to 104 (SEQ ID No. 2).

The antibody according to the present invention is preferably a monoclonal antibody secreted by a hybridoma deposited under the Budapest Treaty with the DSMZ (Deutsche Sammlung von Mikroorganismen und Zellkulturen, Braunschweig, Germany) on 9 May 2006 and assigned accession numbers DSM ACC2782, DSM ACC2783, DSM ACC2785, DSM ACC2784 and DSM ACC2786.

Another aspect of the present invention relates to a vaccine formulation comprising an antibody according to the present invention.

The antibodies of the present invention may be formulated for administration to a mammal, in particular to a human, in a variety of ways. In some embodiments, the antibodies are in sterile aqueous solution or in biological fluids such as serum. Aqueous solutions may be buffered or unbuffered and have additional active or inactive components. Additional components include salts for modulating ionic strength, preservatives including, but not limited to, antimicrobials, anti-oxidants, chelating agents and the like, and nutrients including glucose, dextrose, vitamins and minerals. Alternatively, antibodies may be prepared for administration in solid form. The antibodies may be combined with a number of inert carriers or excipients, including but not limited to; binders such as microcrystalline cellulose, gum tragacanth or gelatin; excipients such as starch or lactose; dispersing agents such as alginic acid, Primogel, or corn starch; lubricants such as magnesium stearate; glidants such as colloidal silicon dioxide; sweetening agents such as sucrose or saccharin; or flavoring agents such as peppermint or methyl salicylate. Antibodies or their formulations may be administered to a mammal by any means effective for delivering the antibodies to the target. Such means include intravenous, intramuscular, subcutaneous, oral, intranasal, mucosal or dermal dosage forms. Localized administration of the antibodies or vaccine formulations according to the present invention is preferred. Phosphate buffered saline (PBS) is a preferred carrier for injectable formulations and for formulations which may be administered intranasal. Dosing of antibodies to obtain a pharmaceutically effective amount of therapeutic agent depends on a variety of factors. For example, age, sensitivity, tolerance, and other characteristics of the patient will affect dosing amounts. Furthermore, plasma level and half-life of the antibodies employed and affinity for their recognition sites, and other similar factors need to be considered for effective dosing. For systemic administration of the antibodies according to the present invention, doses ranging from about 1 mg/kg-patient/day to about 500 mg/kg-patient/day, preferably from about 5 mg/kg-patient/day to about 250 mg/kg-patient/day, more preferably from about 10 mg/kg-patient/day to about 100 mg/kg-patient/day, can be used, although dosages in the lower end of the range are preferred simply for ease of administration and cost effectiveness. Dosages may be adjusted, for example, to provide a particular plasma level of an antibody, e.g., in the range of about 0.05 to 200 μg/ml, more preferably of about 0.1 to 100 μg/ml, and to maintain that level, e.g., for a period of time or until clinical results are achieved. Chimeric and humanized antibodies, which would be expected to be cleared more slowly, would require lower dosages to maintain an effective plasma level. Also, antibodies having high affinity for the Bet v 1 fragments are preferably administered less frequently or in lower doses than antibodies with less affinity. A therapeutically effective dosage of antibody can be determined by showing, during the course of treatment, reduction of allergic reactions. Preferably the vaccine formulation and the medicament according to the present invention is administered to a individual up to one or two weeks prior the pollen season.

The vaccine formulation is preferably adapted for intramuscular, subcutaneous, intravenous or mucosal administration.

The formulation according to the present invention may be administered in various ways, whereby intramuscular, subcutaneous, intravenous or mucosal administration are preferred. The antibodies binding specifically to the Bet v 1 fragments consisting of amino acids 30 to 59 (SEQ ID No. 1) or amino acids 75 to 104 (SEQ ID No. 2) may be administered to an individual to treat or to prevent allergic reactions caused by birch pollen allergen Bet v 1. Especially mucosal administration of the antibodies according to the present invention would have a number of advantages over traditional immunization regimes. Paramount amongst these are more effective stimulation of the local mucosal immune system of the respiratory tract and the likelihood that vaccine uptake rates would be increased because the fear and discomfort associated with injections would be avoided. The use of antibodies which bind to allergens according to the present invention may help to combat the allergic reactions by inhibiting the binding of IgE to the allergens. As a result of this inhibition the IgE production, which upon contact with the allergen would normally increase, may be reduced.

Another aspect of the present invention relates to nucleic acid molecules comprising a nucleotide sequence selected from the group consisting of SEQ ID No. 3 to 298.

The nucleotide sequences SEQ ID No. 3 to 298 are derived from the mRNA encoding variable regions of IgE molecules which are able to bind solely to Bet v 1 and, hence, encode for polypeptides which bind to said allergen.

Another aspect of the present invention relates to a polypeptide encoded by a nucleic acid molecule comprising a nucleotide sequence selected from the group consisting of SEQ ID No. 3 to 298.

Yet another aspect of the present invention relates to an antibody or fragment thereof comprising a polypeptide encoded by a nucleic acid molecule comprising a nucleotide sequence selected from the group consisting of SEQ ID No. 3 to 298.

The polypeptide and the nucleic acid molecule of the present invention may be incorporated (e.g. by molecular biological methods) into an antibody or fragment thereof, so that said antibody or fragment thereof is also able to bind solely to Bet v 1. Such antibodies or fragments may be used, for instance, for passive immunization against Bet v 1.

According to a preferred embodiment of the present invention the antibody is an immunoglobulin selected from the group consisting of IgG1, IgG2, IgG3 and IgG4.

According to another preferred embodiment of the present invention the fragment is a constant region of an immunoglobulin, a variable region of an immunoglobulin, single chain Fv (scFv), diabodies (dsFv), Fab or combinations thereof.

The present invention is further illustrated by the following examples and figures, without being restricted thereto.

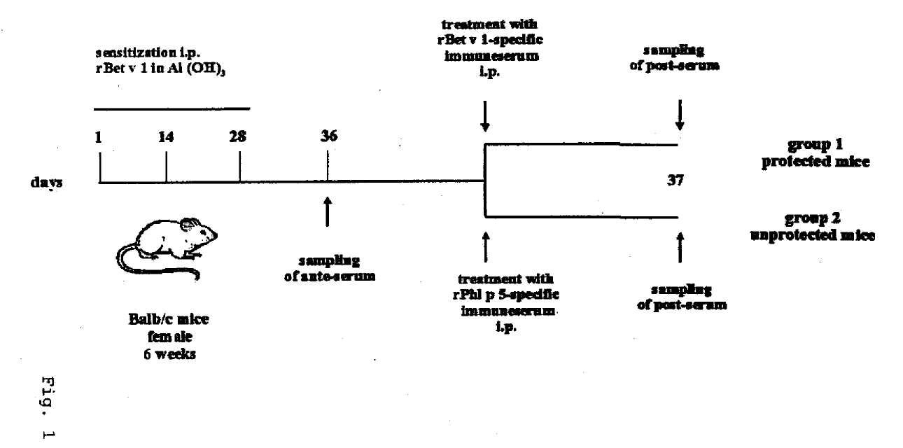

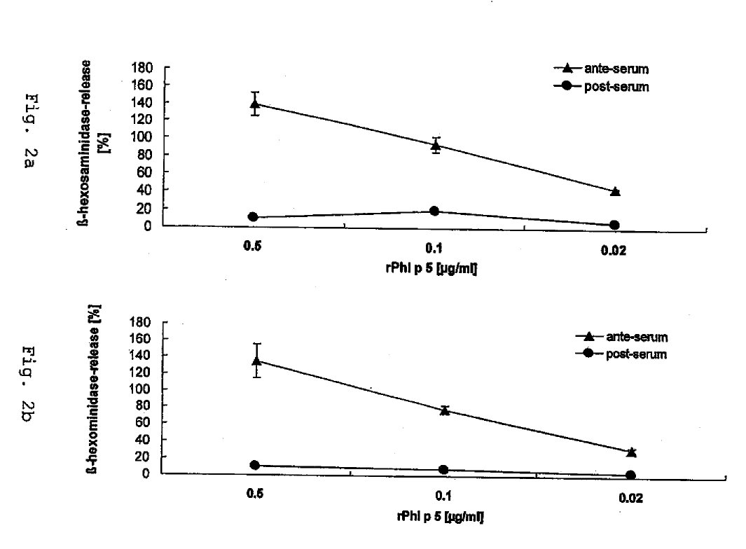

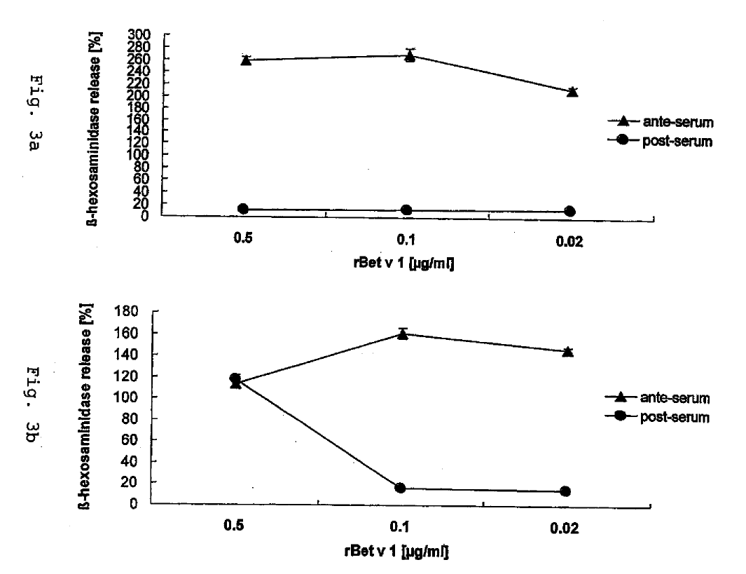

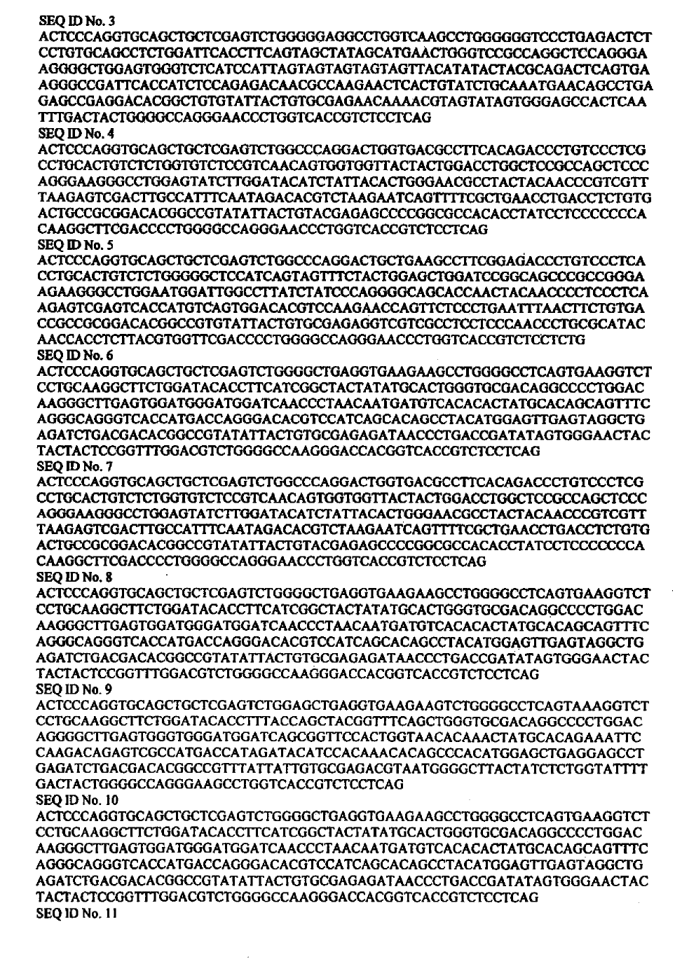

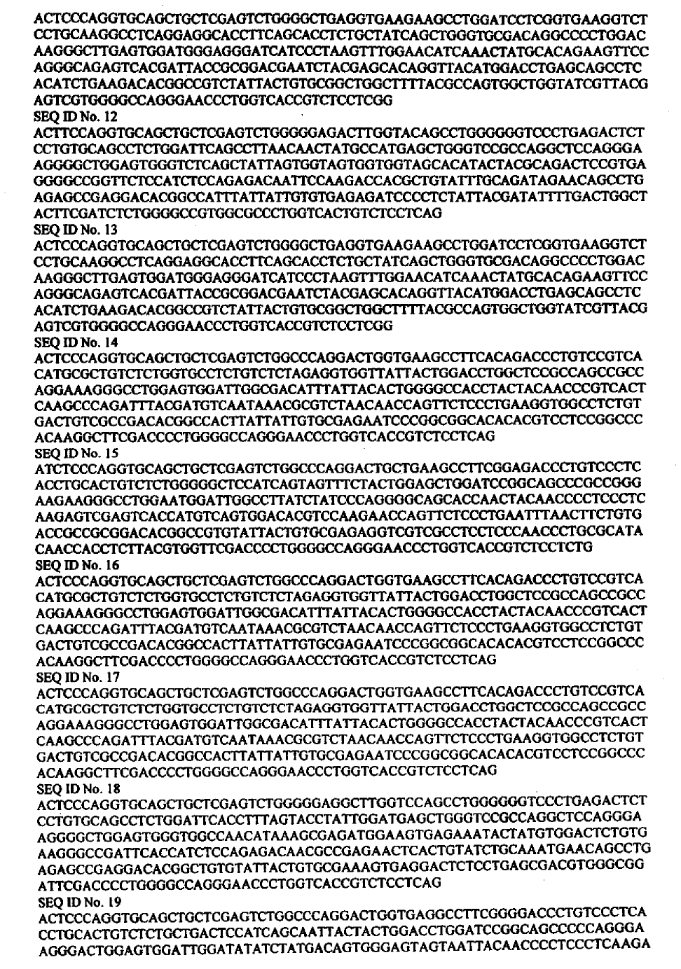

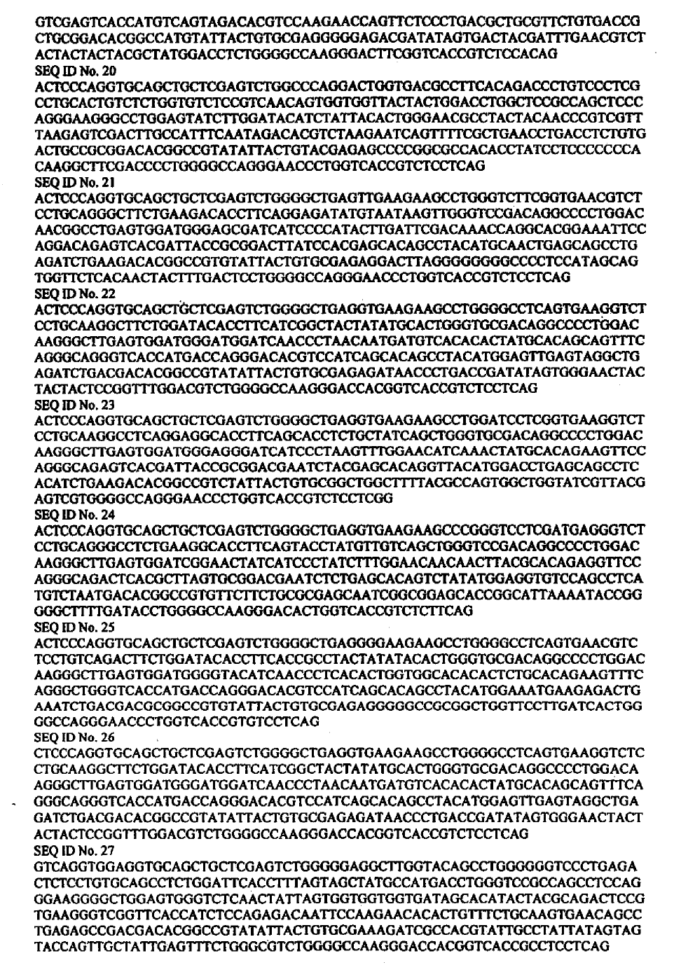

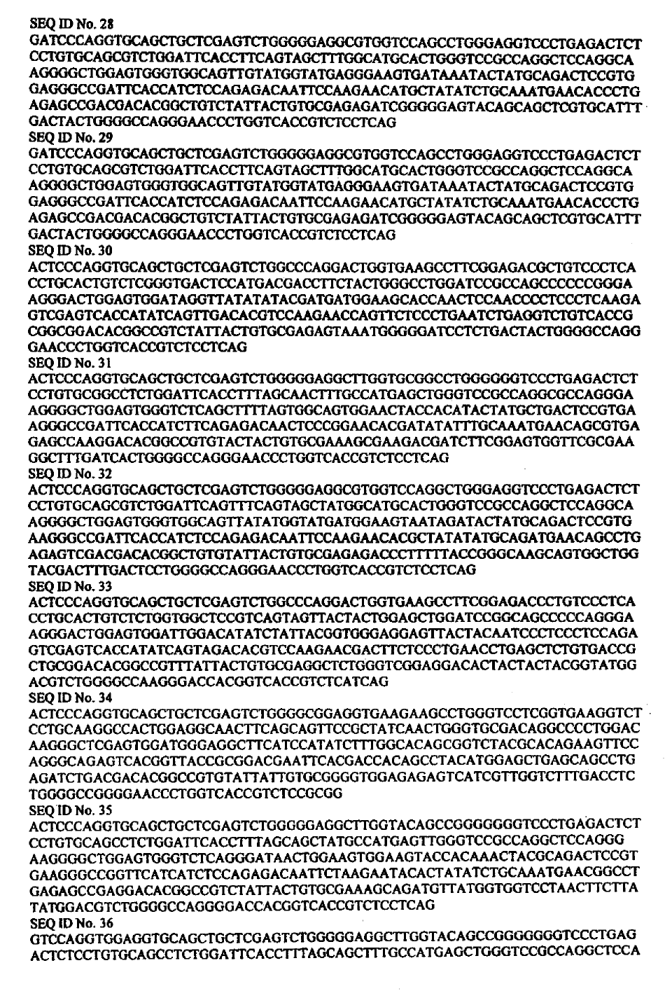

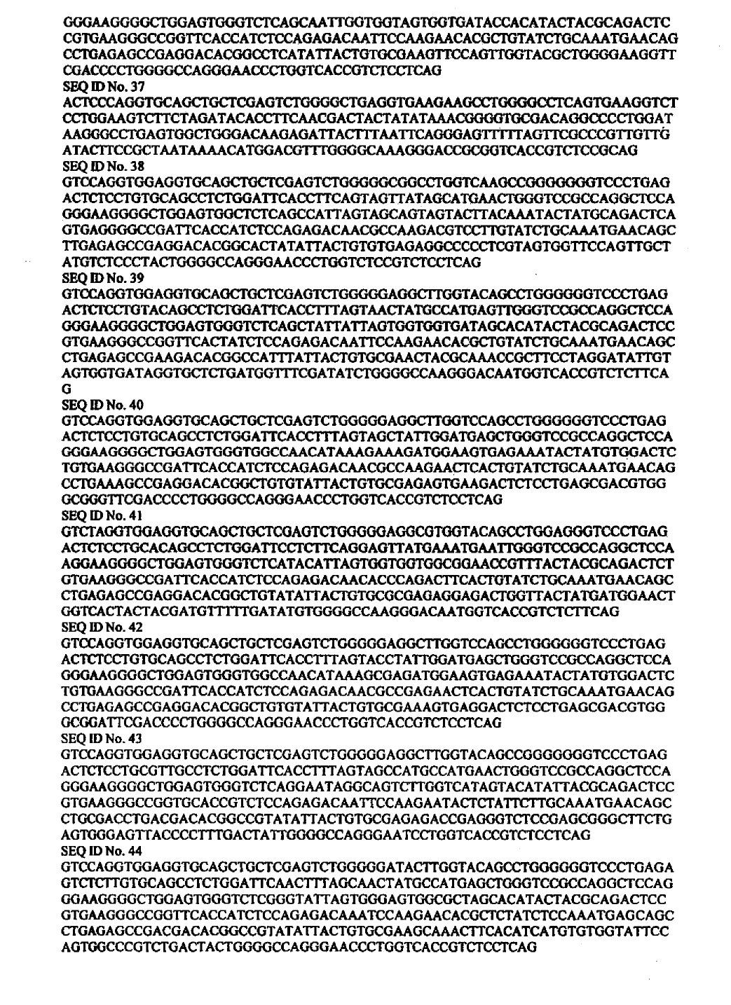

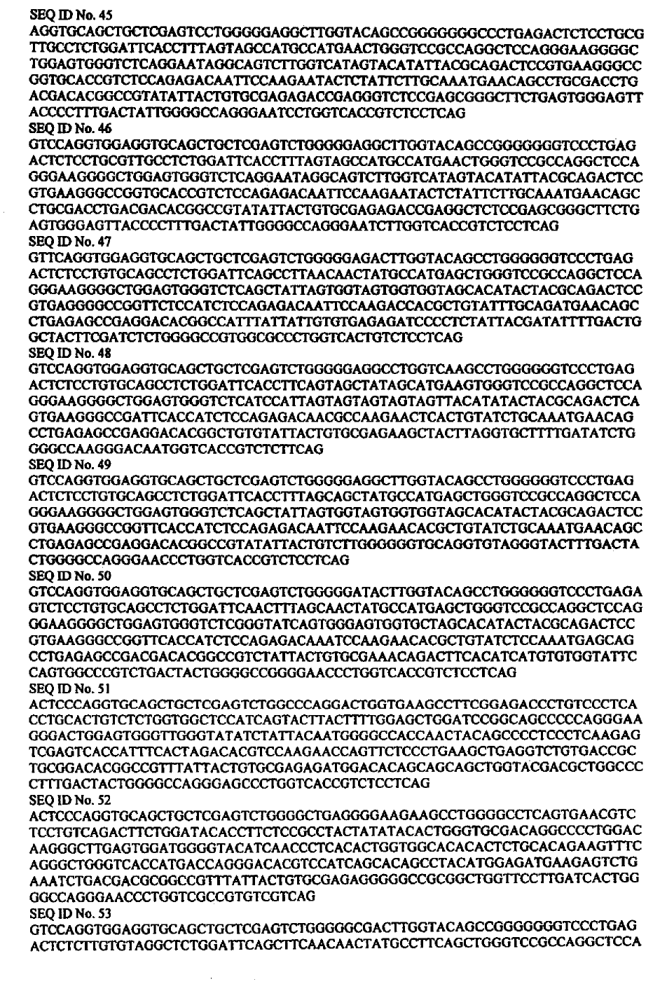

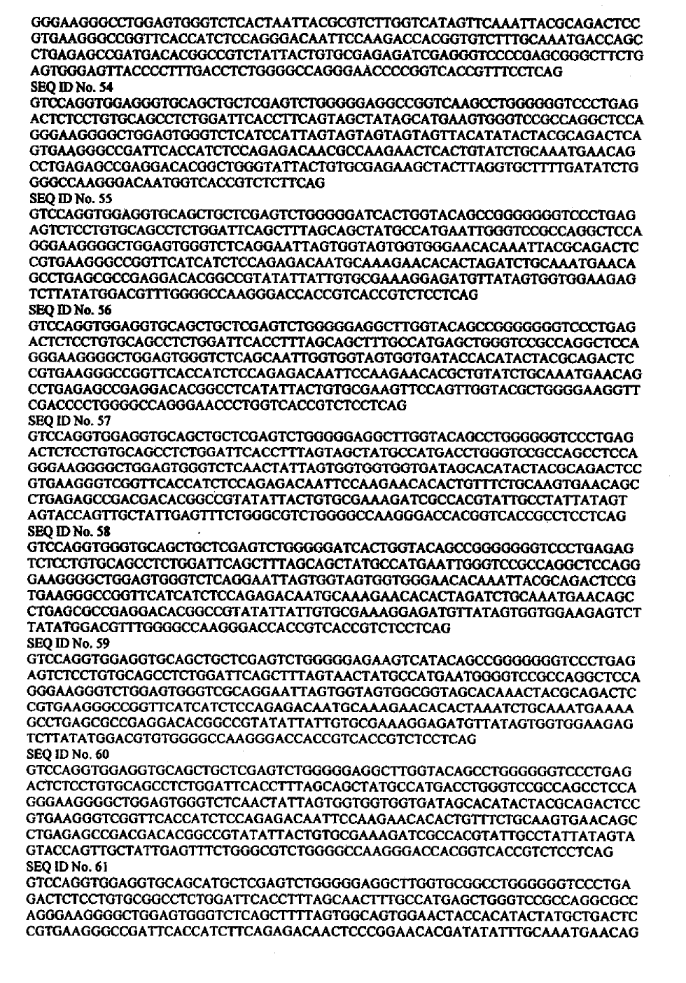

FIG. 1 shows an experimental design. Two groups of Balb/c mice (n=4/group) were sensitized i.p. with rBet v 1/Al(OH)₃ on day 1, 14 and 28. Blood was collected on day 36 (ante-serum). On the same day group 1 was injected i.p. with rBet v 1-specific IgG whereas group 2 obtained IgG for an irrelevant allergen (Phl p 5). Twenty four hours later (day 37) blood was collected (post-serum).

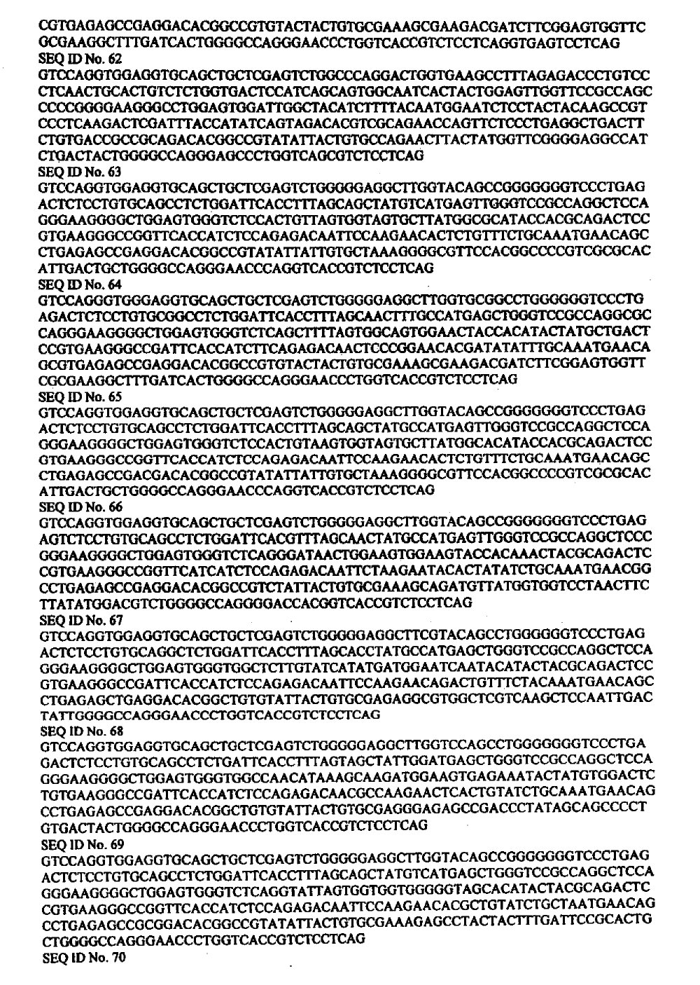

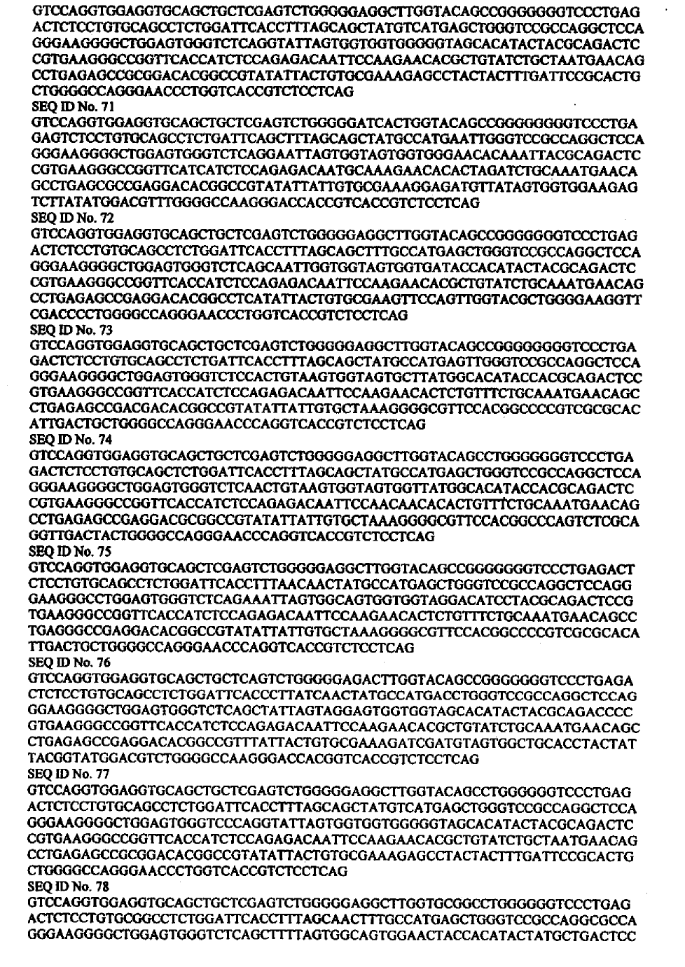

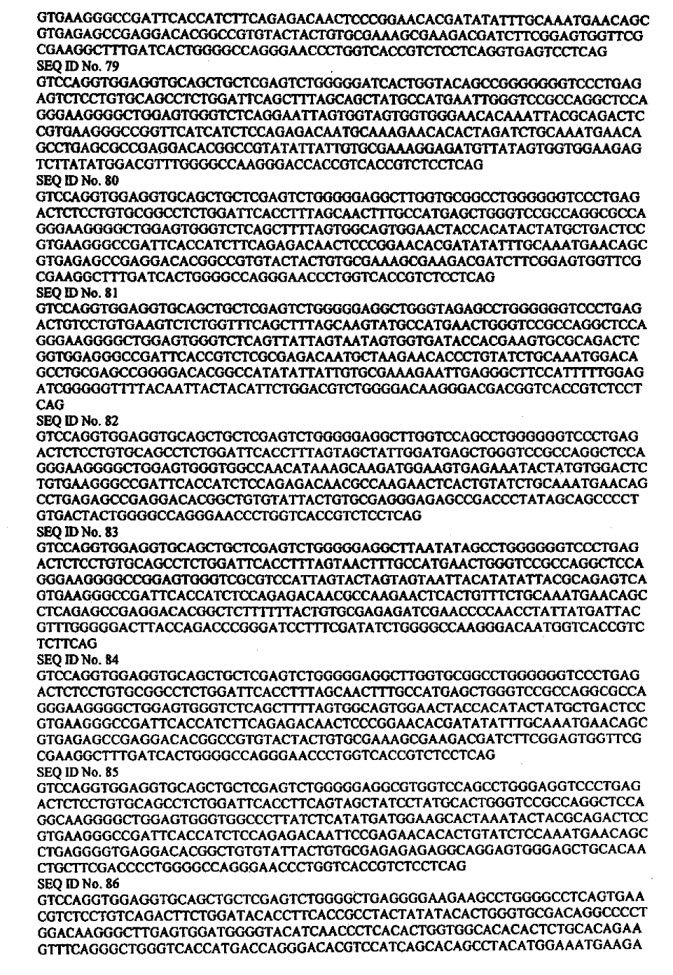

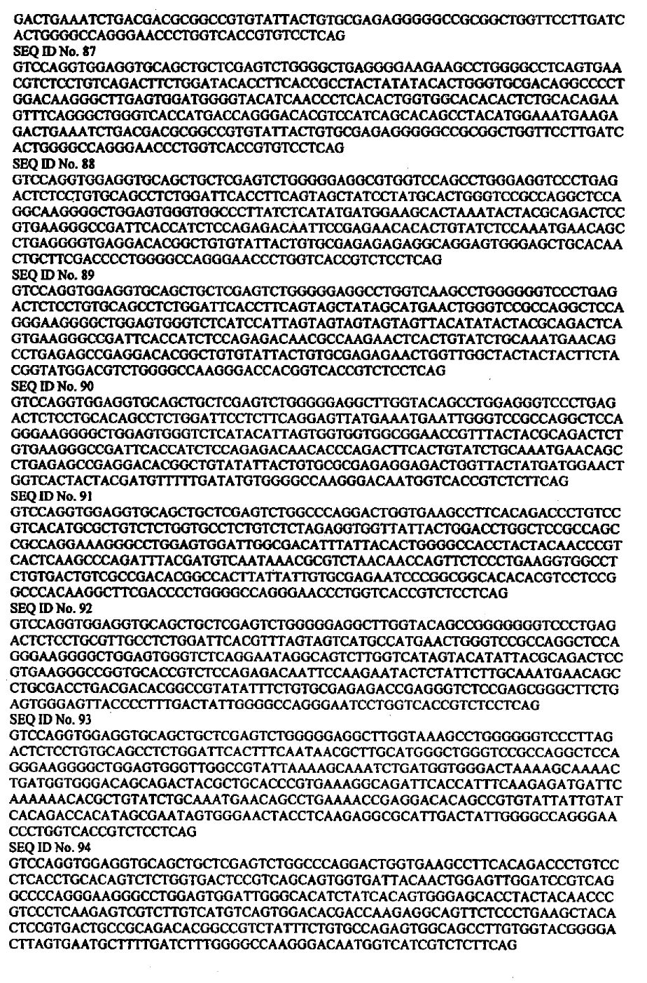

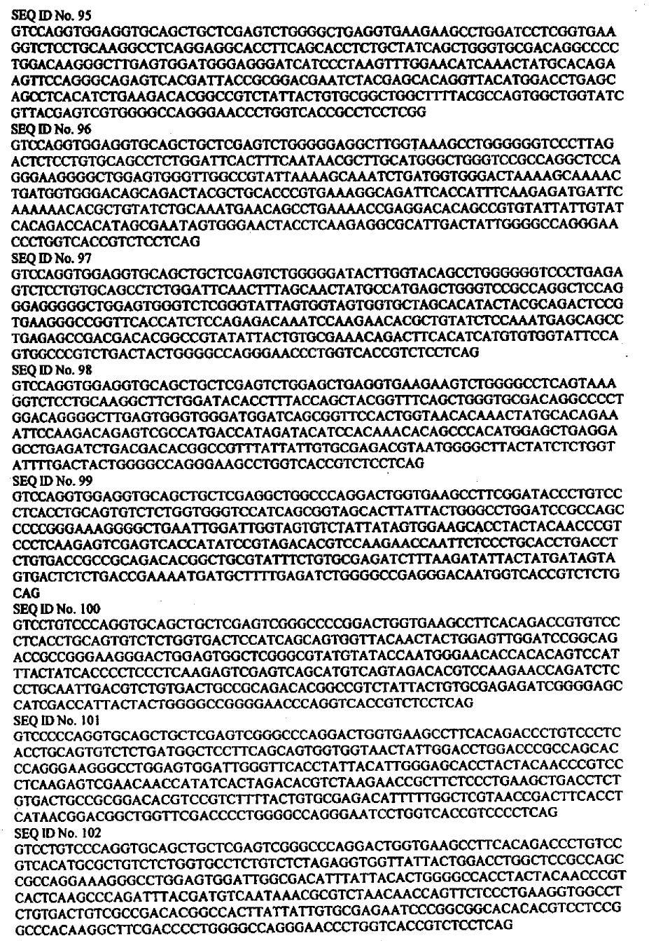

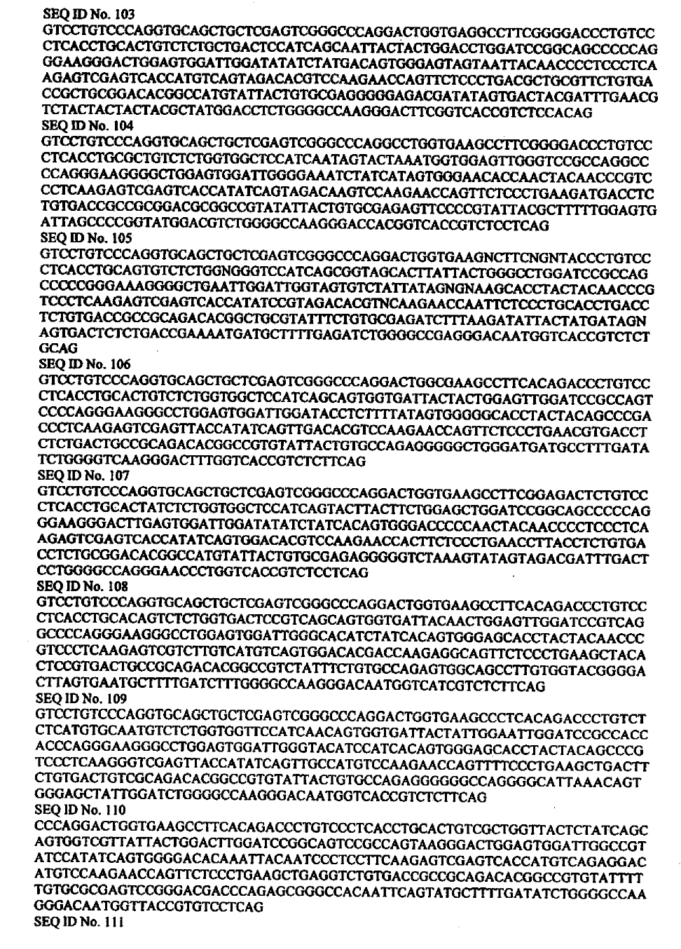

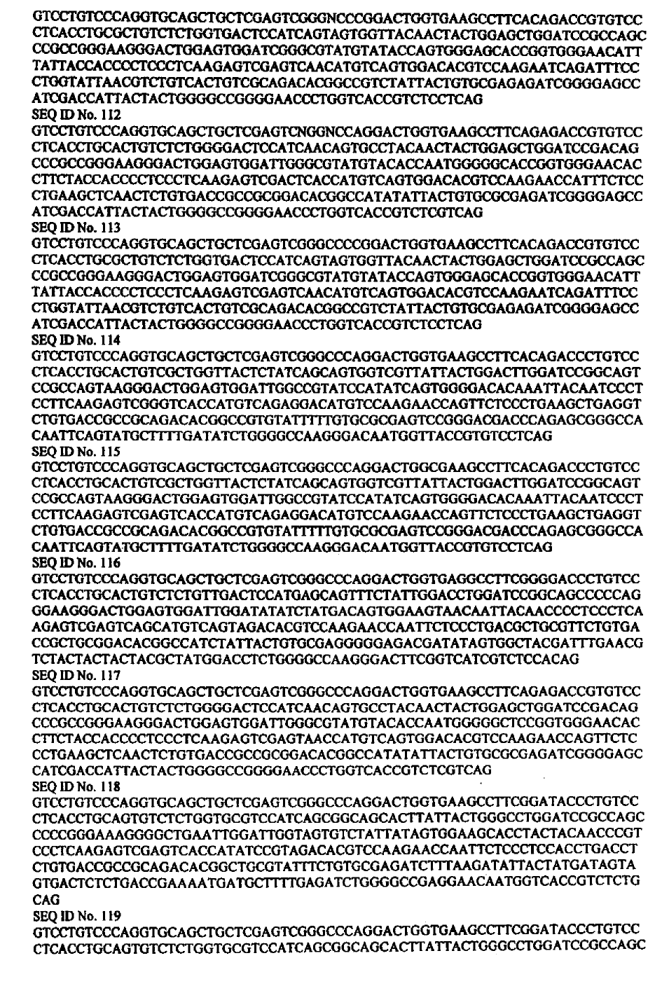

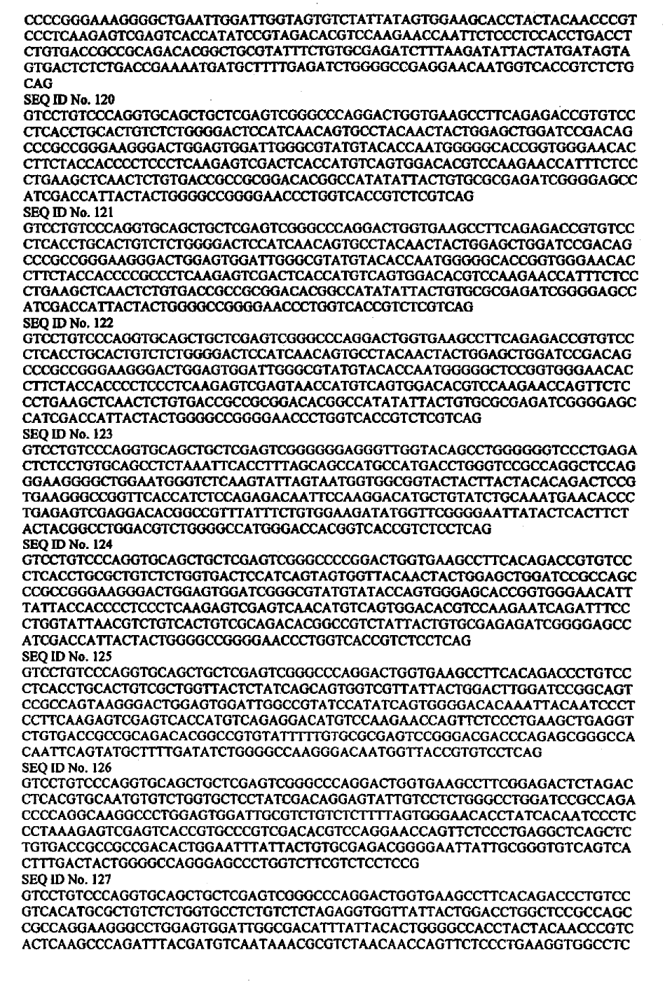

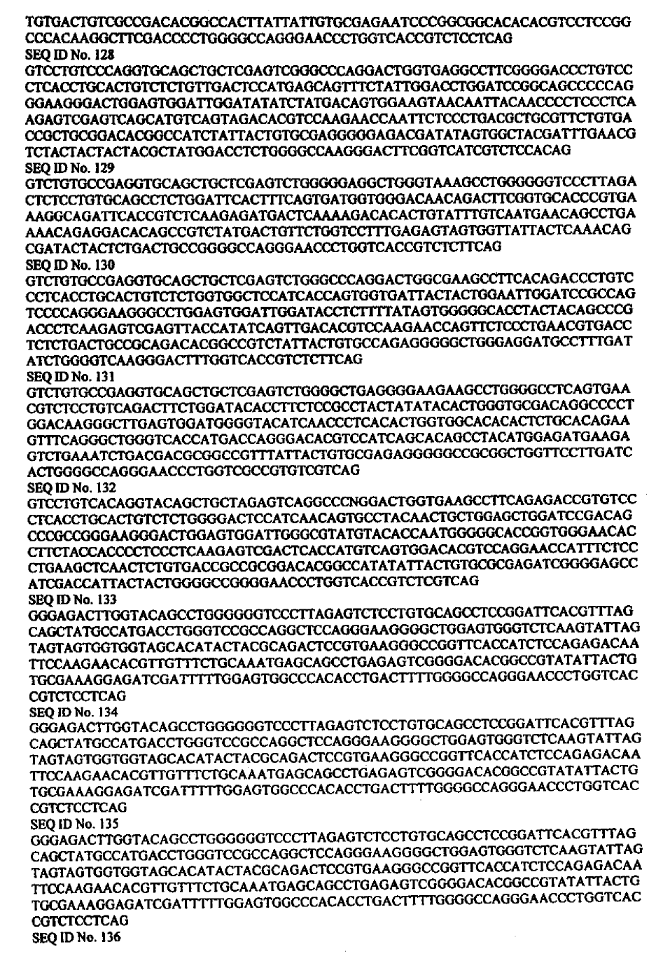

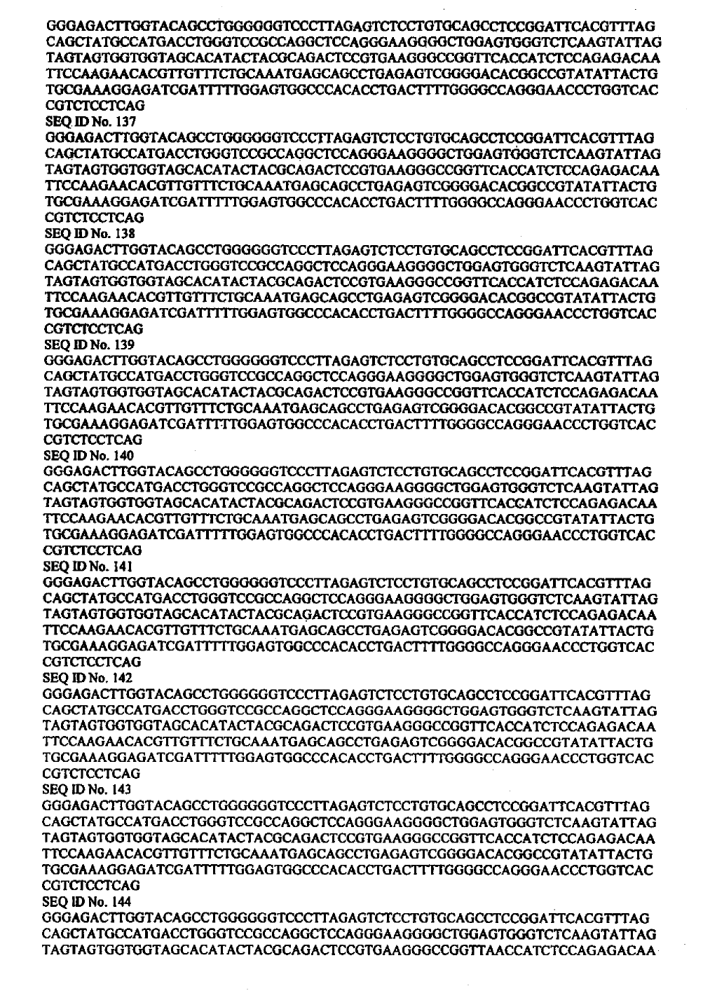

FIG. 2 shows the inhibition of β-hexosaminidase release by rPhl p 5-specific IgG antibodies. Increasing concentrations of rPhl p 5 (0.02 μg/ml-0.5 μg/ml) were preincubated with mouse ante-sera and post-sera, respectively, exposed to RBL cells and the β-hexosaminidase release was measured. The β-hexosaminidase release is expressed as percentage of total β-hexosaminidase release.

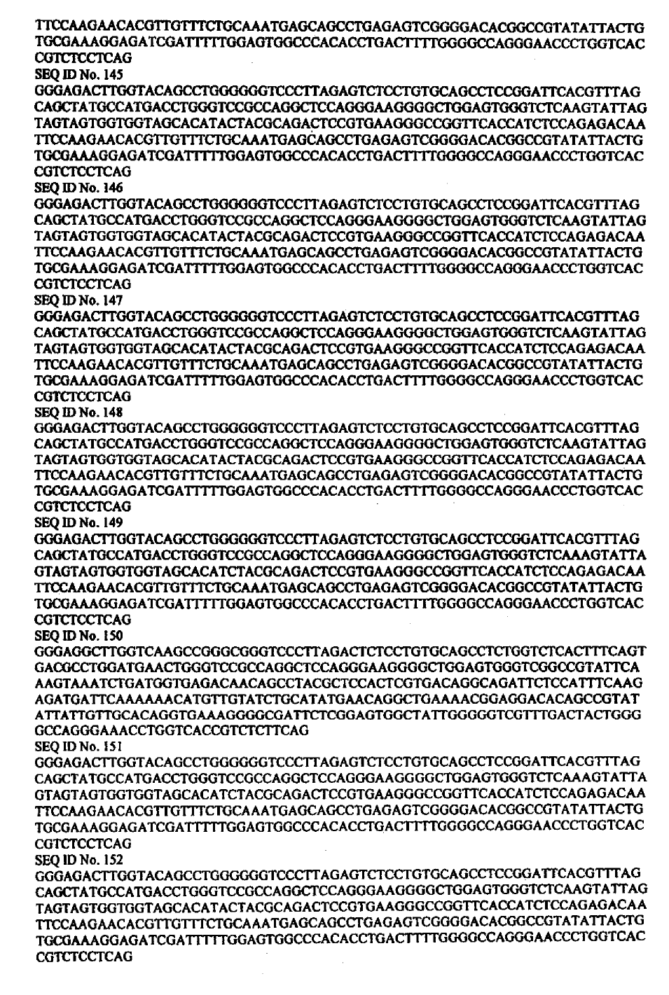

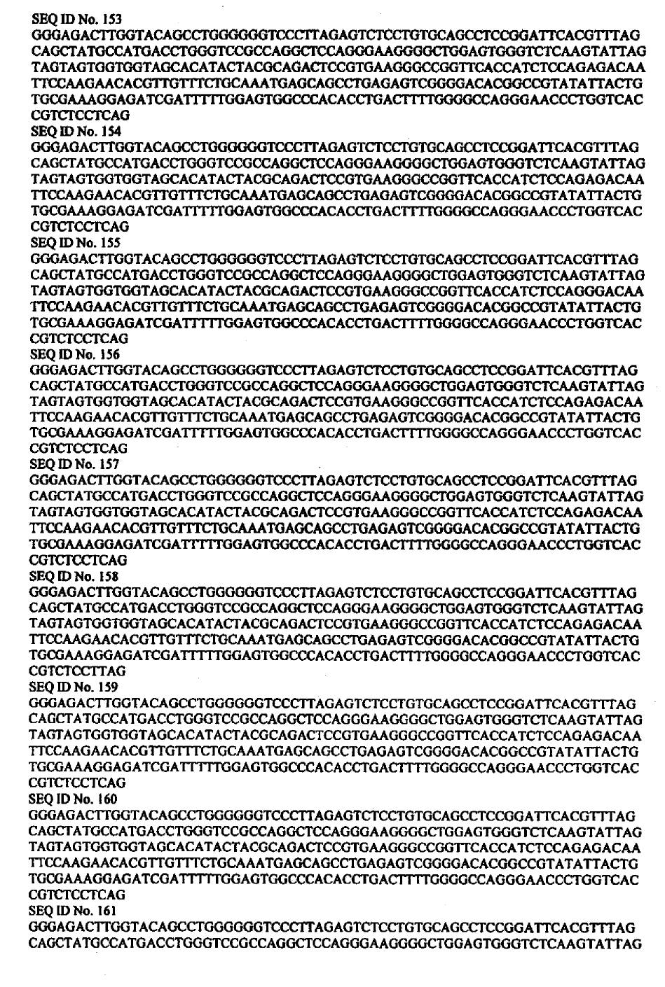

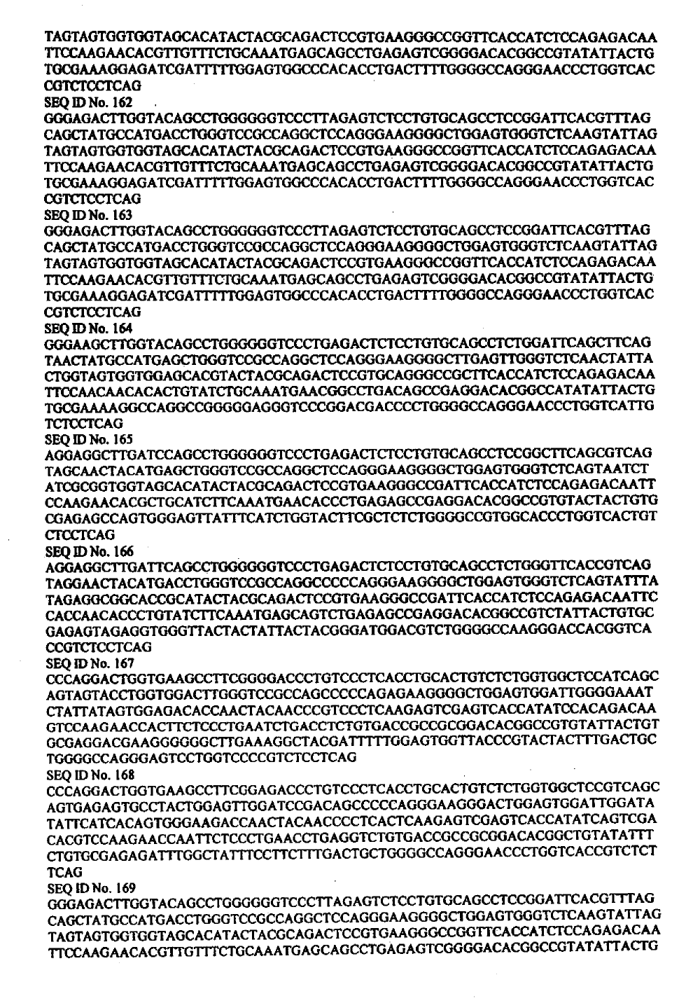

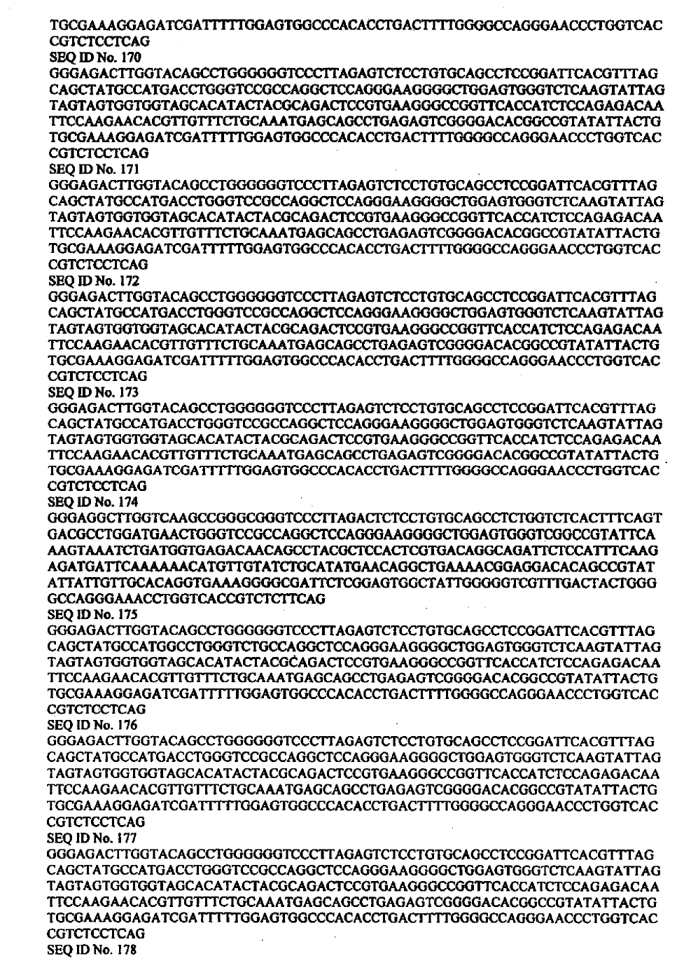

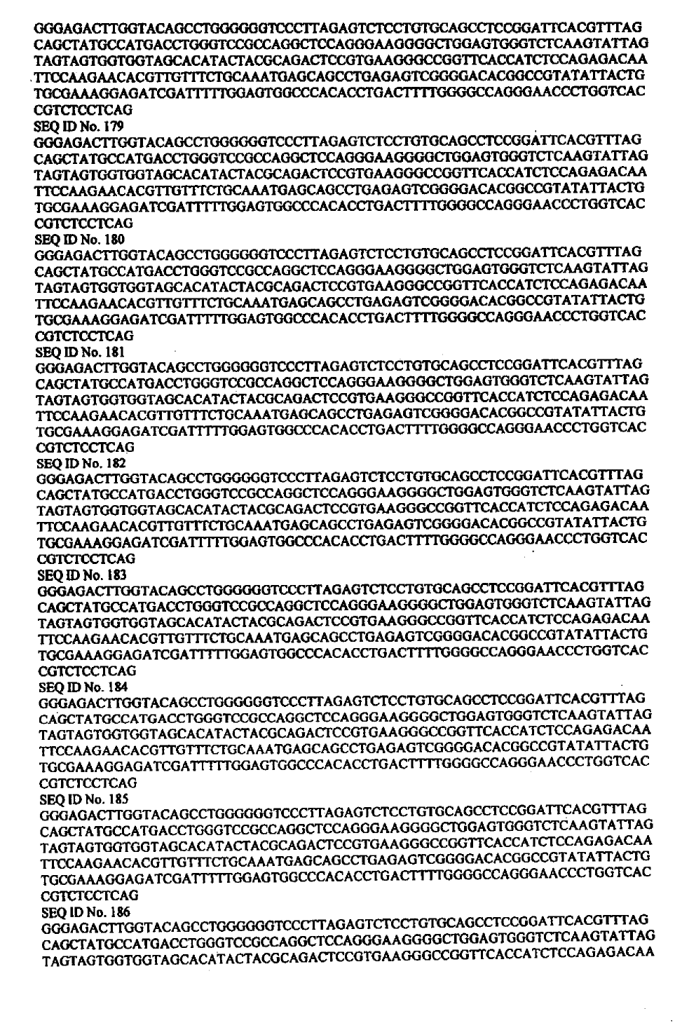

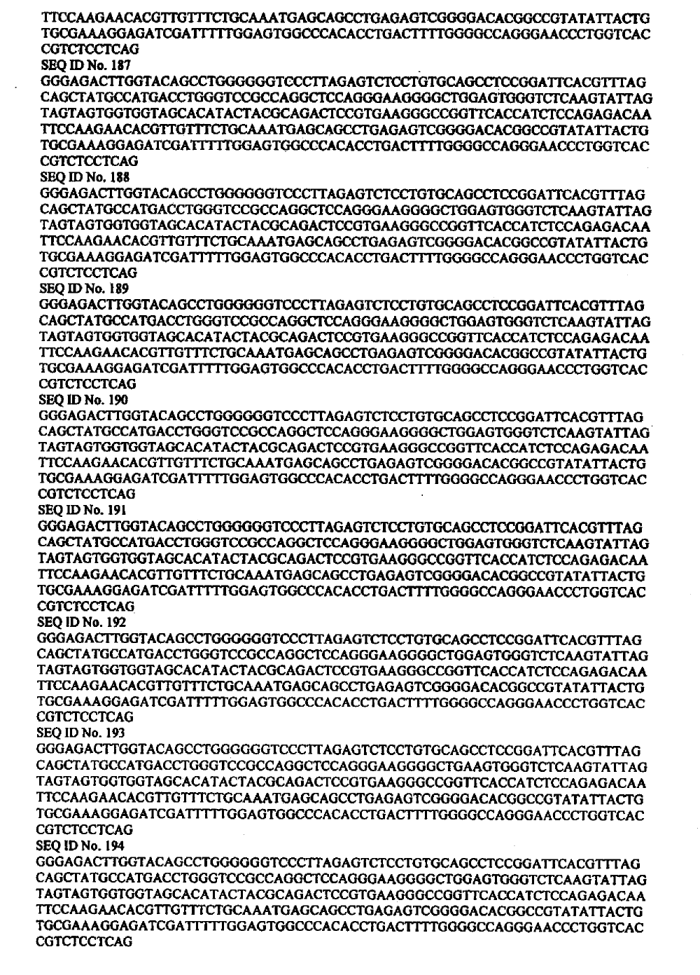

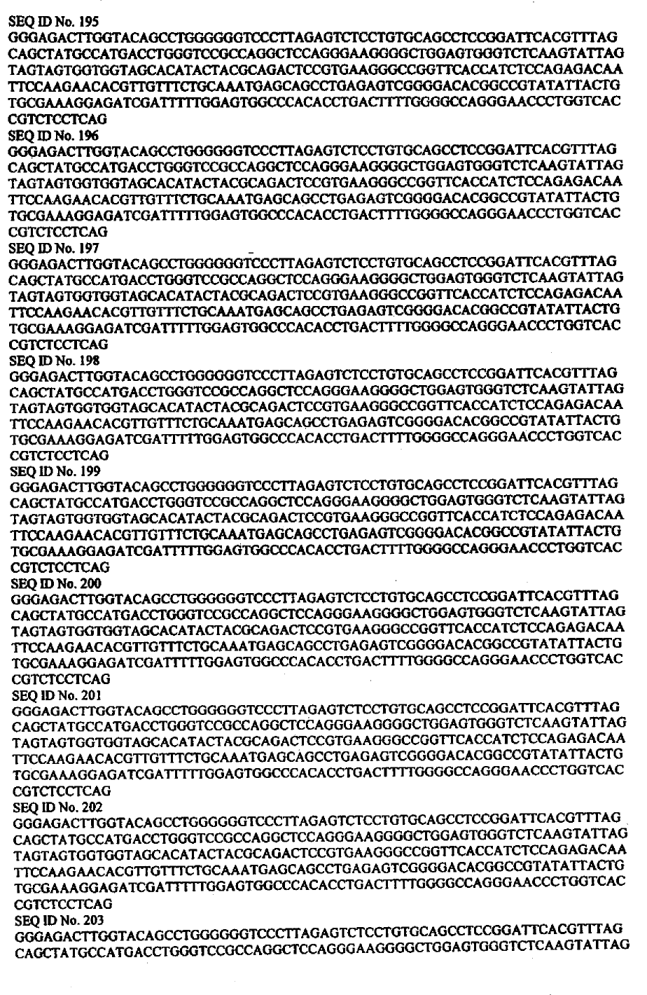

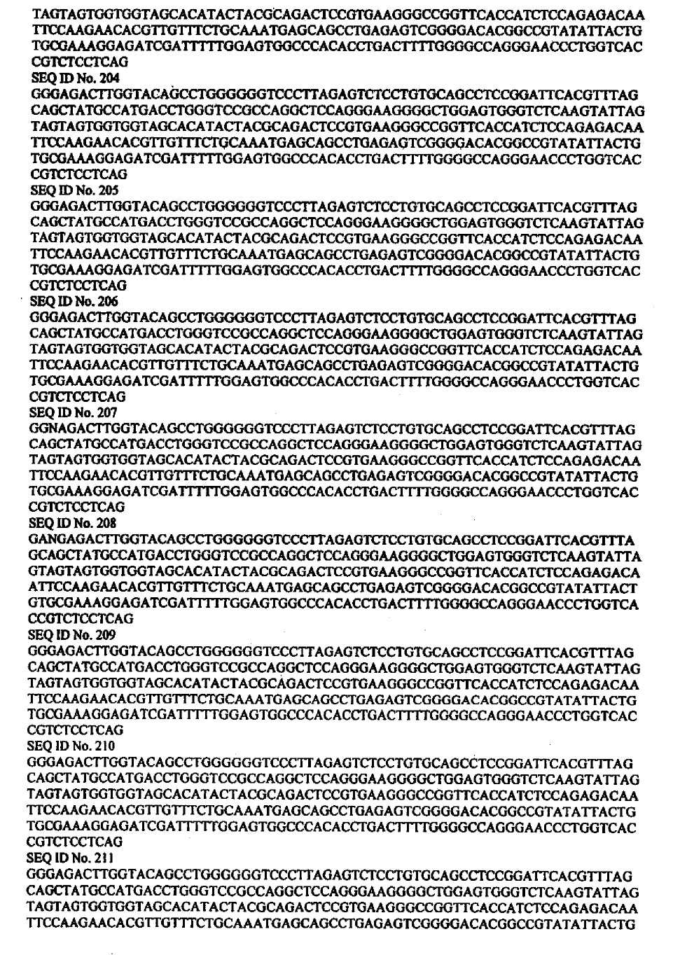

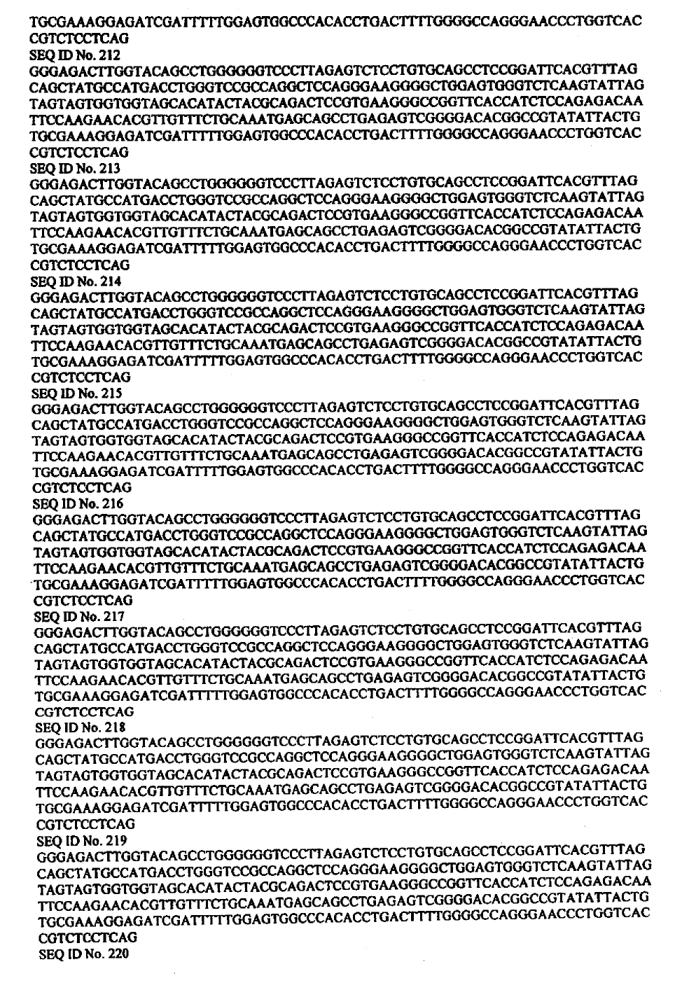

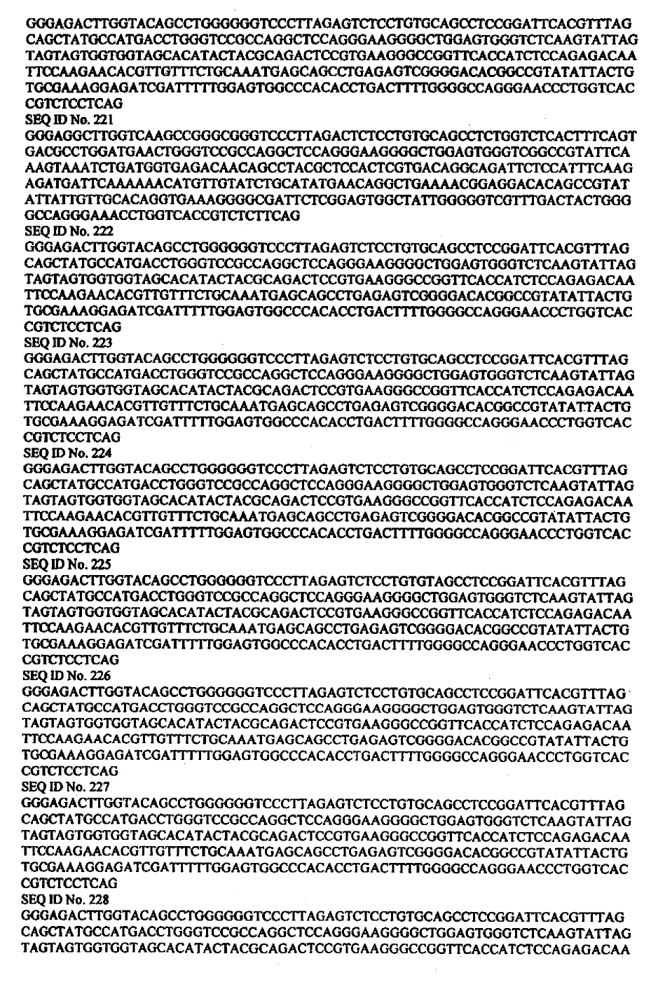

FIG. 3 shows the inhibition of β-hexosaminidase release by rBet v 1-specific IgG antibodies. Increasing concentrations of rBet v 1 (0.02 μg/ml-0.5 μg/ml) were preincubated with mouse ante-sera and post-sera, respectively, exposed to RBL cells and the β-hexosaminidase release was measured. The O-hexosaminidase release is expressed as percentage of total β-hexosaminidase release.

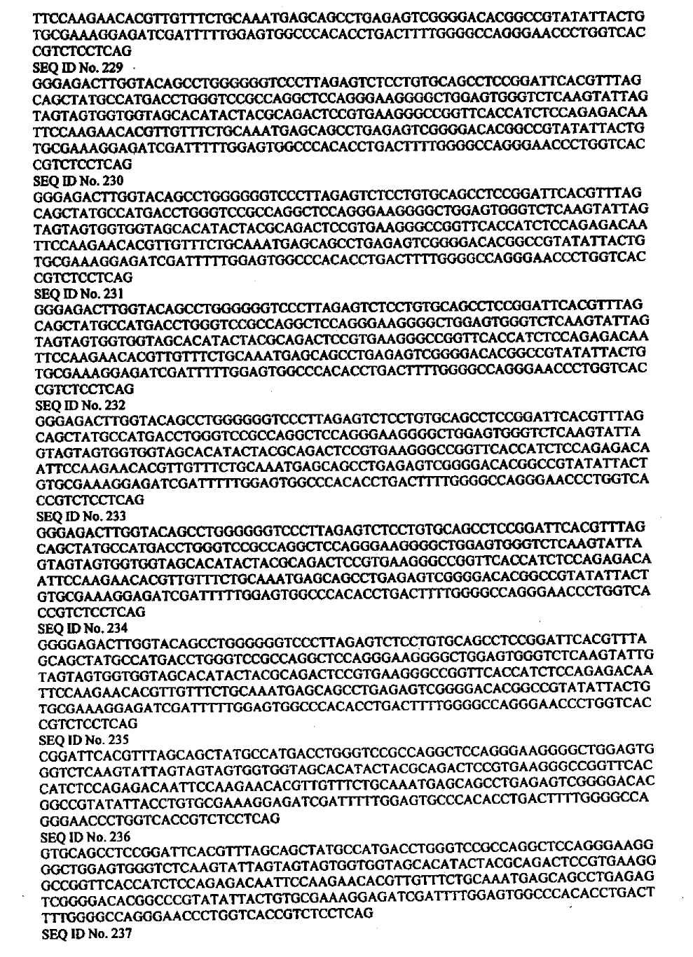

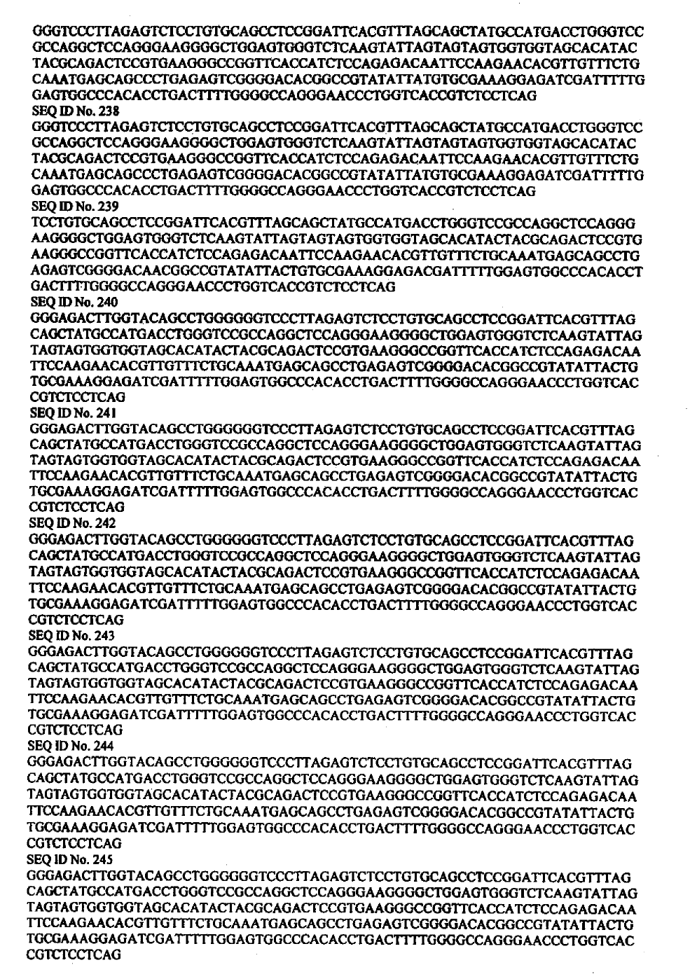

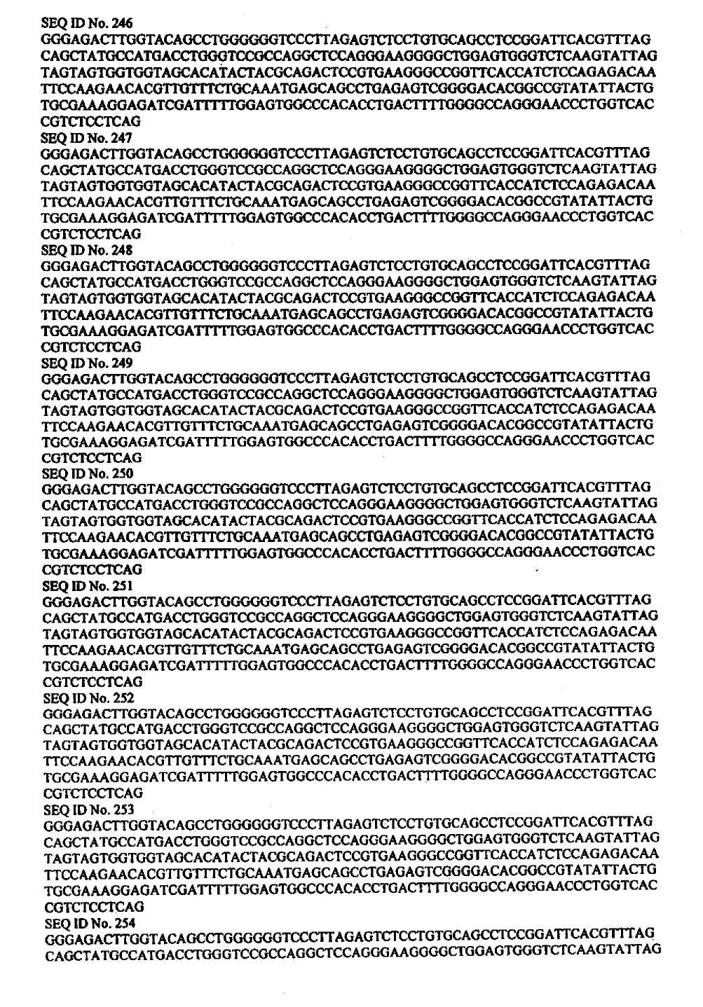

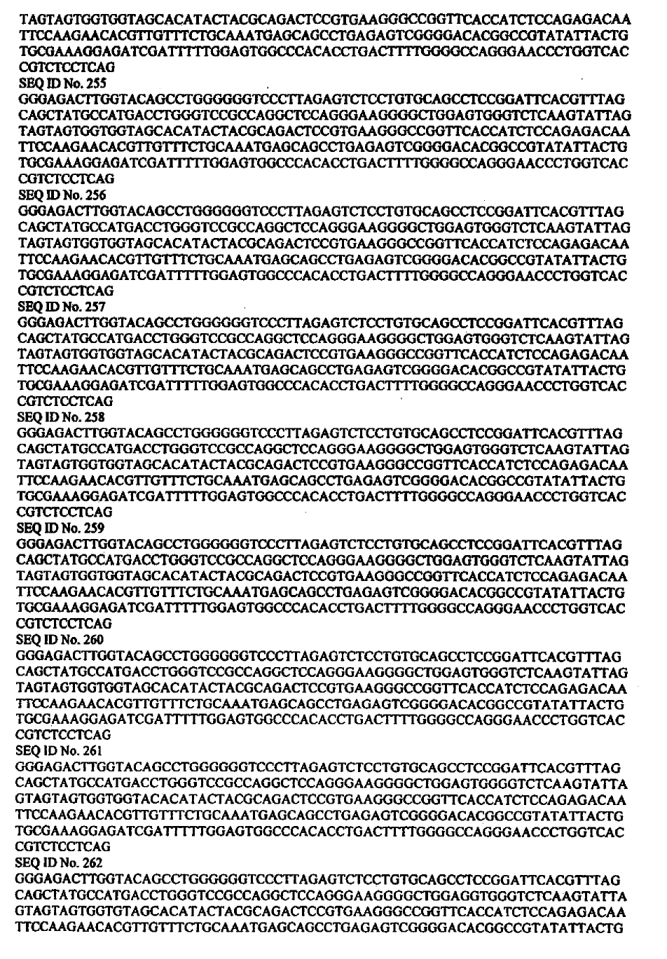

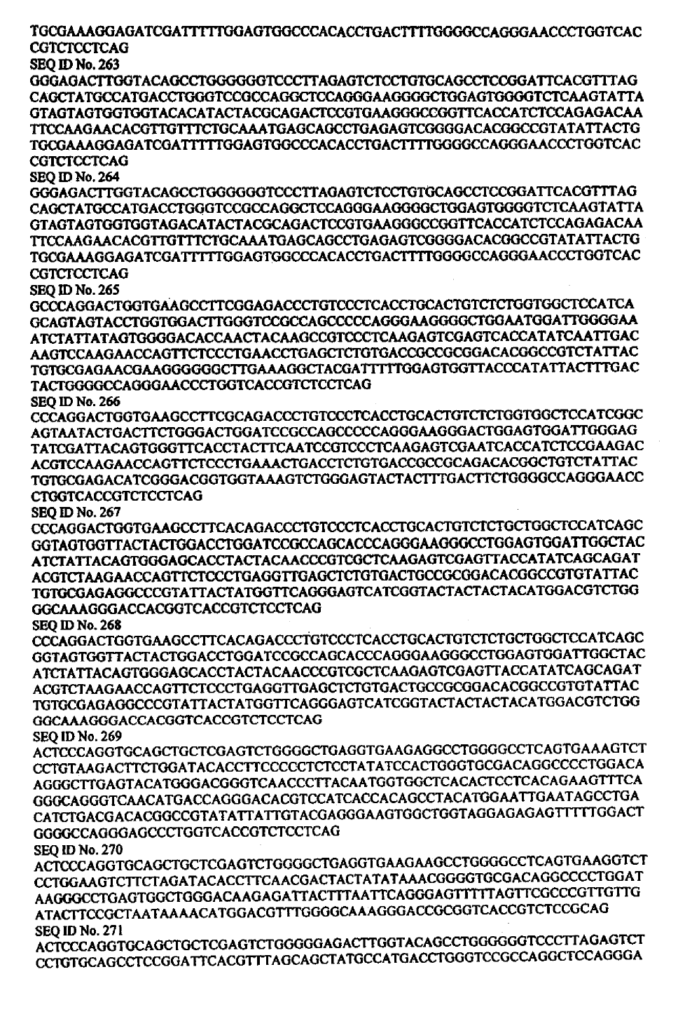

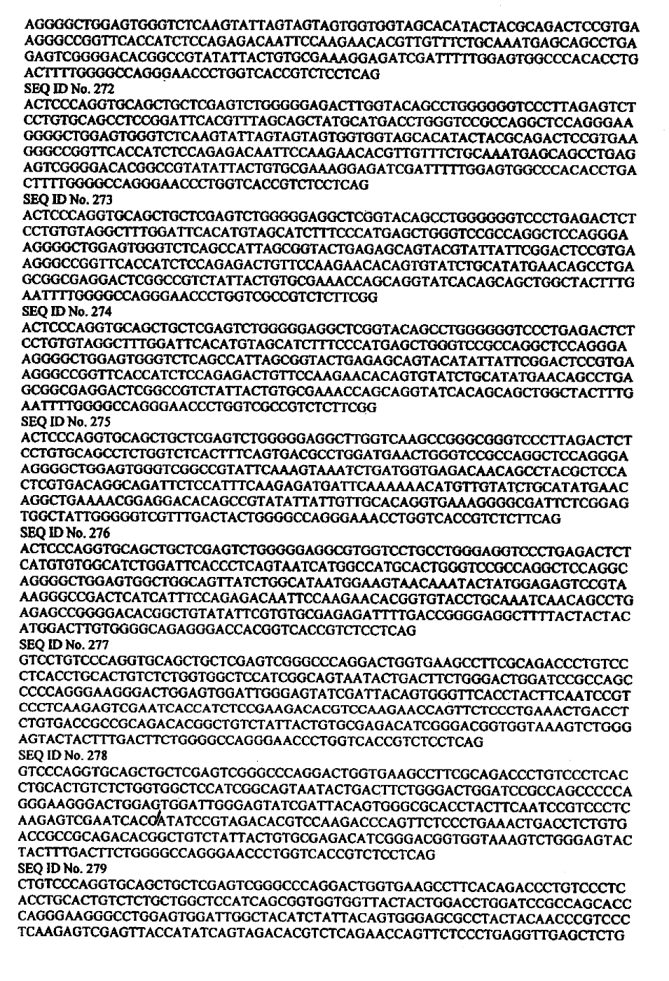

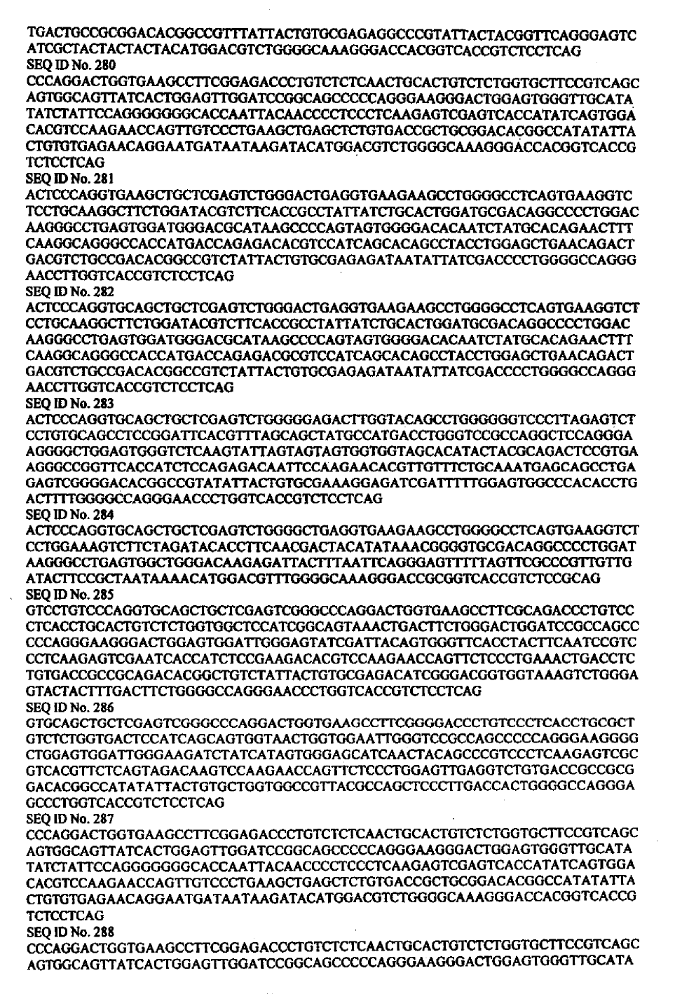

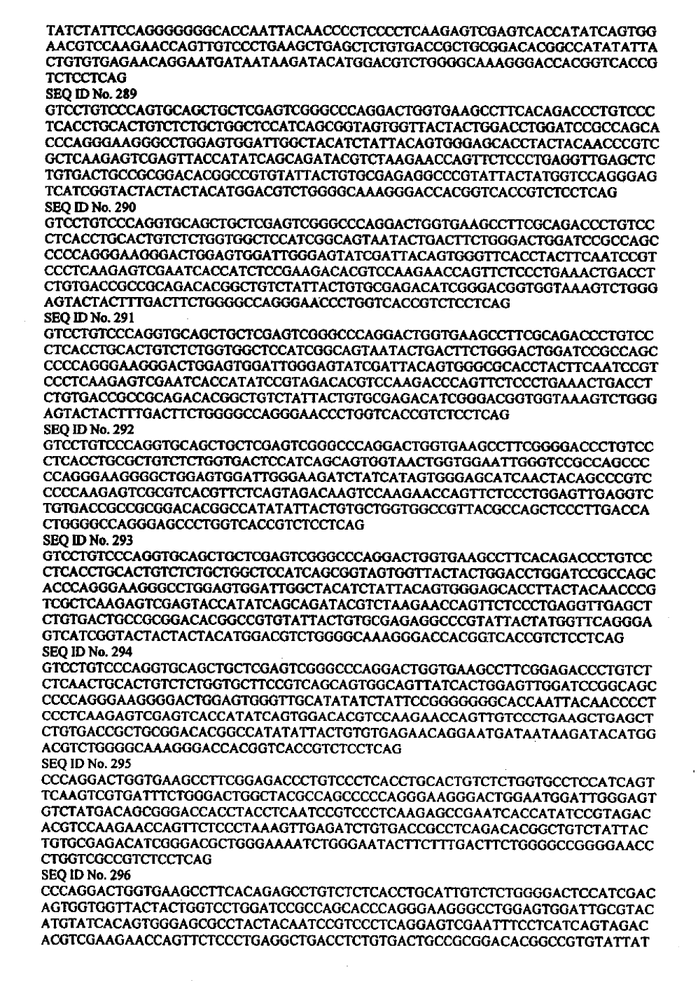

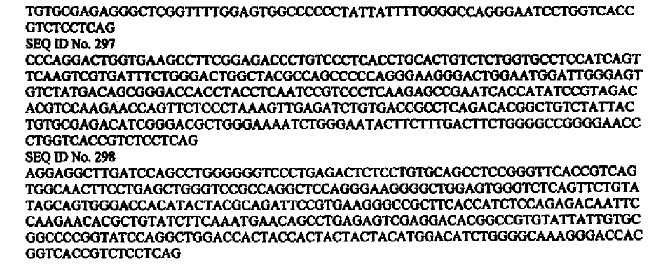

FIG. 4 shows DNA sequences coding for IgE variable regions which are able to bind to Bet v 1.

### EXAMPLES

### Example 1

### Generation and Characterization of Hybridomas Secreting Allergen-Specific Blocking IgG1 Antibodies

### Example 1.1

### Generation of Hybridomas Secreting Allergen Blocking IgG1 Antibodies

Recombinant birch pollen allergen Bet v 1 was expressed in Escherichia coli and purified as described previously (Hoffmann-Sommergruber et al., 1997). Peptides were synthesized on the Applied Biosystems peptide synthesizer Model 433A (Foster City, Calif., USA) and to each of the synthetic peptides one cysteine residue in addition to the original sequence was attached to facilitate coupling to carriers (Focke et al., 2004). Table 1 summarizes the characteristics of the non-anaphylactic Bet v 1-derived synthetic peptides.

TABLE 1
Characteristics of non-anaphylactic Bet v 1-derived peptides

| | | | |
|---|---|---|---|
| | | Number | Molecular |
| Position aa | Sequence | of aa | weight |

| | | | | |
|---|---|---|---|---|
| Peptide 2 | 30-59 | LFPKVAPQAISSVENIEGNGGPGTIKKISFC | 31 | 3202.7 |
| Peptide 6 | 75-104 | CVDHTNFKYNYSVIEGGPIGDTLEKISNEIK | 31 | 3484.9 |

Synthetic peptides (peptide 2 (SEQ ID No. 1), peptide 6 (SEQ ID No. 2)) were coupled to keyhole limpet haemocyanin (KLH: MW 4.5×105 to 1.3×107; Pierce, USA) according to manufacturer's protocol and purified using a Conjugation Kit (Pierce). Balb/c mice (Charles River, Germany) were immunized 3 times (Table 2.) with the KLH-coupled peptide (30 μg/ml per mouse) adsorbed to Al(OH)₃ (75 μl/mouse). The allergen-specific IgG1 titer of sera was determined by ELISA.

TABLE 2
Immunization Schedule

| day | manipulation | adjuvant | site |
|---|---|---|---|
| 0 | Primary immunization | Al(OH)3 | s.c. |
| 28 | Boost #1 | Al(OH)3 | s.c. |
| 46 | Boost #2 | Al(OH)3 | s.c. |
| 49 | Harvest spleen and fuse | Al(OH)3 | s.c. |

Spleen cells were harvested 3 days after the last immunization and the hybridomas were raised by conventional hybridoma technology (Köhler and Milstein, 1975) with slight modifications, using the HAT-sensitive, nonsecreting myeloma cell line X63Ag8.653 (Kearney et al., 1979) as a fusion partner. Myelomas were grown in the hybridoma growth medium consisting of RPMI 1640 supplemented with L-glutamine (200 mM), 10% foetal bovine serum, fungizone (200 U/ml) and penicillin/streptomycin (10000 U/ml). Spleens of mice were removed as mentioned and the cells suspended in serum-free hybridoma growth medium. After centrifugation at 1750 rpm (5 min, 4° C.) the red blood cells were lysed with lysis buffer (8.3 g/l ammonium chloride, 1.0 g/l potassium bicarbonate, 0.037 g/l tetrasodium EDTA, pH 7.4; for 2 min at room temperature) and cells were washed 3 times by centrifugation at 1750 rpm (5 min, 4° C.), each time the cell pellet was resuspended gently with serum-free hybridoma growth medium. Then viable spleen cells and myeloma cells (in log phase of growth) were mixed together in a ratio of 2:1 (spleeh:myeloma) and after centrifugation, 1.5 ml of pre-warmed (37° C.) 41.3% w/v polyethylenglycol (PEG) 4000 was added to the stirred up cell pellet slowly during 1 minute. Then cells were centrifuged at 800 rpm (5 min, 4° C.) and suspended in HAT medium supplemented with feeder cells, distributed in 96-well plates and incubated at 37° C. in a CO₂ incubator (5%). Cells were allowed to grow for around 2 weeks, and afterwards supernatants were screened for antibody production in an enzyme immunoassay.

ELISA plates were coated by overnight incubation at 4° C. with rBet v 1 (10 μg/ml) diluted in PBS. After blocking with 0.5% w/v bovine serum albumin (BSA) in PBS-T (PBS+0.05% Tween 20) for 1 hour at 37° C., plates were incubated with undiluted hybridoma supernatant and were allowed to react for 2 hours at 37° C. For detection, plates were incubated with a 1:1000 diluted primary detection antibody (purified rat anti-mouse IgG1) for 2 hours at 37° C., followed by the 1:2000 diluted secondary enzyme labeled antibody (anti-rat IgG, horseradish peroxidase linked species-specific whole antibody), incubated 30 min each at 37° C. and 4° C. Plates were washed repeatedly with PBS-T between incubation steps. Finally, plates were incubated with ABTS (2,2′-Azino-bis(3-ethylbenzthiazoline-6-sulfonic acid) diammonium salt (Sigma-Aldrich) at room temperature and absorbance was measured at 405 nm. Hybrid cells that secreted IgG1 antibodies specific for Bet v 1 were cloned by the limiting dilution method, i.e., positive hybridomas were expanded, subcloned to assure monoclonality and cryopreserved.

### Example 1.2

### Characterization of Hybridomas Secreting Allergen-Specific Blocking IgG1 Antibodies

For characterization of detailed binding-specificities of the obtained monoclonal IgG1 antibodies by ELISA, microtiter plates were coated with rBet v 1, Peptide 2 (aa 30-59), Peptide 6 (aa 75-104), Bet v 1-Trimer, Bet v 1-Fragment 1 (aa 1-74), Bet v 1-fragment 2 (aa 75-160), KLH and rPhl p 1 at a concentration of 5 μg/ml, diluted in PBS. Blocking was performed by adding 0.5% w/v bovine serum albumin (BSA) in PBS-T (PBS+0.05% Tween 20) for 1.5 hours at 37° C. and thereafter undiluted hybridoma supernatant was incubated for 2 hours at 37° C. Specific binding of mAbs was detected with primary detection antibody followed by secondary enzyme labeled antibody as described above.

Altogether 44 sera from birch-pollen allergic patients (total IgE levels: 24.1->5000 kUA/L; birch pollen-specific IgE: 10.7->100 kUA/L) were selected according to case history, serum from a non-allergic person was included for control purposes. The birch pollen allergic patients group consisted of 19 females and 25 males with a mean age of 37 (ranging from 21 to 70 years). Table 3 summarizes the characteristics of sera from selected birch-pollen allergic patients.

TABLE 3
Characterization of sera from birch-pollen allergic patients

| | | | Total IgE | Birch | RAST |
|---|---|---|---|---|---|
| Patient | Sex | Age | (kUA/l) | (kUA/L) | class |
| a1 | W | 26 | 24.1 | 11.5 | 3 |
| a2 | M | 57 | 172 | 34.6 | 4 |
| a3 | W | 24 | 149 | 20.4 | 4 |
| a4 | W | 50 | 28.1 | 13.2 | 3 |
| a5 | M | 57 | 36.9 | 26.1 | 4 |
| a6 | W | 34 | 84.6 | 16.4 | 3 |
| a7 | W | 35 | 141 | 22 | 4 |
| a8 | W | 30 | 182 | 27.1 | 4 |
| a9 | W | 23 | 278 | 54.5 | 5 |
| a10 | M | 36 | 126 | 11.8 | 3 |
| b1 | M | 26 | 158 | 20.5 | 4 |
| b2 | W | 34 | 41.5 | 21.1 | 4 |
| b3 | W | 38 | 120 | 42.5 | 4 |
| b4 | M | NS | >2000 | >100 | 6 |
| b5 | W | 23 | 423 | 63.3 | 5 |
| b6 | W | 56 | 60.6 | 12.7 | 3 |
| b7 | W | 38 | 273 | 84.6 | 5 |
| b8 | M | 44 | 26.3 | 10.7 | 3 |
| b9 | M | 60 | 235 | 49.6 | 4 |
| b10 | W | 32 | 34.4 | 12.1 | 3 |
| b11 | M | 41 | >2000 | >100 | 6 |
| b12 | M | 26 | 34.7 | 18.6 | 4 |
| b13 | W | 22 | >5000 | >100 | 6 |
| b14 | W | 22 | 560 | >100 | 6 |
| b15 | M | 70 | 112 | 28.4 | 4 |
| b16 | M | 25 | 512 | 94.4 | 5 |
| b17 | M | 44 | 113 | 26.7 | 4 |
| b18 | M | 41 | 94 | 19.8 | 4 |
| b19 | M | 23 | 205 | >100 | 6 |
| b20 | M | NS | 28.3 | 13.3 | 3 |
| b21 | M | NS | NS | 32.1 | 4 |
| b22 | M | 29 | 338 | 16.5 | 3 |
| b23 | M | 57 | >100 | 80.1 | 5 |
| b24 | M | 37 | 95.3 | 41.6 | 4 |
| b25 | M | NS | 252 | 41 | 4 |
| b26 | M | 36 | 50 | 13.7 | 3 |
| b27 | M | 52 | 125 | 51.3 | 5 |
| b28 | M | 23 | 122 | 17.6 | 4 |
| b29 | W | 24 | 49.9 | 27.4 | 4 |
| b30 | M | 53 | 59.7 | 14.1 | 3 |
| b31 | W | 21 | 238 | 21.8 | 4 |
| b32 | W | 21 | 218 | 28.8 | 4 |
| b33 | M | 33 | 72.1 | 11.8 | 3 |
| b34 | W | 31 | 60.1 | 33.1 | 4 |

Individuals are numbered as in Table 6-9. Demographic data show the sex and age of birch pollen allergic patients used for inhibition experiments. Serological characterization displays total IgE, birch pollen-specific IgE and RAST class.
NS, not specified.

ELISA plates were coated with rBet v 1 (1 μg/ml) at 4° C. over night. After blocking with 0.5% w/v bovine serum albumin (BSA) in PBS-T (PBS+0.05% Tween 20) for 1 hour at 37° C., plates were preincubated with undiluted single (clone 2 (DSM ACC2782), 4 (DSM ACC2783), 10 (DSM ACC2785), 12 (DSM ACC2784), 13 (DSM ACC2786)) and mixed (clone 2 and clone 13) IgG1 antibody-producing hybridoma culture supernatant overnight at 4° C. Finally, plates were incubated with 1:5 diluted sera from 44 birch pollen-allergic patients (4° C. o.n.) and bound IgE antibodies were detected with a 1:1000 diluted alkaline-phosphatase-coupled mouse monoclonal anti-human IgE antibody. Plates were washed repeatedly with PBS-T between incubation steps. The percentage inhibition of IgE binding to rBet v 1 after preincubation with IgG1 monoclonal antibody was calculated as follows: % inhibition=100−(ODp×100/ODnp). ODp and ODnp represent the extinctions after preincubation with hypridoma culture supernatant (ODp) and without (ODnp), respectively.

### Example 1.3

### Results

After plating and incubation of fused spleen cells, each supernatants from microtiter wells were analyzed by ELISA as described above, to isolate immunoreactive hybridomas. Further propagation of positive hybridomas resulted in the selection of 14 stable, monoclonal, peptide-specific antibodies. Each one belongs to the IgG1 isotype and expresses the kappa light chain. Table 4. lists the obtained clones. Clones 2, 4, 10, 12 and 13 were deposited under the Budapest Treaty at the Deutsche Sammlung far Mikroorganismen und Zellkulturen (DSM, DSMZ), Braunschweig, Germany, under the deposit numbers DSM ACC2782 (clone 2), DSM ACC27B3 (Clone 4), DSM ACC2785 (clone 10), DSM ACC2784 (clone 12), DSM ACC2786 (clone 13) on 9 May 2006.

TABLE 4
Description of clones

| Clone | Name | Isotype | kappa light chain | Immunogen | DSM Deposit Number |
|---|---|---|---|---|---|
| 1 | P2/3D3/12/2E7 | IgG1 | + | rBet v 1 aa 30-59-KLH | |
| 2 | P2/3D3/10/2D7 | IgG1 | + | rBet v 1 aa 30-59-KLH | ACC2782 |
| 3 | P2/3D3/7/1E6 | IgG1 | + | rBet v 1 aa 30-59-KLH | |
| 4 | P2/6D5/36/2F11 | IgG1 | + | rBet v 1 aa 30-59-KLH | ACC2783 |
| 5 | P2/6D5/34/2P2 | IgG1 | + | rBet v 1 aa 30-59-KLH | |
| 6 | P2/6D5/33/2E7 | IgG1 | + | rBet v 1 aa 30-59-KLH | |
| 7 | P2/6D5/27/1F7 | IgG1 | + | rBet v 1 aa 30-59-KLH | |
| 8 | P2/7G6/58/5G2 | IgG1 | + | rBet v 1 aa 30-59-KLH | |
| 9 | P2/7G6/57/5C3 | IgG1 | + | rBet v 1 aa 30-59-KLH | |
| 10 | P2/7G6/55/4G3 | IgG1 | + | rBet v 1 aa 30-59-KLH | ACC2785 |
| 11 | P2/7G6/54/4F3 | IgG1 | + | rBet v 1 aa 30-59-KLH | |
| 12 | P6/6F6/150/1G7 | IgG1 | + | rBet v 1 aa 75-104-KLH | ACC2784 |
| 13 | P6/7B11/180/2G10 | IgG1 | + | rBet v 1 aa 75-104-KLH | ACC2786 |
| 14 | P6/7B11/178/2G7 | IgG1 | + | rBet v 1 aa 75-104-KLH | |

The 14 peptide-specific antibody producing clones were further tested for their binding properties to rBet v 1, Bet v 1 peptides and Bet v 1 derivatives like the Bet v 1-trimer, consisting of three covalently linked copies of rBet v 1 (Vrtala et al., 2001), and two rBet v 1-fragments, comprising aa 1-74 (1) and aa 75-160 (2) of Bet v 1 (Vrtala et al., 2000). Furthermore, the binding of the monoclonal antibodies to the negative controls, such as KLH and rPhl p 1, was determined. Table 5 summarizes the binding properties of the 14 monoclonal antibodies. None of the 14 peptide-specific antibodies presented any reactivity against KLH or rPhl p 1, however, all of them showed strong reactivity to rBet v 1 and the Bet v 1-trimer. According to the immunogen, clones number 1-11, producing peptide 2-specific (aa 30-59) antibodies, displayed antibody reactivity to this peptide (peptide 2) and failed to react with peptide 6 (aa 72-104). Peptide 6-specific (aa 72-104) antibodies, produced by clones number 12-13, demonstrated binding of the immunogen and lacked binding to peptide 2 (aa 30-59). Further examination, using Bet v 1 fragment 1 (aa 1-74) and fragment 2 (aa 75-106), confirmed that peptide 2-specific antibodies (clones 1-11) showed reactivity to fragment 1 (aa 1-74) whereas peptide 6-specific antibodies (clones 12-13) exhibited reactivity to fragment 2 (aa 75-160).

TABLE 5
Binding properties of IgG1 monoclonal antibodies

| | | rBet v 1 | | | | | | |
|---|---|---|---|---|---|---|---|---|
| | | | fragment 1 | fragment 2 | peptide 2 | peptide 6 | | |
| clone | rBet v 1 | trimer | aa 1-74 | aa 75-160 | aa 30-59 | aa 75-104 | rPhl p 1 | KLH |
| 1 | + | + | + | − | + | − | − | − |
| 2 | + | + | + | − | + | − | − | − |
| 3 | + | + | + | − | + | − | − | − |
| 4 | + | + | + | − | + | − | − | − |
| 5 | + | + | + | − | + | − | − | − |
| 6 | + | + | + | − | + | − | − | − |
| 7 | + | + | + | − | + | − | − | − |
| 8 | + | + | + | − | + | − | − | − |
| 9 | + | + | + | − | + | − | − | − |
| 10 | + | + | + | − | + | − | − | − |
| 11 | + | + | + | − | + | − | − | − |
| 12 | + | + | − | + | − | + | − | − |
| 13 | + | + | − | + | − | + | − | − |
| 14 | + | + | − | + | − | + | − | − |

Table 6 shows that the monoclonal antibodies also inhibit the binding of allergic patients IgE to Bet v 1 cross-reactive allergens such as the major allergen from alder pollen, Aln g 1, or the major allergen from apple, Mal d 1.

TABLE 6
IgG1 monoclonal antibodies inhibit serum IgE binding of birch
pollen-allergic patients to Bet v 1 homologous

| | % Inhibition | | | % Inhibition | | |
|---|---|---|---|---|---|---|
| | Aln g 1 | Aln g 1 | Aln g 1 | Mal d 1 | Mal d 1 | Mal d 1 |
| Patient | clone 2 | clone 4 | clone 10 | clone 2 | clone 4 | clone 10 |
| a1 | 40.95 | 28.25 | 30.79 | 20.56 | 11.85 | 14.63 |
| b3 | 69.13 | 52.22 | 62.37 | 30.67 | 5.14 | 7.51 |
| b16 | 52.82 | 28.32 | 44.70 | 41.22 | 19.35 | 28.32 |
| b9 | 74.63 | 48.34 | 63.88 | 35.16 | 14.37 | 18.06 |
| b15 | 45.14 | 29.81 | 39.05 | 29.91 | 15.33 | 24.30 |
| b14 | 47.92 | 20.37 | 41.42 | 35.58 | 22.79 | 17.22 |
| b20 | 61.73 | 39.42 | 54.23 | 31.45 | 20.16 | 22.96 |
| b19 | 44.72 | 25.04 | 35.59 | 32.29 | 20.38 | 18.18 |
| b26 | 9.40 | 6.04 | 11.41 | 9.32 | 6.21 | 8.07 |
| b33 | 1.06 | 2.13 | 0.00 | 41.22 | 24.90 | 29.80 |
| Mean | 44.8 | 28.0 | 38.3 | 30.8 | 16.0 | 18.9 |

The Percentage inhibitions of IgE binding to complete rBet v 1 obtained with the IgG1 mAbs (clone 2, 4, 10) are displayed for sera from 10 birch pollen allergic patiens.
The mean percentage inhibitions are shown at the bottom of the table.

The capacity of peptide-specific antibodies to inhibit the binding of allergic patients IgE to complete rBet v 1 was determined by ELISA competition experiments (Table 7-9), using sera from 44 birch pollen-allergic patients. Five out of a total of 14 clones were chosen for preincubation with rBet v 1 prior to patients IgE exposure. The strongest inhibition of IgE binding was observed after preincubation with peptide 6-specific (aa 75-104) antibodies (clone 12: 60.4%-74.8% average inhibition; clone 13: 58.5%-72.6% average inhibition), whereas peptide 2-specific (aa 30-59) antibodies (clone 2: 46.2%-62.7% average inhibition; clone 4: 44.3% and 58.7% average inhibition and clone 10: 41.0% and 52.4% average inhibition), also inhibited patients IgE binding to rBet v 1, albeit to a lower extent. Interestingly, a mixture of a peptide 2-specific monoclonal antibody (clone 2) and a peptide 6-specific monoclonal antibody did not exhibit a stronger inhibition of IgE binding than the single monoclonal antibodies alone (Table 7-9).

TABLE 7
IgG1 monoclonal antibodies inhibit serum IgE binding of birch pollen-allergic patients to rBet v 1

| | | OD with mAb | | | | | % Inhibition | | | | |
|---|---|---|---|---|---|---|---|---|---|---|---|
| Patient | OD without mAb | clone 2 | clone 4 | clone 10 | clone 12 | clone 13 | clone 2 | clone 4 | clone 10 | clone 12 | clone 13 |
| a1 | 0.45 | 0.34 | 0.34 | 0.37 | 0.36 | 0.36 | 23 | 25 | 17 | 20 | 19 |
| a2 | 0.74 | 0.34 | 0.36 | 0.40 | 0.21 | 0.20 | 54 | 51 | 46 | 72 | 73 |
| a3 | 0.93 | 0.49 | 0.52 | 0.58 | 0.61 | 0.66 | 47 | 45 | 38 | 34 | 29 |
| a4 | 0.61 | 0.23 | 0.23 | 0.26 | 0.15 | 0.13 | 62 | 62 | 57 | 76 | 78 |
| a5 | 1.19 | 0.81 | 0.87 | 0.86 | 0.43 | 0.58 | 32 | 27 | 28 | 64 | 52 |
| a6 | 0.41 | 0.20 | 0.22 | 0.24 | 0.12 | 0.13 | 51 | 47 | 42 | 71 | 69 |
| a7 | 0.62 | 0.33 | 0.35 | 0.38 | 0.19 | 0.22 | 48 | 44 | 38 | 70 | 65 |
| a8 | 0.63 | 0.31 | 0.32 | 0.33 | 0.17 | 0.20 | 50 | 49 | 47 | 73 | 69 |
| a9 | 0.42 | 0.19 | 0.19 | 0.19 | 0.10 | 0.09 | 55 | 54 | 54 | 76 | 78 |
| a10 | 0.38 | 0.23 | 0.23 | 0.22 | 0.19 | 0.18 | 40 | 39 | 43 | 49 | 54 |
| control | 0.06 | 0.09 | 0.09 | 0.09 | 0.14 | 0.15 | 0 | 0 | 0 | 0 | 0 |
| Mean | | | | | | | 46.23 | 44.25 | 40.97 | 60.42 | 58.53 |

The percentage inhibitions of IgE binding to complete rBet v 1 obtained with the IgG1 mAbs (clone 2, 4, 10, 12, 13) are displayed for sera from 10 birch pollen allergic patiens (a1-a10), serum from, a non-allergic person serves as control.
The mean percentage inhibitions are shown at the bottom at the table.

TABLE 8
IgG1 monoclonal antibodies inhibit serum IgE binding of birch pollen-allergic patients to rBet v 1

| | | OD with mAb | | | | | % Inhibition | | | | |
|---|---|---|---|---|---|---|---|---|---|---|---|
| Patient | OD without mAb | clone 2 | clone 4 | clone 10 | clone 12 | clone 13 | clone 2 | clone 4 | clone 10 | clone 12 | clone 13 |
| b1 | 0.35 | 0.09 | 0.10 | 0.13 | 0.04 | 0.06 | 75 | 72 | 63 | 84 | 84 |
| b2 | 0.29 | 0.09 | 0.11 | 0.13 | 0.07 | 0.07 | 70 | 64 | 56 | 77 | 75 |
| b3 | 0.52 | 0.13 | 0.18 | 0.23 | 0.06 | 0.12 | 74 | 66 | 55 | 85 | 77 |
| b4 | 1.06 | 0.25 | 0.24 | 0.34 | 0.10 | 0.12 | 76 | 77 | 66 | 90 | 89 |
| b5 | 0.70 | 0.26 | 0.31 | 0.37 | 0.09 | 0.12 | 62 | 55 | 46 | 87 | 82 |
| b6 | 0.12 | 0.04 | 0.06 | 0.06 | 0.04 | 0.04 | 70 | 51 | 45 | 70 | 67 |
| b7 | 0.73 | 0.29 | 0.29 | 0.38 | 0.13 | 0.18 | 61 | 60 | 47 | 82 | 75 |
| b8 | 0.13 | 0.06 | 0.06 | 0.07 | 0.05 | 0.05 | 57 | 56 | 49 | 63 | 65 |
| b9 | 0.33 | 0.09 | 0.10 | 0.13 | 0.06 | 0.04 | 73 | 71 | 61 | 83 | 82 |
| b10 | 0.11 | 0.06 | 0.07 | 0.07 | 0.06 | 0.06 | 47 | 39 | 37 | 47 | 47 |
| b11 | 1.24 | 0.28 | 0.30 | 0.40 | 0.08 | 0.12 | 78 | 76 | 68 | 94 | 91 |
| b12 | 0.17 | 0.04 | 0.06 | 0.06 | 0.03 | 0.04 | 75 | 66 | 63 | 79 | 78 |
| b13 | 0.88 | 0.16 | 0.16 | 0.22 | 0.08 | 0.08 | 82 | 81 | 75 | 91 | 90 |
| b14 | 0.61 | 0.76 | 0.77 | 0.95 | 0.29 | 0.46 | 53 | 52 | 41 | 82 | 72 |
| b15 | 0.30 | 0.15 | 0.16 | 0.18 | 0.12 | 0.12 | 51 | 48 | 40 | 61 | 60 |
| b16 | 1.52 | 0.80 | 0.85 | 0.89 | 0.35 | 0.56 | 48 | 44 | 42 | 77 | 63 |
| b17 | 0.25 | 0.06 | 0.09 | 0.11 | 0.04 | 0.06 | 67 | 63 | 56 | 82 | 75 |
| b18 | 0.16 | 0.09 | 0.09 | 0.11 | 0.07 | 0.08 | 45 | 43 | 34 | 59 | 54 |
| b19 | 0.51 | 0.13 | 0.14 | 0.17 | 0.06 | 0.06 | 75 | 72 | 66 | 89 | 88 |
| b20 | 0.11 | 0.07 | 0.08 | 0.08 | 0.06 | 0.06 | 33 | 30 | 29 | 42 | 42 |
| b21 | 0.54 | 0.17 | 0.21 | 0.24 | 0.06 | 0.09 | 68 | 62 | 54 | 89 | 83 |
| b22 | 0.21 | 0.12 | 0.12 | 0.12 | 0.09 | 0.09 | 42 | 44 | 40 | 56 | 57 |
| b23 | 0.52 | 0.13 | 0.14 | 0.17 | 0.06 | 0.06 | 75 | 73 | 67 | 89 | 89 |
| b24 | 0.30 | 0.08 | 0.09 | 0.10 | 0.04 | 0.04 | 73 | 70 | 65 | 86 | 86 |
| b25 | 0.40 | 0.15 | 0.15 | 0.18 | 0.10 | 0.11 | 63 | 61 | 55 | 74 | 73 |
| b26 | 0.16 | 0.09 | 0.09 | 0.09 | 0.08 | 0.07 | 46 | 44 | 42 | 50 | 55 |
| b27 | 0.42 | 0.14 | 0.13 | 0.17 | 0.07 | 0.07 | 68 | 65 | 59 | 84 | 84 |
| b28 | 0.16 | 0.06 | 0.07 | 0.08 | 0.05 | 0.05 | 61 | 57 | 53 | 70 | 71 |
| b29 | 0.31 | 0.13 | 0.14 | 0.17 | 0.07 | 0.09 | 59 | 55 | 47 | 76 | 71 |
| b30 | 0.12 | 0.05 | 0.07 | 0.07 | 0.05 | 0.06 | 53 | 38 | 37 | 55 | 52 |
| b31 | 0.35 | 0.15 | 0.16 | 0.18 | 0.11 | 0.12 | 55 | 53 | 49 | 68 | 66 |
| b32 | 0.25 | 0.07 | 0.08 | 0.09 | 0.06 | 0.06 | 73 | 70 | 65 | 76 | 79 |
| b33 | 0.11 | 0.05 | 0.05 | 0.06 | 0.04 | 0.04 | 55 | 52 | 48 | 65 | 65 |
| b34 | 0.39 | 0.12 | 0.13 | 0.16 | 0.06 | 0.06 | 69 | 47 | 60 | 84 | 85 |
| control | 0.03 | 0.03 | 0.04 | 0.03 | 0.04 | 0.08 | 4 | 0 | 0 | 0 | 0 |
| Mean | | | | | | | 62.69 | 58.68 | 52.37 | 74.84 | 72.64 |

The percentage inhibitions of IgE binding to complete rBet v 1 obtained with the IgG1 mAbs (clone 2, 4, 10, 12, 13) are displayed for sera from 34 birch pollen allergic patiens (b1-b34), serum from, a non-allergic person serves as control.
The mean percentage inhibitions are shown at the bottom of the table.

TABLE 9
Inhibition of serum IgE binding of birch pollen-allergic patients to rBet v 1.
Comparison of the inhibition potency of two single monoclonal antibodies and a mixture of these

| | | OD with mAb | | | % Inhibition | | |
|---|---|---|---|---|---|---|---|
| Patient | OD without mAb | clone 2 | clone 13 | clone 2/13 mix | clone 2 | clone 13 | clone 2/13 mix |
| a1 | 0.30 | 0.13 | 0.07 | 0.07 | 56 | 75 | 75 |
| a2 | 0.29 | 0.11 | 0.07 | 0.07 | 61 | 76 | 74 |
| a3 | 0.19 | 0.07 | 0.05 | 0.04 | 63 | 76 | 78 |
| a4 | 0.17 | 0.08 | 0.05 | 0.05 | 56 | 72 | 70 |
| a5 | 0.03 | 0.03 | 0.03 | 0.05 | 15 | 6 | 0 |
| a6 | 0.14 | 0.09 | 0.07 | 0.09 | 36 | 48 | 37 |
| a7 | 0.25 | 0.09 | 0.06 | 0.06 | 64 | 75 | 75 |
| a8 | 0.24 | 0.10 | 0.08 | 0.07 | 60 | 68 | 72 |
| a9 | 0.18 | 0.06 | 0.04 | 0.04 | 65 | 76 | 77 |
| a10 | 0.30 | 0.16 | 0.11 | 0.09 | 47 | 64 | 70 |
| Mean | | | | | 52.36 | 63.56 | 62.88 |

The percentage inhibitions of IgE binding to complete rBet v 1 obtained with the single IgG1 mAbs (clone 2, clone 13) compared with the inhibition by a mixture of IgG1 mAbs (clone 2/13 mix). Inhibitions are displayed for sera from 10 birch pollen allergic patiens (a1-a10).
The mean percentage inhibitions are shown at the bottom of the table.

The key event of the allergic reaction is the cross-linking of effector-cell bound IgE antibodies by multivalent allergens. This leads to granule exocytosis and biological mediator release (i.e., histamine, leukotrienes), which then causes immediate type allergic inflammation and thus allergic rhinitis, conjunctivitis and asthma. In this respect, the allergen-IgE antibody interaction is a possible target for allergen-specific passive immunotherapy with the aim to inhibit the interaction between allergens and IgE antibodies (Valenta et al., 1998). For this reason the definition of IgE epitopes is an important prerequisite for the development of specific forms of therapy. In the case of major allergens, such as Phl p 1, with continuous IgE epitopes, it is possible to dissect allergens into IgE-binding haptens, which saturate effector-cell bound IgE prior to allergen exposure and thereby prevent cross-linking and effector-cell activation (Ball et al., 1994) (see also chapter 1: the hapten principle). In contrast the IgE epitopes of the major birch pollen allergen, Bet v 1, belong mainly to the conformational (discontinuous) type. In this case the allergen-IgE antibody interaction may be blocked with therapeutic allergen-specific antibodies which compete with patients IgE for the binding sites on the allergen and thereby prevent activation of effector cells. Such a therapeutic approach is reasonable especially when patients are sensitized to only a few major allergens.

In the present example 14 monoclonal Bet v 1 peptide-specific antibodies, all of them belonging to the IgG1 subclass, were characterized for their epitope-specific binding properties as well as for their capacity to interfere with allergic patient's IgE binding to the Bet v 1 allergen.

According to their binding specificities the monoclonal antibodies can be divided into two groups: Group I (clones 1-11) monoclonal antibodies strongly recognized peptide 2 (aa 30-59), whereas group II (clones 12-13) monoclonal antibodies strongly bound peptide 6 (aa 75-104). All monoclonal antibodies strongly bound rBet v 1 and rBet v 1 trimer and showed specificity because they failed to recognize unrelated control proteins such as KLH and rPhl p 1.

When tested for interference with binding of patients IgE to Bet v 1, each of the monoclonal antibodies inhibited IgE binding to a substantial degree, some of them up to 94% in certain patients. Interestingly, these results showed that a single monoclonal antibody is sufficient to compete with patients polyclonal IgE binding. Peptide 6-specific monoclonal antibodies showed stronger inhibition potency in comparison to peptide 2-specific monoclonal antibodies.

Comparing the inhibition potency of individual monoclonal IgG antibodies with different epitope specificities (clone 2: peptide 2-specific; clone 13: peptide 6-specific) with a mixture of two antibodies with different specificities, no stronger inhibition of patients IgE binding was observed with the antibody mixture.

Basically, two explanations for the blocking activity of the peptide-specific IgG1 antibodies may be considered. First, the inhibition may be explained by the fact that the obtained monoclonal antibodies recognize epitopes within or closely-related to the major IgE binding sites of Bet v 1. Second, the blocking activity may be caused by the modification of the conformation of the allergen so that the IgE epitopes or just a part of them are not longer accessible for IgE. The better explanation would fit to the results from the inhibition experiment showing that a mixture of two peptide-specific monoclonal antibodies, with a different epitope-specificity did not yield a stronger inhibition of IgE binding than the individual monoclonal antibodies. This theory may be confirmed by structural analyses of the allergen-antibody complex.

Several human and mouse monoclonal antibodies with therapeutical potential have already been isolated by classical tissue culture and combinatorial cloning technology using B cells from allergic patients or immunized mice as a source (Sun et al., 1995; Visco et al., 1996; Lebeque et al., 1997; Flicker et al., 2002). These antibodies were able to inhibit the allergen-IgE interaction and to prevent allergen-induced basophil degranulation.

Also Bet v 1-specific human blocking antibodies were already produced by the generation of hybridoma cell lines from patients treated by immunotherapy (Visco et al., 1996). Bet v 1-specific mouse monoclonal antibodies have been isolated by classical hybridoma technology (Lebeque et al., 1997). In comparison to the Bet v 1-specific monoclonal antibodies already obtained earlier, the antibodies described in this example have been isolated from mice, immunized with Bet v 1-derived peptides with a certain amino acid sequence, thereby determining the specific epitope already at the beginning of the procedure.

Blocking antibodies as described above may also be humanized or produced as recombinant antibody fragments to reduce their immunogenicity. Therapeutic allergen-specific antibodies may be administered locally into the target organs of allergy (e.g., nasal or bronchial mucosa, conjunctiva) to build up a stable defense line against intruding allergens or systemically such as passive vaccines (Valenta et al., 1997).

In conclusion, the monoclonal antibodies according to the present invention can also be used for the prevention of allergen-induced mediator release in the target organs of allergy by local therapy or passive vaccination.

### Example 2

A murine model to investigate the effects of passive vaccination with allergen-specific IgG antibodies in vivo was established (FIG. 1).

Mice (Charles River, Germany) were sensitized intraperitoneally (i.p.) with 5 μg rBet v 1 (Biomay, Austria), the major birch pollen allergen, adsorbed to Al(OH)₃ (Alu-Gel-S; Serva, Germany) on day 1, 14 and 28. Blood samples (ante-serum) were taken from the tail veins of the sensitized mice on day 36. Allergic sensitization to Bet v 1 was confirmed by the measurement of Bet v 1-specific IgE antibodies in these sera (Vrtala et al., 1998). The Bet v 1-specific IgE levels of all eight sera were comparable (Table 10, ante-serum).

Mice were then divided into two groups: Group 1 was treated i.p. with 0.5 ml Bet v 1-specific IgG. Group 2 (control group) was injected with 0.5 ml IgG directed against an unrelated allergen, Phl p 5. On day 37 blood was collected from the tail veins (post-serum) of mice from both groups and IgE reactivity to rBet v 1 was compared to that of ante-sera. For this purpose, 5 μg/ml rBet v 1 was coated overnight onto ELISA plates, plates were blocked with 3% BSA/TBST (50 mM Tris, 150 mM NaCl, 0.5% w/v BSA, 0.05% v/v Tween). Mouse sera were diluted 1:10 in TBST, incubated overnight and bound IgE was detected with a monoclonal rat-anti mouse IgE antibody (BD Pharmingen; USA) and a HRP-labelled goat anti-rat antiserum (Amersham, U.K.), respectively. Table 9 displays the results that represented means of duplicate determinations with variations of less than 10%. Column 1 and 2 show the IgE binding of mice to rBet v 1 before (ante-serum) and after (post-serum) treatment with IgG. The percentage inhibitions of IgE binding to rBet v 1 in post-sera (third column) were calculated as follows: Percentage inhibition=100−ODₚₒₛₜ₋ₛₑᵣᵤₘ×100/ODₐₙₜₑ₋ₛₑᵣᵤₘ. The inhibition rate ranged between 23.4-54.6% (Table 10).

Table 10 shows the inhibition of mouse IgE binding to rBet v 1 by rBet v 1-specific IgG antibodies. IgE binding to rBet v 1 is shown before (ante-serum; first column) and after (post-serum; second column) treatment with rBet v 1-specific IgG or Phl p 5-specific IgG. The percentages inhibition of IgE binding of postsera are displayed in the third column.

TABLE 10
Inhibition of mice IgE binding to rBet v 1.

| | OD_{405 nm} | | |
|---|---|---|---|
| | IgE-binding to rBet v 1 | | % inhibition of |
| Individual | ante-serum | post-serum | IgE binding to rBet v 1 |
| group 1 | | | |
| 1 | 1.110 | 0.850 | 23.4 |
| 2 | 0.738 | 0.335 | 54.6 |
| 3 | 0.417 | 0.220 | 47.2 |
| 4 | 0.362 | 0.197 | 45.6 |
| group 2 | | | |
| 5 | 1.884 | 1.784 | 5.3 |
| 6 | 0.508 | 0.503 | 1.0 |
| 7 | 0.200 | 0.175 | 12.5 |
| 8 | 0.468 | 0.497 | +6.2 |

Almost no inhibition of IgE binding to rBet v 1 was observed in mice of group 2 which had obtained IgG with specificity for Phl p 5. In a similar experiment carried out for Phl p 5 allergic mice, the percentage inhibition of IgE binding achieved by treatment with Phl p 5-specific IgG ranged from 7.7-58% (Table 11).

Table 11 shows the inhibition of mouse IgE binding to rPhl p 5 by rPhl p 5-specific IgG antibodies. IgE binding (OD levels) to rPhl p 5 is shown before (ante-serum; first column) and after (post-serum; second column) treatment with rPhl p 5-specific IgG or rBet v 1-specific IgG. The percentages inhibition of IgE binding of postsera are displayed in the third column.

TABLE 11
Inhibition of IgE binding to rPhl p 5.

| | OD_{405 nm} | | |
|---|---|---|---|
| | IgE-binding to rPhl p 5 | | % inhibition of |
| Individual | ante-serum | post-serum | IgE binding to rPhl p 5 |
| group 1 | | | |
| 1 | 0.701 | 0.647 | 7.7 |
| 2 | 0.680 | 0.387 | 43.1 |
| 3 | 0.802 | 0.416 | 48.1 |
| 4 | 0.669 | 0.281 | 58.0 |
| group 2 | | | |
| 5 | 0.687 | 0.711 | +3.5 |
| 6 | 0.828 | 0.773 | 6.7 |
| 7 | 0.944 | 0.809 | 14.4 |
| 8 | 0.828 | 0.756 | 8.7 |

Whether allergen-specific IgG antibodies can inhibit allergen-induced immediate allergic reactions was analyzed using the 8-hexosaminidase release assay from rat basophil leukemia (RBL) cells. RBL-2H3 cells (Eccleston et al., 1973) were plated in 96 well tissue culture plates (4×10⁴/well) and cultured for 24 hours at 37° C. in 5% CO₂. The cells were washed two times in Tyrode's Buffer (Sigma-Aldrich, Austria) (137 mM NaCl, 2.7 mM KCl, 0.5 mM MgCl₂, 1.8 mM CaCl₂, 0.4 mM NaH₂PO₄, 5.6 mM D-glucose, 12 mM NaHCO₃, 10 mM N-2-hydroxyethylpiperazine-N′-2-ethanesulfonic acid (HEPES) and 0.1% w/v bovine serum albumin, pH 7.2). Different concentrations of allergen (0.5 μg/ml; 0.1 μg/ml; 0.02 μg/ml) were incubated with mice ante-sera and post-sera (1:10 diluted in Tyrode's Buffer), respectively. The allergen/IgE and allergen/IgG complexes were exposed to RBL cells and incubated for 2 hours in a humidified atmosphere at 37° C. The level of β-hexosaminidase release was measured by fluorescence spectroscopy (CYTO FLUOR™ 2350, Millipore, USA). Results are expressed as percentages of total β-hexosaminidase released achieved by addition of 1% v/v Triton X-100. FIGS. 2 and 3 shows that the β-hexosaminidase release of RBL cells is lower when cells are incubated with allergen plus post-serum compared to allergen plus ante-serum.

In further experiments the concept of blocking antibodies was extended to another seasonal important allergen, the major grass pollen allergen Phl p 1, and also to perennial allergens, as Der p 2, a major allergen from house dust mite and Cyp c 1, the major fish allergen. Additionally, the long-term effects of a single IgG antibody injection on the allergen-specific mice IgE binding was investigated.

Table 12 shows the percentage inhibition of mouse IgE binding to rPhl p 1 by rPhl p 1-specific IgG antibodies followed for three weeks after IgG application. The inhibition rate of the post-sera ranged from 63.3-39.5%. In a similar experiment carried out for Der p 2 sensitized mice, the percentage inhibition achieved by treatment with rDer p 2-specific IgG ranged from 63.5-27.5% (Table 13). For rCyp c1 sensitized mice the inhibition rate of the specific IgE binding reached 59.8-36% (Table 14).

TABLE 12
Inhibition of mouse IgE binding to rPhl p 1 by
rPhl p 1-specific IgG antibodies.
The percentage of IgE binding of post-serum to Phl p 1 is
shown at different points of time after treatment with rPhl p 1-
specific IgG (group 1) or Bet v 1-specific IgG (group 2). The
IgE binding of ante-sera is calculated as 100% reaction.

| | % inhibition of IgE binding to rPhl p 1 after | | | | |
|---|---|---|---|---|---|
| mouse | 24 h | 72 h | 1 week | 2 weeks | 3 weeks |
| 1 | 63.3 | 44.5 | 29.8 | 39.1 | 39.5 |
| 2 | 48.3 | 46.2 | 45.6 | nd | 54.7 |

TABLE 13
Inhibition of mouse IgE binding to rDer p 2 by rDer p
2-specific IgG antibodies.
The percentage of IgE binding of post-serum to Der p 2 is
shown at different points of time after treatment with rDer p 2-
specific IgG (group 1) or Bet v 1-specific IgG (group 2). The
IgE binding of ante-sera is calculated as 100% reaction.

| | % inhibition of IgE binding to rDer p 2 after | | | | |
|---|---|---|---|---|---|
| mouse | 24 h | 72 h | 1 week | 2 weeks | 3 weeks |
| 1 | 50.9 | 52.2 | 75.7 | 43.3 | 27.5 |
| 2 | 63.5 | 50.8 | 36.6 | 34.3 | 48.3 |

TABLE 14
Inhibition of mouse IgE binding to rCyp c 1 by rCyp c
1-specific IgG antibodies.
The percentage of IgE binding of post-serum to Cyp c 1 is
shown at different points of time after treatment with rCyp c 1-
specific IgG (group 1) or Bet v 1-specific IgG (group 2). The
IgE binding of ante-sera is calculated as 100% reaction.

| | % inhibition of IgE binding to rCyp c 1 after | | | | |
|---|---|---|---|---|---|
| mouse | 24 h | 72 h | 1 week | 2 weeks | 3 weeks |
| 1 | 53.2 | 59.8 | 47.2 | 44.8 | 36.0 |

### Example 3

In order to obtain other Bet v 1-specific antibodies, patients whose IgE responses had exclusively been directed at the major birch pollen allergen Bet v 1 were identified. DNA sequences of the IgE variable regions were obtained from these patients applying reverse transcription and PCR using a family-specific primers (VH1-VH6) together with a primer located in the first constant epsilon region. In total 336 Bet v 1-specific heavy chain variable sequences of these allergic patients have been identified (FIG. 4) which recognize IgE epitopes of Bet v 1 and, hence, react as blocking antibodies.

Pollen Counts, Characterization of Allergic Subjects

Among fivehundred allergic subjects six individuals with exclusive allergic sensitization to birch pollen were identified using a multi-allergen test system (MAST CLA allergen-specific IgE assay, Hitachi Chemical Diagnostics) containing 46 allergen sources (Alder pollen, Almond, Alternaria, Apple, Aspergillus, Birch pollen, Carrot, Casein, Cat dander, Celery, Cladosporium, Cockroach, Codfish, Dermatophagoides farinae, Dermatophagoides pteronyssinus, Dog dander, Grass mix, Guinea pig dander, Hamster dander, Hazel pollen, Hazelnut, Horse dander, Juniper, Latex, Milk protein, Mugwort, Olive tree, Parietaria, Peach, Peanut, Penicillium, Pine mix, Plantain, Plume mix, Potato, Rabbit, Ragweed, Rye, Rye flour, Sesame, Shrimp, Soy bean, Tomato, Walnut, Wheat flour, whole Egg). Blood samples from the six allergic subjects were obtained in spring and summer 2002 and 2005. At each appointment, Bet v 1-specific IgE levels were quantified in plasma by CAP-RAST measurements (Phadia) and allergic symptoms and anti-allergic medication were recorded. None of the selected subjects had received any kind of allergen-specific immunotherapy.

Birch pollen exposure in the individuals living area was recorded as described in (Drachenberg K J et al., Allergy 56 (2001): 498-505).

Identification of Allergen-Specific IgE Antibodies

To specify the allergen profile of selected allergic subjects, inhibition experiments with recombinant Bet v 1 were performed. Recombinant Bet v 1, purchased from Biomay, was coupled to CNBr-activated sepharose 4B (GE Healthcare Bio-Sciences AB) in a concentration of five mg protein per ml medium according to the manufacturer's instructions. 1500 μl of plasma of the six allergic persons were incubated with 500 μl of allergen-coupled gel by end-over-end rotation overnight at 4° C. Serum was recovered by centrifugation (4° C., 5 min, 5000 g). IgE levels against food allergen mix (egg white, milk protein, codfish, wheat flour, peanut and soy bean) and respiratory mix (mugwort, birch pollen, parietaria, timothy grass and ribwort) as well as IgE levels against birch pollen extract and r Bet v1 were determined before and after depletion by CAP-RAST measurements (Phadia) (Eibensteiner P et al., Immunology 101 (2000): 112-9). Further experiments were performed with three subjects who reacted exclusively to Bet v 1 in birch pollen.

PBMC Isolation and RT-PCR Amplification of IgE Transcripts

Peripheral mononuclear cells were isolated by Ficoll density-gradient centrifugation at the time of serum collection. Total cellular RNA was isolated using the guanidine isothiocyanate method and CsCl gradient centrifugation.

IgE transcripts were generated by the Superscript™ One-Step RT-PCR with Platinum® Taq (Invitrogen) using VH1-VH6 family specific primers together with a primer specific for the first constant region of the IgE heavy chain (Table 15).

TABLE 15

| Name | Specificity | Sequence 5′-3′ |
|---|---|---|
| VH1 | hu VH1 gene family | GGA ATT CAC TCC CAG GTG |
| | | CAG CTG CTC GAG TCT GG |
| VH2 | hu VH2 gene family | GGA ATT CGT CCT GTC CCA |
| | | GGT CAA CTT ACT CGA GTC |
| | | TGG |
| VH3 | hu VH3 gene family | GGA ATT CGT CCA GGT GGA |
| | | GGT GCA GCT GCT CGA GTC |
| | | TGG |
| VH4 | hu VH4 gene family | GGA ATT CGT CCT GTC CCA |
| | | GGT GCA GCT GCT CGA GTC |
| | | GGG |
| VH5 | hu VH5 gene family | GGA ATT CGT CTG TGC CGA |
| | | GGT GCA GCT GCT CGA GCT |
| | | CGG |
| VH6 | hu VH6 gene family | GGA ATT CGT CCT GTC ACA |
| | | GGT ACA GCT GCT CGA GTC |
| | | AGG |
| IgEC1 | hu κ-chain first | GAG AGG AAT TCG CTA CTA |
| | constant region | GTT TTG TTG TCG ACC CAG TCT |
| | | GTG |

PCR amplification procedure consisted of an initial step of 30 min at 47° C. and 5 min at 94° followed by 40 cycles of 20 sec 94° C., 30 sec 59 and 1 min 72° C. with final extension of 5 min at 72° C. All PCR products of expected size were agarose gel purified using the Wizard® SV Gel and PCR Clean-Up System (Promega) according to the manufacturer's instructions. Subsequently, cDNA was cloned into the AccepTor™ Vector (Novagen) and transformed into Escherichia coli XL1-blue. Plasmid DNA was purified from 3 ml overnight culture containing 100 μg/ml ampicillin using Wizard® Plus SV Miniprep DNA Purification System (Promega) and digested with the restriction enzymes KpnI and SacI (Roche). Plasmids with inserts of the correct size were sequenced by Microsynth AG (Switzerland).

### REFERENCES

Ball T, et al. J. Biol. Chem. 1994; 269:28323-8.
Breiteneder H, et al. EMBO J. 1989; 8:1935-8.
Eccelston E, et al. Nat New Biol 1973; 244:73-76.
Ferreira F D, et al. J. Biol. Chem. 1993; 268:19574-80.
Flicker S, et al. Eur. J. Immunol. 2002; 32:2156-62.
Focke M, et al. Clin. Exp. Allergy. 2004; 34:1525-33.
Hoffmann-Sommergruber K, et al. Expr. Purif. 1997; 9:33-9.
Jarolim E, et al. Allergy. 1989; 44:385-95.
Kearney J F, et al. J. Immunol. 1979; 123:1548-50.
Köhler G, Milstein C. Nature 1975; 256:495.
Lebecque S, et al. J. Allergy Clin. Immunol. 1997; 99:374-84.
Sun L K, et al. Biotechnology. 1995; 13:779-86.
Valenta R, et al. J. Allergy. Clin. Immunol. 1991; 88:889-94.
Valenta R, et al. Curr. Opin. Immunol. 1995; 7:751-6.
Valenta R, et al. Immunol. Cell. Biol. 1996; 74:187-94.
Valenta R, et al. Biol. Chem. 1997; 378:745-9.
Valenta R, et al. Int. Arch. Allergy Immunol. 1998; 116:167-76.
Visco V, et al. J. Immunol. 1996; 157:956-62.
Vrtala S, et al. J. Immunol. 2000; 165:6653-9.
Vrtala S, et al. FASEB J. 2001; 15:2045-7.
Vrtala S, at al. J Immunol 1998; 60:6137-6144.

1. A method for the passive immunisation of allergic reactions in an individual comprising the steps of:
providing to an individual a composition comprising an antibody that binds to a Bet v 1 fragment comprising amino acids 30 to 59 (SEQ ID No. 1) or amino acids 75 to 104 (SEQ ID No. 2).

2. The method according to claim 1, wherein the antibody is an IgG antibody.

3. The method according to claim 1, wherein the antibody is a murine antibody.

4. The method according to claim 1, wherein the antibody is a chimeric antibody.

5. The method according to claim 1, wherein the antibody is humanized.

6. The method according to claim 1, wherein the antibody is a monoclonal antibody.

7. The method according to claim 1, wherein the antibody is a monoclonal antibody secreted by a hybridoma having assigned accession numbers DSM ACC2782, DSM ACC2783, DSM ACC2785, DSM ACC2784 or DSM ACC2786.

8. A composition comprising an Antibody or derivative thereof that binds to amino acids 30 to 59 (SEQ ID No. 1) or amino acids 75 to 104 (SEQ ID No. 2) of a Bet v 1 fragment.

9. The composition according to claim 8, wherein the antibody is an IgG antibody.

10. The composition according to claim 8, wherein the antibody is a murine antibody.

11. The composition according to claim 8, wherein the antibody is a chimeric antibody.

12. The composition according to claim 8, wherein the antibody is humanized.

13. The composition according to claim 8, wherein the antibody is a monoclonal antibody.

14. The composition according to claim 8, wherein the antibody is a monoclonal antibody secreted by a hybridoma.

15. A vaccine formulation comprising an antibody or derivative thereof that binds to amino acids 30 to 59 (SEQ ID No. 1) or amino acids 75 to 104 (SEQ ID No. 2) of a Bet v 1 fragment.

16. The vaccine according to claim 15, wherein the formulation is adapted for intramuscular, subcutaneous, intravenous or mucosal administration.

17. A composition comprising a nucleotide sequence selected from the group consisting of SEQ ID No. 3 to 298.

18. A polypeptide encoded by the nucleic acid molecule according to claim 17.

19. An antibody or fragment thereof comprising the polypeptide according to claim 18.

20. The antibody or fragment thereof according to claim 19, wherein the antibody is an immunoglobulin selected from the group consisting of IgG1, IgG2, IgG3 and IgG4.

21. The antibody or fragment thereof according to claim 18, wherein the antibody or fragment comprises a constant region of an immunoglobulin, a variable region of an immunoglobulin, single chain Fv (scFv), diabodies (dsFv), Fab or combinations thereof.

22. The method according to claim 1, wherein the antibody is an IgG1 or IgG4 isotype.

23. The composition according to claim 8, wherein the IgG antibody comprises an IgG1 isotype or an IgG4 isotype.

24. The composition according to claims 8, wherein the antibody is a monoclonal antibody secreted by a hybridoma comprising accession numbers selected from DSM ACC2782, DSM ACC2783, DSM ACC2785, DSM ACC2784 and DSM ACC2786.


SEQUENCE LISTING (298 sequences)

SEQ ID NO: 1 - protein, 31 aa, Artificial
  Bet v 1-Fragment
LFPKVAPQAISSVENIEGNGGPGTIKKISFC

SEQ ID NO: 2 - protein, 31 aa, Artificial
  Bet v 1-Fragment
CVDHTNFKYNYSVIEGGPIGDTLEKISNEIK

SEQ ID NO: 3 - DNA, 372 bp, Artificial
  DNA sequence coding for an IgE variable region
actcccaggtgcagctgctcgagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaacaaaacgtagtatagtgggagccactcaatttgactactggggccagggaaccctggtcaccgtctcctcag

SEQ ID NO: 4 - DNA, 381 bp, Artificial
  DNA sequence coding for an IgE variable region
actcccaggtgcagctgctcgagtctggcccaggactggtgacgccttcacagaccctgtccctcgcctgcactgtctctggtgtctccgtcaacagtggtggttactactggacctggctccgccagctcccagggaagggcctggagtatcttggatacatctattacactgggaacgcctactacaacccgtcgtttaagagtcgacttgccatttcaatagacacgtctaagaatcagttttcgctgaacctgacctctgtgactgccgcggacacggccgtatattactgtacgagagccccggcgccacacctatcctcccccccacaaggcttcgacccctggggccagggaaccctggtcaccgtctcctcag

SEQ ID NO: 5 - DNA, 390 bp, Artificial
  DNA sequence coding for an IgE variable region
actcccaggtgcagctgctcgagtctggcccaggactgctgaagccttcggagaccctgtccctcacctgcactgtctctgggggctccatcagtagtttctactggagctggatccggcagcccgccgggaagaagggcctggaatggattggccttatctatcccaggggcagcaccaactacaacccctccctcaagagtcgagtcaccatgtcagtggacacgtccaagaaccagttctccctgaatttaacttctgtgaccgccgcggacacggccgtgtattactgtgcgagaggtcgtcgcctcctcccaaccctgcgcatacaaccacctcttacgtggttcgacccctggggccagggaaccctggtcaccgtctcctctg

SEQ ID NO: 6 - DNA, 384 bp, Artificial
  DNA sequence coding for an IgE variable region
actcccaggtgcagctgctcgagtctggggctgaggtgaagaagcctggggcctcagtgaaggtctcctgcaaggcttctggatacaccttcatcggctactatatgcactgggtgcgacaggcccctggacaagggcttgagtggatgggatggatcaaccctaacaatgatgtcacacactatgcacagcagtttcagggcagggtcaccatgaccagggacacgtccatcagcacagcctacatggagttgagtaggctgagatctgacgacacggccgtatattactgtgcgagagataaccctgaccgatatagtgggaactactactactccggtttggacgtctggggccaagggaccacggtcaccgtctcctcag

SEQ ID NO: 7 - DNA, 381 bp, Artificial; identical to SEQ ID NO: 4
  DNA sequence coding for an IgE variable region

SEQ ID NO: 8 - DNA, 384 bp, Artificial; identical to SEQ ID NO: 6
  DNA sequence coding for an IgE variable region

SEQ ID NO: 9 - DNA, 369 bp, Artificial
  DNA sequence coding for an IgE variable region
actcccaggtgcagctgctcgagtctggagctgaggtgaagaagtctggggcctcagtaaaggtctcctgcaaggcttctggatacacctttaccagctacggtttcagctgggtgcgacaggcccctggacaggggcttgagtgggtgggatggatcagcggttccactggtaacacaaactatgcacagaaattccaagacagagtcgccatgaccatagatacatccacaaacacagcccacatggagctgaggagcctgagatctgacgacacggccgtttattattgtgcgagacgtaatggggcttactatctctggtattttgactactggggccagggaagcctggtcaccgtctcctcag

SEQ ID NO: 10 - DNA, 384 bp, Artificial; identical to SEQ ID NO: 6
  DNA sequence coding for an IgE variable region

SEQ ID NO: 11 - DNA, 369 bp, Artificial
  DNA sequence coding for an IgE variable region
actcccaggtgcagctgctcgagtctggggctgaggtgaagaagcctggatcctcggtgaaggtctcctgcaaggcctcaggaggcaccttcagcacctctgctatcagctgggtgcgacaggcccctggacaagggcttgagtggatgggagggatcatccctaagtttggaacatcaaactatgcacagaagttccagggcagagtcacgattaccgcggacgaatctacgagcacaggttacatggacctgagcagcctcacatctgaagacacggccgtctattactgtgcggctggcttttacgccagtggctggtatcgttacgagtcgtggggccagggaaccctggtcaccgtctcctcgg

SEQ ID NO: 12 - DNA, 376 bp, Artificial
  DNA sequence coding for an IgE variable region
acttccaggtgcagctgctcgagtctgggggagacttggtacagcctggggggtccctgagactctcctgtgcagcctctggattcagccttaacaactatgccatgagctgggtccgccaggctccagggaaggggctggagtgggtctcagctattagtggtagtggtggtagcacatactacgcagactccgtgaggggccggttctccatctccagagacaattccaagaccacgctgtatttgcagatagaacagcctgagagccgaggacacggccatttattattgtgtgagagatcccctctattacgatattttgactggctacttcgatctctggggccgtggcgccctggtcactgtctcctcag

SEQ ID NO: 13 - DNA, 369 bp, Artificial; identical to SEQ ID NO: 11
  DNA sequence coding for an IgE variable region

SEQ ID NO: 14 - DNA, 381 bp, Artificial
  DNA sequence coding for an IgE variable region
actcccaggtgcagctgctcgagtctggcccaggactggtgaagccttcacagaccctgtccgtcacatgcgctgtctctggtgcctctgtctctagaggtggttattactggacctggctccgccagccgccaggaaagggcctggagtggattggcgacatttattacactggggccacctactacaacccgtcactcaagcccagatttacgatgtcaataaacgcgtctaacaaccagttctccctgaaggtggcctctgtgactgtcgccgacacggccacttattattgtgcgagaatcccggcggcacacacgtcctccggcccacaaggcttcgacccctggggccagggaaccctggtcaccgtctcctcag

SEQ ID NO: 15 - DNA, 391 bp, Artificial
  DNA sequence coding for an IgE variable region
atctcccaggtgcagctgctcgagtctggcccaggactgctgaagccttcggagaccctgtccctcacctgcactgtctctgggggctccatcagtagtttctactggagctggatccggcagcccgccgggaagaagggcctggaatggattggccttatctatcccaggggcagcaccaactacaacccctccctcaagagtcgagtcaccatgtcagtggacacgtccaagaaccagttctccctgaatttaacttctgtgaccgccgcggacacggccgtgtattactgtgcgagaggtcgtcgcctcctcccaaccctgcgcatacaaccacctcttacgtggttcgacccctggggccagggaaccctggtcaccgtctcctctg

SEQ ID NO: 16 - DNA, 381 bp, Artificial; identical to SEQ ID NO: 14
  DNA sequence coding for an IgE variable region

SEQ ID NO: 17 - DNA, 381 bp, Artificial; identical to SEQ ID NO: 14
  DNA sequence coding for an IgE variable region

SEQ ID NO: 18 - DNA, 372 bp, Artificial
  DNA sequence coding for an IgE variable region
actcccaggtgcagctgctcgagtctgggggaggcttggtccagcctggggggtccctgagactctcctgtgcagcctctggattcacctttagtacctattggatgagctgggtccgccaggctccagggaaggggctggagtgggtggccaacataaagcgagatggaagtgagaaatactatgtggactctgtgaagggccgattcaccatctccagagacaacgccgagaactcactgtatctgcaaatgaacagcctgagagccgaggacacggctgtgtattactgtgcgaaagtgaggactctcctgagcgacgtgggcggattcgacccctggggccagggaaccctggtcaccgtctcctcag

SEQ ID NO: 19 - DNA, 387 bp, Artificial
  DNA sequence coding for an IgE variable region
actcccaggtgcagctgctcgagtctggcccaggactggtgaggccttcggggaccctgtccctcacctgcactgtctctgctgactccatcagcaattactactggacctggatccggcagcccccagggaagggactggagtggattggatatatctatgacagtgggagtagtaattacaacccctccctcaagagtcgagtcaccatgtcagtagacacgtccaagaaccagttctccctgacgctgcgttctgtgaccgctgcggacacggccatgtattactgtgcgagggggagacgatatagtgactacgatttgaacgtctactactactacgctatggacctctggggccaagggacttcggtcaccgtctccacag

SEQ ID NO: 20 - DNA, 381 bp, Artificial; identical to SEQ ID NO: 4
  DNA sequence coding for an IgE variable region

SEQ ID NO: 21 - DNA, 387 bp, Artificial
  DNA sequence coding for an IgE variable region
actcccaggtgcagctgctcgagtctggggctgagttgaagaagcctgggtcttcggtgaacgtctcctgcagggcttctgaagacaccttcaggagatatgtaataagttgggtccgacaggcccctggacaacggcctgagtggatgggagcgatcatccccatacttgattcgacaaaccaggcacggaaattccaggacagagtcacgattaccgcggacttatccacgagcacagcctacatgcaactgagcagcctgagatctgaagacacggccgtgtattactgtgcgagaggacttaggggggggcccctccatagcagtggttctcacaactactttgactcctggggccagggaaccctggtcaccgtctcctcag

SEQ ID NO: 22 - DNA, 384 bp, Artificial; identical to SEQ ID NO: 6
  DNA sequence coding for an IgE variable region

SEQ ID NO: 23 - DNA, 369 bp, Artificial; identical to SEQ ID NO: 11
  DNA sequence coding for an IgE variable region

SEQ ID NO: 24 - DNA, 378 bp, Artificial
  DNA sequence coding for an IgE variable region
actcccaggtgcagctgctcgagtctggggctgaggtgaagaagcccgggtcctcgatgagggtctcctgcagggcctctgaaggcaccttcagtacctatgttgtcagctgggtccgacaggcccctggacaagggcttgagtggatcggaactatcatccctatctttggaacaacaacttacgcacagaggttccagggcagactcacgcttagtgcggacgaatctctgagcacagtctatatggaggtgtccagcctcatgtctaatgacacggccgtgttcttctgcgcgagcaatcggcggagcaccggcattaaaataccgggggcttttgatacctggggccaagggacactggtcaccgtctcttcag

SEQ ID NO: 25 - DNA, 360 bp, Artificial
  DNA sequence coding for an IgE variable region
actcccaggtgcagctgctcgagtctggggctgaggggaagaagcctggggcctcagtgaacgtctcctgtcagacttctggatacaccttcaccgcctactatatacactgggtgcgacaggcccctggacaagggcttgagtggatggggtacatcaaccctcacactggtggcacacactctgcacagaagtttcagggctgggtcaccatgaccagggacacgtccatcagcacagcctacatggaaatgaagagactgaaatctgacgacgcggccgtgtattactgtgcgagagggggccgcggctggttccttgatcactggggccagggaaccctggtcaccgtgtcctcag

SEQ ID NO: 26 - DNA, 383 bp, Artificial
  DNA sequence coding for an IgE variable region
ctcccaggtgcagctgctcgagtctggggctgaggtgaagaagcctggggcctcagtgaaggtctcctgcaaggcttctggatacaccttcatcggctactatatgcactgggtgcgacaggcccctggacaagggcttgagtggatgggatggatcaaccctaacaatgatgtcacacactatgcacagcagtttcagggcagggtcaccatgaccagggacacgtccatcagcacagcctacatggagttgagtaggctgagatctgacgacacggccgtatattactgtgcgagagataaccctgaccgatatagtgggaactactactactccggtttggacgtctggggccaagggaccacggtcaccgtctcctcag

SEQ ID NO: 27 - DNA, 393 bp, Artificial
  DNA sequence coding for an IgE variable region
gtcaggtggaggtgcagctgctcgagtctgggggaggcttggtacagcctggggggtccctgagactctcctgtgcagcctctggattcacctttagtagctatgccatgacctgggtccgccagcctccagggaaggggctggagtgggtctcaactattagtggtggtggtgatagcacatactacgcagactccgtgaagggtcggttcaccatctccagagacaattccaagaacacactgtttctgcaagtgaacagcctgagagccgacgacacggccgtatattactgtgcgaaagatcgccacgtattgcctattatagtagtaccagttgctattgagtttctgggcgtctggggccaagggaccacggtcaccgcctcctcag

SEQ ID NO: 28 - DNA, 369 bp, Artificial
  DNA sequence coding for an IgE variable region
gatcccaggtgcagctgctcgagtctgggggaggcgtggtccagcctgggaggtccctgagactctcctgtgcagcgtctggattcaccttcagtagctttggcatgcactgggtccgccaggctccaggcaaggggctggagtgggtggcagttgtatggtatgagggaagtgataaatactatgcagactccgtggagggccgattcaccatctccagagacaattccaagaacatgctatatctgcaaatgaacaccctgagagccgacgacacggctgtctattactgtgcgagagatcgggggagtacagcagctcgtgcatttgactactggggccagggaaccctggtcaccgtctcctcag

SEQ ID NO: 29 - DNA, 369 bp, Artificial; identical to SEQ ID NO: 28
  DNA sequence coding for an IgE variable region

SEQ ID NO: 30 - DNA, 354 bp, Artificial
  DNA sequence coding for an IgE variable region
actcccaggtgcagctgctcgagtctggcccaggactggtgaagccttcggagacgctgtccctcacctgcactgtctcgggtgactccatgacgaccttctactgggcctggatccgccagccccccgggaagggactggagtggataggttatatatacgatgatggaagcaccaactccaacccctccctcaagagtcgagtcaccatatcagttgacacgtccaagaaccagttctccctgaatctgaggtctgtcaccgcggcggacacggccgtctattactgtgcgagagtaaatgggggatcctctgactactggggccagggaaccctggtcaccgtctcctcag

SEQ ID NO: 31 - DNA, 375 bp, Artificial
  DNA sequence coding for an IgE variable region
actcccaggtgcagctgctcgagtctgggggaggcttggtgcggcctggggggtccctgagactctcctgtgcggcctctggattcacctttagcaactttgccatgagctgggtccgccaggcgccagggaaggggctggagtgggtctcagcttttagtggcagtggaactaccacatactatgctgactccgtgaagggccgattcaccatcttcagagacaactcccggaacacgatatatttgcaaatgaacagcgtgagagccaaggacacggccgtgtactactgtgcgaaagcgaagacgatcttcggagtggttcgcgaaggctttgatcactggggccagggaaccctggtcaccgtctcctcag

SEQ ID NO: 32 - DNA, 378 bp, Artificial
  DNA sequence coding for an IgE variable region
actcccaggtgcagctgctcgagtctgggggaggcgtggtccaggctgggaggtccctgagactctcctgtgcagcgtctggattcagtttcagtagctatggcatgcactgggtccgccaggctccaggcaaggggctggagtgggtggcagttatatggtatgatggaagtaatagatactatgcagactccgtgaagggccgattcaccatctccagagacaattccaagaacacgctatatatgcagatgaacagcctgagagtcgacgacacggctgtgtattactgtgcgagagaccctttttaccgggcaagcagtggctggtacgactttgactcctggggccagggaaccctggtcaccgtctcctcag

SEQ ID NO: 33 - DNA, 369 bp, Artificial
  DNA sequence coding for an IgE variable region
actcccaggtgcagctgctcgagtctggcccaggactggtgaagccttcggagaccctgtccctcacctgcactgtctctggtggctccgtcagtagttactactggagctggatccggcagcccccagggaagggactggagtggattggacatatctattacggtgggaggagttactacaatccctccctccagagtcgagtcaccatatcagtagacacgtccaagaacgacttctccctgaacctgagctctgtgaccgctgcggacacggccgtttattactgtgcgaggctctgggtcggaggacactactactacggtatggacgtctggggccaagggaccacggtcaccgtctcatcag

SEQ ID NO: 34 - DNA, 363 bp, Artificial
  DNA sequence coding for an IgE variable region
actcccaggtgcagctgctcgagtctggggcggaggtgaagaagcctgggtcctcggtgaaggtctcctgcaaggccactggaggcaacttcagcagttccgctatcaactgggtgcgacaggcccctggacaagggctcgagtggatgggaggcttcatccatatctttggcacagcggtctacgcacagaagttccagggcagagtcacggttaccgcggacgaattcacgaccacagcctacatggagctgagcagcctgagatctgacgacacggccgtgtattattgtgcggggtggagagagtcatcgttggtctttgacctctggggccggggaaccctggtcaccgtctccgcgg

SEQ ID NO: 35 - DNA, 372 bp, Artificial
  DNA sequence coding for an IgE variable region
actcccaggtgcagctgctcgagtctgggggaggcttggtacagccgggggggtccctgagactctcctgtgcagcctctggattcacctttagcagctatgccatgagttgggtccgccaggctccagggaaggggctggagtgggtctcagggataactggaagtggaagtaccacaaactacgcagactccgtgaagggccggttcatcatctccagagacaattctaagaatacactatatctgcaaatgaacggcctgagagccgaggacacggccgtctattactgtgcgaaagcagatgttatggtggtcctaacttcttatatggacgtctggggccaggggaccacggtcaccgtctcctcag

SEQ ID NO: 36 - DNA, 370 bp, Artificial
  DNA sequence coding for an IgE variable region
gtccaggtggaggtgcagctgctcgagtctgggggaggcttggtacagccgggggggtccctgagactctcctgtgcagcctctggattcacctttagcagctttgccatgagctgggtccgccaggctccagggaaggggctggagtgggtctcagcaattggtggtagtggtgataccacatactacgcagactccgtgaagggccggttcaccatctccagagacaattccaagaacacgctgtatctgcaaatgaacagcctgagagccgaggacacggcctcatattactgtgcgaagttccagttggtacgctggggaaggttcgacccctggggccagggaaccctggtcaccgtctcctcag

SEQ ID NO: 37 - DNA, 260 bp, Artificial
  DNA sequence coding for an IgE variable region
actcccaggtgcagctgctcgagtctggggctgaggtgaagaagcctggggcctcagtgaaggtctcctggaagtcttctagatacaccttcaacgactactatataaacggggtgcgacaggcccctggataagggcctgagtggctgggacaagagattactttaattcagggagtttttagttcgcccgttgttgatacttccgctaataaaacatggacgtttggggcaaagggaccgcggtcaccgtctccgcag

SEQ ID NO: 38 - DNA, 376 bp, Artificial
  DNA sequence coding for an IgE variable region
gtccaggtggaggtgcagctgctcgagtctgggggcggcctggtcaagccgggggggtccctgagactctcctgtgcagcctctggattcaccttcagtagttatagcatgaactgggtccgccaggctccagggaaggggctggagtggctctcagccattagtagcagtagtacttacaaatactatgcagactcagtgaggggccgattcaccatctccagagacaacgccaagacgtccttgtatctgcaaatgaacagcttgagagccgaggacacggcactatattactgtgtgagaggccccctcgtagtggttccagttgctatgtctccctactggggccagggaaccctggtctccgtctcctcag

SEQ ID NO: 39 - DNA, 397 bp, Artificial
  DNA sequence coding for an IgE variable region
gtccaggtggaggtgcagctgctcgagtctgggggaggcttggtacagcctggggggtccctgagactctcctgtacagcctctggattcacctttagtaactatgccatgagttgggtccgccaggctccagggaaggggctggagtgggtctcagctattattagtggtggtgatagcacatactacgcagactccgtgaagggccggttcactatctccagagacaattccaagaacacgctgtatctgcaaatgaacagcctgagagccgaagacacggccatttattactgtgcgaactacgcaaaccgcttcctaggatattgtagtggtgataggtgctctgatggtttcgatatctggggccaagggacaatggtcaccgtctcttcag

SEQ ID NO: 40 - DNA, 376 bp, Artificial
  DNA sequence coding for an IgE variable region
gtccaggtggaggtgcagctgctcgagtctgggggaggcttggtccagcctggggggtccctgagactctcctgtgcagcctctggattcacctttagtagctattggatgagctgggtccgccaggctccagggaaggggctggagtgggtggccaacataaagaaagatggaagtgagaaatactatgtggactctgtgaagggccgattcaccatctccagagacaacgccaagaactcactgtatctgcaaatgaacagcctgaaagccgaggacacggctgtgtattactgtgcgagagtgaagactctcctgagcgacgtgggcgggttcgacccctggggccagggaaccctggtcaccgtctcctcag

SEQ ID NO: 41 - DNA, 391 bp, Artificial
  DNA sequence coding for an IgE variable region
gtctaggtggaggtgcagctgctcgagtctgggggaggcgtggtacagcctggagggtccctgagactctcctgcacagcctctggattcctcttcaggagttatgaaatgaattgggtccgccaggctccaaggaaggggctggagtgggtctcatacattagtggtggtggcggaaccgtttactacgcagactctgtgaagggccgattcaccatctccagagacaacacccagacttcactgtatctgcaaatgaacagcctgagagccgaggacacggctgtatattactgtgcgcgagaggagactggttactatgatggaactggtcactactacgatgtttttgatatgtggggccaagggacaatggtcaccgtctcttcag

SEQ ID NO: 42 - DNA, 376 bp, Artificial
  DNA sequence coding for an IgE variable region
gtccaggtggaggtgcagctgctcgagtctgggggaggcttggtccagcctggggggtccctgagactctcctgtgcagcctctggattcacctttagtacctattggatgagctgggtccgccaggctccagggaaggggctggagtgggtggccaacataaagcgagatggaagtgagaaatactatgtggactctgtgaagggccgattcaccatctccagagacaacgccgagaactcactgtatctgcaaatgaacagcctgagagccgaggacacggctgtgtattactgtgcgaaagtgaggactctcctgagcgacgtgggcggattcgacccctggggccagggaaccctggtcaccgtctcctcag

SEQ ID NO: 43 - DNA, 388 bp, Artificial
  DNA sequence coding for an IgE variable region
gtccaggtggaggtgcagctgctcgagtctgggggaggcttggtacagccgggggggtccctgagactctcctgcgttgcctctggattcacctttagtagccatgccatgaactgggtccgccaggctccagggaaggggctggagtgggtctcaggaataggcagtcttggtcatagtacatattacgcagactccgtgaagggccggtgcaccgtctccagagacaattccaagaatactctattcttgcaaatgaacagcctgcgacctgacgacacggccgtatattactgtgcgagagaccgagggtctccgagcgggcttctgagtgggagttacccctttgactattggggccagggaatcctggtcaccgtctcctcag

SEQ ID NO: 44 - DNA, 382 bp, Artificial
  DNA sequence coding for an IgE variable region
gtccaggtggaggtgcagctgctcgagtctgggggatacttggtacagcctggggggtccctgagagtctcttgtgcagcctctggattcaactttagcaactatgccatgagctgggtccgccaggctccagggaaggggctggagtgggtctcgggtattagtgggagtggcgctagcacatactacgcagactccgtgaagggccggttcaccatctccagagacaaatccaagaacacgctctatctccaaatgagcagcctgagagccgacgacacggccgtatattactgtgcgaagcaaacttcacatcatgtgtggtattccagtggcccgtctgactactggggccagggaaccctggtcaccgtctcctcag

SEQ ID NO: 45 - DNA, 378 bp, Artificial
  DNA sequence coding for an IgE variable region
aggtgcagctgctcgagtcctgggggaggcttggtacagccgggggggccctgagactctcctgcgttgcctctggattcacctttagtagccatgccatgaactgggtccgccaggctccagggaaggggctggagtgggtctcaggaataggcagtcttggtcatagtacatattacgcagactccgtgaagggccggtgcaccgtctccagagacaattccaagaatactctattcttgcaaatgaacagcctgcgacctgacgacacggccgtatattactgtgcgagagaccgagggtctccgagcgggcttctgagtgggagttacccctttgactattggggccagggaatcctggtcaccgtctcctcag

SEQ ID NO: 46 - DNA, 388 bp, Artificial
  DNA sequence coding for an IgE variable region
gtccaggtggaggtgcagctgctcgagtctgggggaggcttggtacagccgggggggtccctgagactctcctgcgttgcctctggattcacctttagtagccatgccatgaactgggtccgccaggctccagggaaggggctggagtgggtctcaggaataggcagtcttggtcatagtacatattacgcagactccgtgaagggccggtgcaccgtctccagagacaattccaagaatactctattcttgcaaatgaacagcctgcgacctgacgacacggccgtatattactgtgcgagagaccgaggctctccgagcgggcttctgagtgggagttacccctttgactattggggccagggaatcttggtcaccgtctcctcag

SEQ ID NO: 47 - DNA, 379 bp, Artificial
  DNA sequence coding for an IgE variable region
gttcaggtggaggtgcagctgctcgagtctgggggagacttggtacagcctggggggtccctgagactctcctgtgcagcctctggattcagccttaacaactatgccatgagctgggtccgccaggctccagggaaggggctggagtgggtctcagctattagtggtagtggtggtagcacatactacgcagactccgtgaggggccggttctccatctccagagacaattccaagaccacgctgtatttgcagatgaacagcctgagagccgaggacacggccatttattattgtgtgagagatcccctctattacgatattttgactggctacttcgatctctggggccgtggcgccctggtcactgtctcctcag

SEQ ID NO: 48 - DNA, 361 bp, Artificial
  DNA sequence coding for an IgE variable region
gtccaggtggaggtgcagctgctcgagtctgggggaggcctggtcaagcctggggggtccctgagactctcctgtgcagcctctggattcaccttcagtagctatagcatgaagtgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaagctacttaggtgcttttgatatctggggccaagggacaatggtcaccgtctcttcag

SEQ ID NO: 49 - DNA, 365 bp, Artificial
  DNA sequence coding for an IgE variable region
gtccaggtggaggtgcagctgctcgagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtcttggggggtgcaggtgtagggtactttgactactggggccagggaaccctggtcaccgtctcctcag

SEQ ID NO: 50 - DNA, 382 bp, Artificial
  DNA sequence coding for an IgE variable region
gtccaggtggaggtgcagctgctcgagtctgggggatacttggtacagcctggggggtccctgagagtctcctgtgcagcctctggattcaactttagcaactatgccatgagctgggtccgccaggctccagggaaggggctggagtgggtctcgggtatcagtgggagtggtgctagcacatactacgcagactccgtgaagggccggttcaccatctccagagacaaatccaagaacacgctgtatctccaaatgagcagcctgagagccgacgacacggccgtctattactgtgcgaaacagacttcacatcatgtgtggtattccagtggcccgtctgactactggggccggggaaccctggtcaccgtctcctcag

SEQ ID NO: 51 - DNA, 375 bp, Artificial
  DNA sequence coding for an IgE variable region
actcccaggtgcagctgctcgagtctggcccaggactggtgaagccttcggagaccctgtccctcacctgcactgtctctggtggctccatcagtacttacttttggagctggatccggcagcccccagggaagggactggagtgggttgggtatatctattacaatggggccaccaactacagcccctccctcaagagtcgagtcaccatttcactagacacgtccaagaaccagttctccctgaagctgaggtctgtgaccgctgcggacacggccgtttattactgtgcgagagatggacacagcagcagctggtacgacgctggcccctttgactactggggccagggagccctggtcaccgtctcctcag

SEQ ID NO: 52 - DNA, 360 bp, Artificial
  DNA sequence coding for an IgE variable region
actcccaggtgcagctgctcgagtctggggctgaggggaagaagcctggggcctcagtgaacgtctcctgtcagacttctggatacaccttctccgcctactatatacactgggtgcgacaggcccctggacaagggcttgagtggatggggtacatcaaccctcacactggtggcacacactctgcacagaagtttcagggctgggtcaccatgaccagggacacgtccatcagcacagcctacatggagatgaagagtctgaaatctgacgacgcggccgtttattactgtgcgagagggggccgcggctggttccttgatcactggggccagggaaccctggtcgccgtgtcgtcag

SEQ ID NO: 53 - DNA, 388 bp, Artificial
  DNA sequence coding for an IgE variable region
gtccaggtggaggtgcagctgctcgagtctgggggcgacttggtacagccgggggggtccctgagactctcttgtgtaggctctggattcagcttcaacaactatgccttcagctgggtccgccaggctccagggaagggcctggagtgggtctcactaattacgcgtcttggtcatagttcaaattacgcagactccgtgaagggccggttcaccatctccagggacaattccaagaccacggtgtctttgcaaatgaccagcctgagagccgatgacacggccgtctattactgtgcgagagatcgagggtccccgagcgggcttctgagtgggagttacccctttgacctctggggccagggaaccccggtcaccgtttcctcag

SEQ ID NO: 54 - DNA, 361 bp, Artificial
  DNA sequence coding for an IgE variable region
gtccaggtggagggtgcagctgctcgagtctgggggaggccggtcaagcctggggggtccctgagactctcctgtgcagcctctggattcaccttcagtagctatagcatgaagtgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgggtattactgtgcgagaagctacttaggtgcttttgatatctggggccaagggacaatggtcaccgtctcttcag

SEQ ID NO: 55 - DNA, 376 bp, Artificial
  DNA sequence coding for an IgE variable region
gtccaggtggaggtgcagctgctcgagtctgggggatcactggtacagccgggggggtccctgagagtctcctgtgcagcctctggattcagctttagcagctatgccatgaattgggtccgccaggctccagggaaggggctggagtgggtctcaggaattagtggtagtggtgggaacacaaattacgcagactccgtgaagggccggttcatcatctccagagacaatgcaaagaacacactagatctgcaaatgaacagcctgagcgccgaggacacggccgtatattattgtgcgaaaggagatgttatagtggtggaagagtcttatatggacgtttggggccaagggaccaccgtcaccgtctcctcag

SEQ ID NO: 56 - DNA, 370 bp, Artificial; identical to SEQ ID NO: 36
  DNA sequence coding for an IgE variable region

SEQ ID NO: 57 - DNA, 394 bp, Artificial
  DNA sequence coding for an IgE variable region
gtccaggtggaggtgcagctgctcgagtctgggggaggcttggtacagcctggggggtccctgagactctcctgtgcagcctctggattcacctttagtagctatgccatgacctgggtccgccagcctccagggaaggggctggagtgggtctcaactattagtggtggtggtgatagcacatactacgcagactccgtgaagggtcggttcaccatctccagagacaattccaagaacacactgtttctgcaagtgaacagcctgagagccgacgacacggccgtatattactgtgcgaaagatcgccacgtattgcctattatagtagtaccagttgctattgagtttctgggcgtctggggccaagggaccacggtcaccgcctcctcag

SEQ ID NO: 58 - DNA, 374 bp, Artificial
  DNA sequence coding for an IgE variable region
gtccaggtgggtgcagctgctcgagtctgggggatcactggtacagccgggggggtccctgagagtctcctgtgcagcctctggattcagctttagcagctatgccatgaattgggtccgccaggctccagggaaggggctggagtgggtctcaggaattagtggtagtggtgggaacacaaattacgcagactccgtgaagggccggttcatcatctccagagacaatgcaaagaacacactagatctgcaaatgaacagcctgagcgccgaggacacggccgtatattattgtgcgaaaggagatgttatagtggtggaagagtcttatatggacgtttggggccaagggaccaccgtcaccgtctcctcag

SEQ ID NO: 59 - DNA, 376 bp, Artificial
  DNA sequence coding for an IgE variable region
gtccaggtggaggtgcagctgctcgagtctgggggagaagtcatacagccgggggggtccctgagagtctcctgtgcagcctctggattcagctttagtaactatgccatgaatggggtccgccaggctccagggaagggtctggagtgggtcgcaggaattagtggtagtggcggtagcacaaactacgcagactccgtgaagggccggttcatcatctccagagacaatgcaaagaacacactaaatctgcaaatgaaaagcctgagcgccgaggacacggccgtatattattgtgcgaaaggagatgttatagtggtggaagagtcttatatggacgtgtggggccaagggaccaccgtcaccgtctcctcag

SEQ ID NO: 60 - DNA, 394 bp, Artificial
  DNA sequence coding for an IgE variable region
gtccaggtggaggtgcagctgctcgagtctgggggaggcttggtacagcctggggggtccctgagactctcctgtgcagcctctggattcacctttagcagctatgccatgacctgggtccgccagcctccagggaaggggctggagtgggtctcaactattagtggtggtggtgatagcacatactacgcagactccgtgaagggtcggttcaccatctccagagacaattccaagaacacactgtttctgcaagtgaacagcctgagagccgacgacacggccgtatattactgtgcgaaagatcgccacgtattgcctattatagtagtaccagttgctattgagtttctgggcgtctggggccaagggaccacggtcaccgtctcctcag

SEQ ID NO: 61 - DNA, 392 bp, Artificial
  DNA sequence coding for an IgE variable region
gtccaggtggaggtgcagcatgctcgagtctgggggaggcttggtgcggcctggggggtccctgagactctcctgtgcggcctctggattcacctttagcaactttgccatgagctgggtccgccaggcgccagggaaggggctggagtgggtctcagcttttagtggcagtggaactaccacatactatgctgactccgtgaagggccgattcaccatcttcagagacaactcccggaacacgatatatttgcaaatgaacagcgtgagagccgaggacacggccgtgtactactgtgcgaaagcgaagacgatcttcggagtggttcgcgaaggctttgatcactggggccagggaaccctggtcaccgtctcctcaggtgagtcctcag

SEQ ID NO: 62 - DNA, 373 bp, Artificial
  DNA sequence coding for an IgE variable region
gtccaggtggaggtgcagctgctcgagtctggcccaggactggtgaagcctttagagaccctgtccctcaactgcactgtctctggtgactccatcagcagtggcaatcactactggagttggttccgccagcccccggggaagggcctggagtggattggctacatcttttacaatggaatctcctactacaagccgtccctcaagactcgatttaccatatcagtagacacgtcgcagaaccagttctccctgaggctgacttctgtgaccgccgcagacacggccgtatattactgtgccagaacttactatggttcggggaggccatctgactactggggccagggagccctggtcagcgtctcctcag

SEQ ID NO: 63 - DNA, 373 bp, Artificial
  DNA sequence coding for an IgE variable region
gtccaggtggaggtgcagctgctcgagtctgggggaggcttggtacagccgggggggtccctgagactctcctgtgcagcctctggattcacctttagcagctatgtcatgagttgggtccgccaggctccagggaaggggctggagtgggtctccactgttagtggtagtgcttatggcgcataccacgcagactccgtgaagggccggttcaccatctccagagacaattccaagaacactctgtttctgcaaatgaacagcctgagagccgaggacacggccgtatattattgtgctaaaggggcgttccacggccccgtcgcgcacattgactgctggggccagggaacccaggtcaccgtctcctcag

SEQ ID NO: 64 - DNA, 381 bp, Artificial
  DNA sequence coding for an IgE variable region
gtccagggtgggaggtgcagctgctcgagtctgggggaggcttggtgcggcctggggggtccctgagactctcctgtgcggcctctggattcacctttagcaactttgccatgagctgggtccgccaggcgccagggaaggggctggagtgggtctcagcttttagtggcagtggaactaccacatactatgctgactccgtgaagggccgattcaccatcttcagagacaactcccggaacacgatatatttgcaaatgaacagcgtgagagccgaggacacggccgtgtactactgtgcgaaagcgaagacgatcttcggagtggttcgcgaaggctttgatcactggggccagggaaccctggtcaccgtctcctcag

SEQ ID NO: 65 - DNA, 373 bp, Artificial
  DNA sequence coding for an IgE variable region
gtccaggtggaggtgcagctgctcgagtctgggggaggcttggtacagccgggggggtccctgagactctcctgtgcagcctctggattcacctttagcagctatgccatgagttgggtccgccaggctccagggaaggggctggagtgggtctccactgtaagtggtagtgcttatggcacataccacgcagactccgtgaagggccggttcaccatctccagagacaattccaagaacactctgtttctgcaaatgaacagcctgagagccgacgacacggccgtatattattgtgctaaaggggcgttccacggccccgtcgcgcacattgactgctggggccagggaacccaggtcaccgtctcctcag

SEQ ID NO: 66 - DNA, 376 bp, Artificial
  DNA sequence coding for an IgE variable region
gtccaggtggaggtgcagctgctcgagtctgggggaggcttggtacagccgggggggtccctgagagtctcctgtgcagcctctggattcacgtttagcaactatgccatgagttgggtccgccaggctcccgggaaggggctggagtgggtctcagggataactggaagtggaagtaccacaaactacgcagactccgtgaagggccggttcatcatctccagagacaattctaagaatacactatatctgcaaatgaacggcctgagagccgaggacacggccgtctattactgtgcgaaagcagatgttatggtggtcctaacttcttatatggacgtctggggccaggggaccacggtcaccgtctcctcag

SEQ ID NO: 67 - DNA, 367 bp, Artificial
  DNA sequence coding for an IgE variable region
gtccaggtggaggtgcagctgctcgagtctgggggaggcttcgtacagcctggggggtccctgagactctcctgtgcaggctctggattcacctttagcacctatgccatgagctgggtccgccaggctccagggaaggggctggagtgggtggctcttgtatcatatgatggaatcaatacatactacgcagactccgtgaagggccgattcaccatctccagagacaattccaagaacagactgtttctacaaatgaacagcctgagagctgaggacacggctgtgtattactgtgcgagaggcgtggctcgtcaagctccaattgactattggggccagggaaccctggtcaccgtctcctcag

SEQ ID NO: 68 - DNA, 370 bp, Artificial
  DNA sequence coding for an IgE variable region
gtccaggtggaggtgcagctgctcgagtctgggggaggcttggtccagcctgggggggtccctgagactctcctgtgcagcctctgattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagggagagccgaccctatagcagcccctgtgactactggggccagggaaccctggtcaccgtctcctcag

SEQ ID NO: 69 - DNA, 364 bp, Artificial
  DNA sequence coding for an IgE variable region
gtccaggtggaggtgcagctgctcgagtctgggggaggcttggtacagccgggggggtccctgagactctcctgtgcagcctctggattcacctttagcagctatgtcatgagctgggtccgccaggctccagggaaggggctggagtgggtctcaggtattagtggtggtgggggtagcacatactacgcagactccgtgaagggccggttcaccatctccagagacaattccaagaacacgctgtatctgctaatgaacagcctgagagccgcggacacggccgtatattactgtgcgaaagagcctactactttgattccgcactgctggggccagggaaccctggtcaccgtctcctcag

SEQ ID NO: 70 - DNA, 364 bp, Artificial; identical to SEQ ID NO: 69
  DNA sequence coding for an IgE variable region

SEQ ID NO: 71 - DNA, 376 bp, Artificial
  DNA sequence coding for an IgE variable region
gtccaggtggaggtgcagctgctcgagtctgggggatcactggtacagccggggggggtccctgagagtctcctgtgcagcctctgattcagctttagcagctatgccatgaattgggtccgccaggctccagggaaggggctggagtgggtctcaggaattagtggtagtggtgggaacacaaattacgcagactccgtgaagggccggttcatcatctccagagacaatgcaaagaacacactagatctgcaaatgaacagcctgagcgccgaggacacggccgtatattattgtgcgaaaggagatgttatagtggtggaagagtcttatatggacgtttggggccaagggaccaccgtcaccgtctcctcag

SEQ ID NO: 72 - DNA, 370 bp, Artificial; identical to SEQ ID NO: 36
  DNA sequence coding for an IgE variable region

SEQ ID NO: 73 - DNA, 373 bp, Artificial
  DNA sequence coding for an IgE variable region
gtccaggtggaggtgcagctgctcgagtctgggggaggcttggtacagccggggggggtccctgagactctcctgtgcagcctctgattcacctttagcagctatgccatgagttgggtccgccaggctccagggaaggggctggagtgggtctccactgtaagtggtagtgcttatggcacataccacgcagactccgtgaagggccggttcaccatctccagagacaattccaagaacactctgtttctgcaaatgaacagcctgagagccgacgacacggccgtatattattgtgctaaaggggcgttccacggccccgtcgcgcacattgactgctggggccagggaacccaggtcaccgtctcctcag

SEQ ID NO: 74 - DNA, 373 bp, Artificial
  DNA sequence coding for an IgE variable region
gtccaggtggaggtgcagctgctcgagtctgggggaggcttggtacagcctgggggggtccctgagactctcctgtgcagctctggattcacctttagcagctatgccatgagctgggtccgccaggctccagggaaggggctggagtgggtctcaactgtaagtggtagtggttatggcacataccacgcagactccgtgaagggccggttcaccatctccagagacaattccaacaacacactgtttctgcaaatgaacagcctgagagccgaggacgcggccgtatattattgtgctaaaggggcgttccacggcccagtctcgcaggttgactactggggccagggaacccaggtcaccgtctcctcag

SEQ ID NO: 75 - DNA, 370 bp, Artificial
  DNA sequence coding for an IgE variable region
gtccaggtggaggtgcagctcgagtctgggggaggcttggtacagccgggggggtccctgagactctcctgtgcagcctctggattcacctttaacaactatgccatgagctgggtccgccaggctccagggaagggcctggagtgggtctcagaaattagtggcagtggtggtaggacatcctacgcagactccgtgaagggccggttcaccatctccagagacaattccaagaacactctgtttctgcaaatgaacagcctgagggccgaggacacggccgtatattattgtgctaaaggggcgttccacggccccgtcgcgcacattgactgctggggccagggaacccaggtcaccgtctcctcag

SEQ ID NO: 76 - DNA, 378 bp, Artificial
  DNA sequence coding for an IgE variable region
gtccaggtggaggtgcagctgctcagtctgggggagacttggtacagccgggggggtccctgagactctcctgtgcagcctctggattcacccttatcaactatgccatgacctgggtccgccaggctccagggaaggggctggagtgggtctcagctattagtaggagtggtggtagcacatactacgcagaccccgtgaagggccggttcaccatctccagagacaattccaagaacacgctgtatctgcaaatgaacagcctgagagccgaggacacggccgtttattactgtgcgaaagatcgatgtagtggctgcacctactattacggtatggacgtctggggccaagggaccacggtcaccgtctcctcag

SEQ ID NO: 77 - DNA, 364 bp, Artificial
  DNA sequence coding for an IgE variable region
gtccaggtggaggtgcagctgctcgagtctgggggaggcttggtacagcctggggggtccctgagactctcctgtgcagcctctggattcacctttagcagctatgtcatgagctgggtccgccaggctccagggaaggggctggagtgggtcccaggtattagtggtggtgggggtagcacatactacgcagactccgtgaagggccggttcaccatctccagagacaattccaagaacacgctgtatctgctaatgaacagcctgagagccgcggacacggccgtatattactgtgcgaaagagcctactactttgattccgcactgctggggccagggaaccctggtcaccgtctcctcag

SEQ ID NO: 78 - DNA, 391 bp, Artificial
  DNA sequence coding for an IgE variable region
gtccaggtggaggtgcagctgctcgagtctgggggaggcttggtgcggcctggggggtccctgagactctcctgtgcggcctctggattcacctttagcaactttgccatgagctgggtccgccaggcgccagggaaggggctggagtgggtctcagcttttagtggcagtggaactaccacatactatgctgactccgtgaagggccgattcaccatcttcagagacaactcccggaacacgatatatttgcaaatgaacagcgtgagagccgaggacacggccgtgtactactgtgcgaaagcgaagacgatcttcggagtggttcgcgaaggctttgatcactggggccagggaaccctggtcaccgtctcctcaggtgagtcctcag

SEQ ID NO: 79 - DNA, 376 bp, Artificial; identical to SEQ ID NO: 55
  DNA sequence coding for an IgE variable region

SEQ ID NO: 80 - DNA, 379 bp, Artificial
  DNA sequence coding for an IgE variable region
gtccaggtggaggtgcagctgctcgagtctgggggaggcttggtgcggcctggggggtccctgagactctcctgtgcggcctctggattcacctttagcaactttgccatgagctgggtccgccaggcgccagggaaggggctggagtgggtctcagcttttagtggcagtggaactaccacatactatgctgactccgtgaagggccgattcaccatcttcagagacaactcccggaacacgatatatttgcaaatgaacagcgtgagagccgaggacacggccgtgtactactgtgcgaaagcgaagacgatcttcggagtggttcgcgaaggctttgatcactggggccagggaaccctggtcaccgtctcctcag

SEQ ID NO: 81 - DNA, 397 bp, Artificial
  DNA sequence coding for an IgE variable region
gtccaggtggaggtgcagctgctcgagtctgggggaggctgggtagagcctggggggtccctgagactgtcctgtgaagtctctggtttcagctttagcaagtatgccatgaactgggtccgccaggctccagggaaggggctggagtgggtctcagttattagtaatagtggtgataccacgaagtgcgcagactcggtggagggccgattcaccgtctcgcgagacaatgctaagaacaccctgtatctgcaaatggacagcctgcgagccggggacacggccatatattattgtgcgaaagaattgagggcttccatttttggagatcgggggttttacaattactacattctggacgtctggggacaagggacgacggtcaccgtctcctcag

SEQ ID NO: 82 - DNA, 370 bp, Artificial
  DNA sequence coding for an IgE variable region
gtccaggtggaggtgcagctgctcgagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagggagagccgaccctatagcagcccctgtgactactggggccagggaaccctggtcaccgtctcctcag

SEQ ID NO: 83 - DNA, 403 bp, Artificial
  DNA sequence coding for an IgE variable region
gtccaggtggaggtgcagctgctcgagtctgggggaggcttaatatagcctggggggtccctgagactctcctgtgcagcctctggattcacctttagtaactttgccatgaactgggtccgccaggctccagggaaggggccggagtgggtcgcgtccattagtactagtagtaattacatatattacgcagagtcagtgaagggccgattcaccatctccagagacaacgccaagaactcactgtttctgcaaatgaacagcctcagagccgaggacacggctcttttttactgtgcgagagatcgaaccccaacctattatgattacgtttgggggacttaccagacccgggatcctttcgatatctggggccaagggacaatggtcaccgtctcttcag

SEQ ID NO: 84 - DNA, 379 bp, Artificial; identical to SEQ ID NO: 80
  DNA sequence coding for an IgE variable region

SEQ ID NO: 85 - DNA, 376 bp, Artificial
  DNA sequence coding for an IgE variable region
gtccaggtggaggtgcagctgctcgagtctgggggaggcgtggtccagcctgggaggtccctgagactctcctgtgcagcctctggattcaccttcagtagctatcctatgcactgggtccgccaggctccaggcaaggggctggagtgggtggcccttatctcatatgatggaagcactaaatactacgcagactccgtgaagggccgattcaccatctccagagacaattccgagaacacactgtatctccaaatgaacagcctgaggggtgaggacacggctgtgtattactgtgcgagagagaggcaggagtgggagctgcacaactgcttcgacccctggggccagggaaccctggtcaccgtctcctcag

SEQ ID NO: 86 - DNA, 364 bp, Artificial
  DNA sequence coding for an IgE variable region
gtccaggtggaggtgcagctgctcgagtctggggctgaggggaagaagcctggggcctcagtgaacgtctcctgtcagacttctggatacaccttcaccgcctactatatacactgggtgcgacaggcccctggacaagggcttgagtggatggggtacatcaaccctcacactggtggcacacactctgcacagaagtttcagggctgggtcaccatgaccagggacacgtccatcagcacagcctacatggaaatgaagagactgaaatctgacgacgcggccgtgtattactgtgcgagagggggccgcggctggttccttgatcactggggccagggaaccctggtcaccgtgtcctcag

SEQ ID NO: 87 - DNA, 364 bp, Artificial; identical to SEQ ID NO: 86
  DNA sequence coding for an IgE variable region

SEQ ID NO: 88 - DNA, 376 bp, Artificial; identical to SEQ ID NO: 85
  DNA sequence coding for an IgE variable region

SEQ ID NO: 89 - DNA, 376 bp, Artificial
  DNA sequence coding for an IgE variable region
gtccaggtggaggtgcagctgctcgagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagaactggttggctactactacttctacggtatggacgtctggggccaagggaccacggtcaccgtctcctcag

SEQ ID NO: 90 - DNA, 391 bp, Artificial
  DNA sequence coding for an IgE variable region
gtccaggtggaggtgcagctgctcgagtctgggggaggcttggtacagcctggagggtccctgagactctcctgcacagcctctggattcctcttcaggagttatgaaatgaattgggtccgccaggctccagggaaggggctggagtgggtctcatacattagtggtggtggcggaaccgtttactacgcagactctgtgaagggccgattcaccatctccagagacaacacccagacttcactgtatctgcaaatgaacagcctgagagccgaggacacggctgtatattactgtgcgcgagaggagactggttactatgatggaactggtcactactacgatgtttttgatatgtggggccaagggacaatggtcaccgtctcttcag

SEQ ID NO: 91 - DNA, 385 bp, Artificial
  DNA sequence coding for an IgE variable region
gtccaggtggaggtgcagctgctcgagtctggcccaggactggtgaagccttcacagaccctgtccgtcacatgcgctgtctctggtgcctctgtctctagaggtggttattactggacctggctccgccagccgccaggaaagggcctggagtggattggcgacatttattacactggggccacctactacaacccgtcactcaagcccagatttacgatgtcaataaacgcgtctaacaaccagttctccctgaaggtggcctctgtgactgtcgccgacacggccacttattattgtgcgagaatcccggcggcacacacgtcctccggcccacaaggcttcgacccctggggccagggaaccctggtcaccgtctcctcag

SEQ ID NO: 92 - DNA, 388 bp, Artificial
  DNA sequence coding for an IgE variable region
gtccaggtggaggtgcagctgctcgagtctgggggaggcttggtacagccgggggggtccctgagactctcctgcgttgcctctggattcacgtttagtagtcatgccatgaactgggtccgccaggctccagggaaggggctggagtgggtctcaggaataggcagtcttggtcatagtacatattacgcagactccgtgaagggccggtgcaccgtctccagagacaattccaagaatactctattcttgcaaatgaacagcctgcgacctgacgacacggccgtatatttctgtgcgagagaccgagggtctccgagcgggcttctgagtgggagttacccctttgactattggggccagggaatcctggtcaccgtctcctcag

SEQ ID NO: 93 - DNA, 415 bp, Artificial
  DNA sequence coding for an IgE variable region
gtccaggtggaggtgcagctgctcgagtctgggggaggcttggtaaagcctggggggtcccttagactctcctgtgcagcctctggattcactttcaataacgcttgcatgggctgggtccgccaggctccagggaaggggctggagtgggttggccgtattaaaagcaaatctgatggtgggactaaaagcaaaactgatggtgggacagcagactacgctgcacccgtgaaaggcagattcaccatttcaagagatgattcaaaaaacacgctgtatctgcaaatgaacagcctgaaaaccgaggacacagccgtgtattattgtatcacagaccacatagcgaatagtgggaactacctcaagaggcgcattgactattggggccagggaaccctggtcaccgtctcctcag

SEQ ID NO: 94 - DNA, 385 bp, Artificial
  DNA sequence coding for an IgE variable region
gtccaggtggaggtgcagctgctcgagtctggcccaggactggtgaagccttcacagaccctgtccctcacctgcacagtctctggtgactccgtcagcagtggtgattacaactggagttggatccgtcaggccccagggaagggcctggagtggattgggcacatctatcacagtgggagcacctactacaacccgtccctcaagagtcgtcttgtcatgtcagtggacacgaccaagaggcagttctccctgaagctacactccgtgactgccgcagacacggccgtctatttctgtgccagagtggcagccttgtggtacggggacttagtgaatgcttttgatctttggggccaagggacaatggtcatcgtctcttcag

SEQ ID NO: 95 - DNA, 373 bp, Artificial
  DNA sequence coding for an IgE variable region
gtccaggtggaggtgcagctgctcgagtctggggctgaggtgaagaagcctggatcctcggtgaaggtctcctgcaaggcctcaggaggcaccttcagcacctctgctatcagctgggtgcgacaggcccctggacaagggcttgagtggatgggagggatcatccctaagtttggaacatcaaactatgcacagaagttccagggcagagtcacgattaccgcggacgaatctacgagcacaggttacatggacctgagcagcctcacatctgaagacacggccgtctattactgtgcggctggcttttacgccagtggctggtatcgttacgagtcgtggggccagggaaccctggtcaccgcctcctcgg

SEQ ID NO: 96 - DNA, 415 bp, Artificial; identical to SEQ ID NO: 93
  DNA sequence coding for an IgE variable region

SEQ ID NO: 97 - DNA, 382 bp, Artificial
  DNA sequence coding for an IgE variable region
gtccaggtggaggtgcagctgctcgagtctgggggatacttggtacagcctggggggtccctgagagtctcctgtgcagcctctggattcaactttagcaactatgccatgagctgggtccgccaggctccagggagggggctggagtgggtctcgggtattagtggtagtggtgctagcacatactacgcagactccgtgaagggccggttcaccatctccagagacaaatccaagaacacgctgtatctccaaatgagcagcctgagagccgacgacacggccgtatattactgtgcgaaacagacttcacatcatgtgtggtattccagtggcccgtctgactactggggccagggaaccctggtcaccgtctcctcag

SEQ ID NO: 98 - DNA, 373 bp, Artificial
  DNA sequence coding for an IgE variable region
gtccaggtggaggtgcagctgctcgagtctggagctgaggtgaagaagtctggggcctcagtaaaggtctcctgcaaggcttctggatacacctttaccagctacggtttcagctgggtgcgacaggcccctggacaggggcttgagtgggtgggatggatcagcggttccactggtaacacaaactatgcacagaaattccaagacagagtcgccatgaccatagatacatccacaaacacagcccacatggagctgaggagcctgagatctgacgacacggccgtttattattgtgcgagacgtaatggggcttactatctctggtattttgactactggggccagggaagcctggtcaccgtctcctcag

SEQ ID NO: 99 - DNA, 400 bp, Artificial
  DNA sequence coding for an IgE variable region
gtccaggtggaggtgcagctgctcgaggctggcccaggactggtgaagccttcggataccctgtccctcacctgcagtgtctctggtgggtccatcagcggtagcacttattactgggcctggatccgccagcccccgggaaaggggctgaattggattggtagtgtctattatagtggaagcacctactacaacccgtccctcaagagtcgagtcaccatatccgtagacacgtccaagaaccaattctccctgcacctgacctctgtgaccgccgcagacacggctgcgtatttctgtgcgagatctttaagatattactatgatagtagtgactctctgaccgaaaatgatgcttttgagatctggggccgagggacaatggtcaccgtctctgcag

SEQ ID NO: 100 - DNA, 379 bp, Artificial
  DNA sequence coding for an IgE variable region
gtcctgtcccaggtgcagctgctcgagtcgggccccggactggtgaagccttcacagaccgtgtccctcacctgcagtgtctctggtgactccatcagcagtggttacaactactggagttggatccggcagaccgccgggaagggactggagtggctcgggcgtatgtataccaatgggaacaccacacagtccatttactatcacccctccctcaagagtcgagtcagcatgtcagtagacacgtccaagaaccagatctccctgcaattgacgtctgtgactgccgcagacacggccgtctattactgtgcgagagatcggggagccatcgaccattactactggggccggggaacccaggtcaccgtctcctcag

SEQ ID NO: 101 - DNA, 390 bp, Artificial
  DNA sequence coding for an IgE variable region
gtcccccaggtgcagctgctcgagtcgggcccaggactggtgaagccttcacagaccctgtccctcacctgcagtgtctctgatggctccttcagcagtggtggtaactattggacctggacccgccagcacccagggaagggcctggagtggattgggttcacctattacattgggagcacctactacaacccgtccctcaagagtcgaacaaccatatcactagacacgtctaagaaccgcttctccctgaagctgacctctgtgactgccgcggacacgtccgtcttttactgtgcgagacatttttggctcgtaaccgacttcacctcataacggacggctggttcgacccctggggccagggaatcctggtcaccgtcccctcag

SEQ ID NO: 102 - DNA, 385 bp, Artificial
  DNA sequence coding for an IgE variable region
gtcctgtcccaggtgcagctgctcgagtcgggcccaggactggtgaagccttcacagaccctgtccgtcacatgcgctgtctctggtgcctctgtctctagaggtggttattactggacctggctccgccagccgccaggaaagggcctggagtggattggcgacatttattacactggggccacctactacaacccgtcactcaagcccagatttacgatgtcaataaacgcgtctaacaaccagttctccctgaaggtggcctctgtgactgtcgccgacacggccacttattattgtgcgagaatcccggcggcacacacgtcctccggcccacaaggcttcgacccctggggccagggaaccctggtcaccgtctcctcag

SEQ ID NO: 103 - DNA, 391 bp, Artificial
  DNA sequence coding for an IgE variable region
gtcctgtcccaggtgcagctgctcgagtcgggcccaggactggtgaggccttcggggaccctgtccctcacctgcactgtctctgctgactccatcagcaattactactggacctggatccggcagcccccagggaagggactggagtggattggatatatctatgacagtgggagtagtaattacaacccctccctcaagagtcgagtcaccatgtcagtagacacgtccaagaaccagttctccctgacgctgcgttctgtgaccgctgcggacacggccatgtattactgtgcgagggggagacgatatagtgactacgatttgaacgtctactactactacgctatggacctctggggccaagggacttcggtcaccgtctccacag

SEQ ID NO: 104 - DNA, 385 bp, Artificial
  DNA sequence coding for an IgE variable region
gtcctgtcccaggtgcagctgctcgagtcgggcccaggcctggtgaagccttcggggaccctgtccctcacctgcgctgtctctggtggctccatcaatagtactaaatggtggagttgggtccgccaggccccagggaaggggctggagtggattggggaaatctatcatagtgggaacaccaactacaacccgtccctcaagagtcgagtcaccatatcagtagacaagtccaagaaccagttctccctgaagatgacctctgtgaccgccgcggacgcggccgtatattactgtgcgagagttccccgtattacgctttttggagtgattagccccggtatggacgtctggggccaagggaccacggtcaccgtctcctcag

SEQ ID NO: 105 - DNA, 400 bp, Artificial
  DNA sequence coding for an IgE variable region
gtcctgtcccaggtgcagctgctcgagtcgggcccaggactggtgaagncttcngntaccctgtccctcacctgcagtgtctctggngggtccatcagcggtagcacttattactgggcctggatccgccagcccccgggaaaggggctgaattggattggtagtgtctattatagngnaagcacctactacaacccgtccctcaagagtcgagtcaccatatccgtagacacgtncaagaaccaattctccctgcacctgacctctgtgaccgccgcagacacggctgcgtatttctgtgcgagatctttaagatattactatgatagnagtgactctctgaccgaaaatgatgcttttgagatctggggccgagggacaatggtcaccgtctctgcag

SEQ ID NO: 106 - DNA, 367 bp, Artificial
  DNA sequence coding for an IgE variable region
gtcctgtcccaggtgcagctgctcgagtcgggcccaggactggcgaagccttcacagaccctgtccctcacctgcactgtctctggtggctccatcagcagtggtgattactactggagttggatccgccagtccccagggaagggcctggagtggattggatacctcttttatagtgggggcacctactacagcccgaccctcaagagtcgagttaccatatcagttgacacgtccaagaaccagttctccctgaacgtgacctctctgactgccgcagacacggccgtgtattactgtgccagagggggctgggatgatgcctttgatatctggggtcaagggactttggtcaccgtctcttcag

SEQ ID NO: 107 - DNA, 367 bp, Artificial
  DNA sequence coding for an IgE variable region
gtcctgtcccaggtgcagctgctcgagtcgggcccaggactggtgaagccttcggagactctgtccctcacctgcactatctctggtggctccatcagtacttacttctggagctggatccggcagcccccagggaagggacttgagtggattggatatatctatcacagtgggacccccaactacaacccctccctcaagagtcgagtcaccatatcagtggacacgtccaagaaccacttctccctgaaccttacctctgtgacctctgcggacacggccatgtattactgtgcgagagggggtctaaagtatagtagacgatttgactcctggggccagggaaccctggtcaccgtctcctcag

SEQ ID NO: 108 - DNA, 385 bp, Artificial
  DNA sequence coding for an IgE variable region
gtcctgtcccaggtgcagctgctcgagtcgggcccaggactggtgaagccttcacagaccctgtccctcacctgcacagtctctggtgactccgtcagcagtggtgattacaactggagttggatccgtcaggccccagggaagggcctggagtggattgggcacatctatcacagtgggagcacctactacaacccgtccctcaagagtcgtcttgtcatgtcagtggacacgaccaagaggcagttctccctgaagctacactccgtgactgccgcagacacggccgtctatttctgtgccagagtggcagccttgtggtacggggacttagtgaatgcttttgatctttggggccaagggacaatggtcatcgtctcttcag

SEQ ID NO: 109 - DNA, 379 bp, Artificial
  DNA sequence coding for an IgE variable region
gtcctgtcccaggtgcagctgctcgagtcgggcccaggactggtgaagccctcacagaccctgtctctcatgtgcaatgtctctggtggttccatcaacagtggtgattactattggaattggatccgccaccacccagggaagggcctggagtggattgggtacatccatcacagtgggagcacctactacagcccgtccctcaagggtcgagttaccatatcagttgccatgtccaagaaccagttttccctgaagctgacttctgtgactgtcgcagacacggccgtgtattactgtgccagaggggggccaggggcattaaacagtgggagctattggatctggggccaagggacaatggtcaccgtctcttcag

SEQ ID NO: 110 - DNA, 356 bp, Artificial
  DNA sequence coding for an IgE variable region
cccaggactggtgaagccttcacagaccctgtccctcacctgcactgtcgctggttactctatcagcagtggtcgttattactggacttggatccggcagtccgccagtaagggactggagtggattggccgtatccatatcagtggggacacaaattacaatccctccttcaagagtcgagtcaccatgtcagaggacatgtccaagaaccagttctccctgaagctgaggtctgtgaccgccgcagacacggccgtgtatttttgtgcgcgagtccgggacgacccagagcgggccacaattcagtatgcttttgatatctggggccaagggacaatggttaccgtgtcctcag

SEQ ID NO: 111 - DNA, 379 bp, Artificial
  DNA sequence coding for an IgE variable region
gtcctgtcccaggtgcagctgctcgagtcgggncccggactggtgaagccttcacagaccgtgtccctcacctgcgctgtctctggtgactccatcagtagtggttacaactactggagctggatccgccagcccgccgggaagggactggagtggatcgggcgtatgtataccagtgggagcaccggtgggaacatttattaccacccctccctcaagagtcgagtcaacatgtcagtggacacgtccaagaatcagatttccctggtattaacgtctgtcactgtcgcagacacggccgtctattactgtgcgagagatcggggagccatcgaccattactactggggccggggaaccctggtcaccgtctcctcag

SEQ ID NO: 112 - DNA, 379 bp, Artificial
  DNA sequence coding for an IgE variable region
gtcctgtcccaggtgcagctgctcgagtcnggnccaggactggtgaagccttcagagaccgtgtccctcacctgcactgtctctggggactccatcaacagtgcctacaactactggagctggatccgacagcccgccgggaagggactggagtggattgggcgtatgtacaccaatgggggcaccggtgggaacaccttctaccacccctccctcaagagtcgactcaccatgtcagtggacacgtccaagaaccatttctccctgaagctcaactctgtgaccgccgcggacacggccatatattactgtgcgcgagatcggggagccatcgaccattactactggggccggggaaccctggtcaccgtctcgtcag

SEQ ID NO: 113 - DNA, 379 bp, Artificial
  DNA sequence coding for an IgE variable region
gtcctgtcccaggtgcagctgctcgagtcgggccccggactggtgaagccttcacagaccgtgtccctcacctgcgctgtctctggtgactccatcagtagtggttacaactactggagctggatccgccagcccgccgggaagggactggagtggatcgggcgtatgtataccagtgggagcaccggtgggaacatttattaccacccctccctcaagagtcgagtcaacatgtcagtggacacgtccaagaatcagatttccctggtattaacgtctgtcactgtcgcagacacggccgtctattactgtgcgagagatcggggagccatcgaccattactactggggccggggaaccctggtcaccgtctcctcag

SEQ ID NO: 114 - DNA, 388 bp, Artificial
  DNA sequence coding for an IgE variable region
gtcctgtcccaggtgcagctgctcgagtcgggcccaggactggtgaagccttcacagaccctgtccctcacctgcactgtcgctggttactctatcagcagtggtcgttattactggacttggatccggcagtccgccagtaagggactggagtggattggccgtatccatatcagtggggacacaaattacaatccctccttcaagagtcgggtcaccatgtcagaggacatgtccaagaaccagttctccctgaagctgaggtctgtgaccgccgcagacacggccgtgtatttttgtgcgcgagtccgggacgacccagagcgggccacaattcagtatgcttttgatatctggggccaagggacaatggttaccgtgtcctcag

SEQ ID NO: 115 - DNA, 388 bp, Artificial
  DNA sequence coding for an IgE variable region
gtcctgtcccaggtgcagctgctcgagtcgggcccaggactggcgaagccttcacagaccctgtccctcacctgcactgtcgctggttactctatcagcagtggtcgttattactggacttggatccggcagtccgccagtaagggactggagtggattggccgtatccatatcagtggggacacaaattacaatccctccttcaagagtcgagtcaccatgtcagaggacatgtccaagaaccagttctccctgaagctgaggtctgtgaccgccgcagacacggccgtgtatttttgtgcgcgagtccgggacgacccagagcgggccacaattcagtatgcttttgatatctggggccaagggacaatggttaccgtgtcctcag

SEQ ID NO: 116 - DNA, 391 bp, Artificial
  DNA sequence coding for an IgE variable region
gtcctgtcccaggtgcagctgctcgagtcgggcccaggactggtgaggccttcggggaccctgtccctcacctgcactgtctctgttgactccatgagcagtttctattggacctggatccggcagcccccagggaagggactggagtggattggatatatctatgacagtggaagtaacaattacaacccctccctcaagagtcgagtcagcatgtcagtagacacgtccaagaaccaattctccctgacgctgcgttctgtgaccgctgcggacacggccatctattactgtgcgagggggagacgatatagtggctacgatttgaacgtctactactactacgctatggacctctggggccaagggacttcggtcatcgtctccacag

SEQ ID NO: 117 - DNA, 379 bp, Artificial
  DNA sequence coding for an IgE variable region
gtcctgtcccaggtgcagctgctcgagtcgggcccaggactggtgaagccttcagagaccgtgtccctcacctgcactgtctctggggactccatcaacagtgcctacaactactggagctggatccgacagcccgccgggaagggactggagtggattgggcgtatgtacaccaatgggggctccggtgggaacaccttctaccacccctccctcaagagtcgagtaaccatgtcagtggacacgtccaagaaccagttctccctgaagctcaactctgtgaccgccgcggacacggccatatattactgtgcgcgagatcggggagccatcgaccattactactggggccggggaaccctggtcaccgtctcgtcag

SEQ ID NO: 118 - DNA, 400 bp, Artificial
  DNA sequence coding for an IgE variable region
gtcctgtcccaggtgcagctgctcgagtcgggcccaggactggtgaagccttcggataccctgtccctcacctgcagtgtctctggtgcgtccatcagcggcagcacttattactgggcctggatccgccagcccccgggaaaggggctgaattggattggtagtgtctattatagtggaagcacctactacaacccgtccctcaagagtcgagtcaccatatccgtagacacgtccaagaaccaattctccctccacctgacctctgtgaccgccgcagacacggctgcgtatttctgtgcgagatctttaagatattactatgatagtagtgactctctgaccgaaaatgatgcttttgagatctggggccgaggaacaatggtcaccgtctctgcag

SEQ ID NO: 119 - DNA, 400 bp, Artificial; identical to SEQ ID NO: 118
  DNA sequence coding for an IgE variable region

SEQ ID NO: 120 - DNA, 379 bp, Artificial
  DNA sequence coding for an IgE variable region
gtcctgtcccaggtgcagctgctcgagtcgggcccaggactggtgaagccttcagagaccgtgtccctcacctgcactgtctctggggactccatcaacagtgcctacaactactggagctggatccgacagcccgccgggaagggactggagtggattgggcgtatgtacaccaatgggggcaccggtgggaacaccttctaccacccctccctcaagagtcgactcaccatgtcagtggacacgtccaagaaccatttctccctgaagctcaactctgtgaccgccgcggacacggccatatattactgtgcgcgagatcggggagccatcgaccattactactggggccggggaaccctggtcaccgtctcgtcag

SEQ ID NO: 121 - DNA, 379 bp, Artificial
  DNA sequence coding for an IgE variable region
gtcctgtcccaggtgcagctgctcgagtcgggcccaggactggtgaagccttcagagaccgtgtccctcacctgcactgtctctggggactccatcaacagtgcctacaactactggagctggatccgacagcccgccgggaagggactggagtggattgggcgtatgtacaccaatgggggcaccggtgggaacaccttctaccaccccgccctcaagagtcgactcaccatgtcagtggacacgtccaagaaccatttctccctgaagctcaactctgtgaccgccgcggacacggccatatattactgtgcgcgagatcggggagccatcgaccattactactggggccggggaaccctggtcaccgtctcgtcag

SEQ ID NO: 122 - DNA, 379 bp, Artificial; identical to SEQ ID NO: 117
  DNA sequence coding for an IgE variable region

SEQ ID NO: 123 - DNA, 382 bp, Artificial
  DNA sequence coding for an IgE variable region
gtcctgtcccaggtgcagctgctcgagtcggggggagggttggtacagcctggggggtccctgagactctcctgtgcagcctctaaattcacctttagcagccatgccatgacctgggtccgccaggctccagggaaggggctggaatgggtctcaagtattagtaatggtggcggtactacttactacacagactccgtgaagggccggttcaccatctccagagacaattccaaggacatgctgtatctgcaaatgaacaccctgagagtcgaggacacggccgtttatttctgtggaagatatggttcggggaattatactcacttctactacggcctggacgtctggggccatgggaccacggtcaccgtctcctcag

SEQ ID NO: 124 - DNA, 379 bp, Artificial; identical to SEQ ID NO: 113
  DNA sequence coding for an IgE variable region

SEQ ID NO: 125 - DNA, 388 bp, Artificial
  DNA sequence coding for an IgE variable region
gtcctgtcccaggtgcagctgctcgagtcgggcccaggactggtgaagccttcacagaccctgtccctcacctgcactgtcgctggttactctatcagcagtggtcgttattactggacttggatccggcagtccgccagtaagggactggagtggattggccgtatccatatcagtggggacacaaattacaatccctccttcaagagtcgagtcaccatgtcagaggacatgtccaagaaccagttctccctgaagctgaggtctgtgaccgccgcagacacggccgtgtatttttgtgcgcgagtccgggacgacccagagcgggccacaattcagtatgcttttgatatctggggccaagggacaatggttaccgtgtcctcag

SEQ ID NO: 126 - DNA, 376 bp, Artificial
  DNA sequence coding for an IgE variable region
gtcctgtcccaggtgcagctgctcgagtcgggcccaggactggtgaagccttcggagactctagacctcacgtgcaatgtgtctggtgctcctatcgacaggagtattgtcctctgggcctggatccgccagaccccaggcaaggccctggagtggattgcgtctgtctcttttagtgggaacacctatcacaatccctccctaaagagtcgagtcaccgtgcccgtcgacacgtccaggaaccagttctccctgaggctcagctctgtgaccgccgccgacactggaatttattactgtgcgagacggggaattattgcgggtgtcagtcactttgactactggggccagggagccctggtcttcgtctcctccg

SEQ ID NO: 127 - DNA, 384 bp, Artificial
  DNA sequence coding for an IgE variable region
gtcctgtcccaggtgcagctgctcgagtcgggcccaggactggtgaagccttcacagaccctgtccgtcacatgcgctgtctctggtgcctctgtctctagaggtggttattactggacctggctccgccagccgccaggaagggcctggagtggattggcgacatttattacactggggccacctactacaacccgtcactcaagcccagatttacgatgtcaataaacgcgtctaacaaccagttctccctgaaggtggcctctgtgactgtcgccgacacggccacttattattgtgcgagaatcccggcggcacacacgtcctccggcccacaaggcttcgacccctggggccagggaaccctggtcaccgtctcctcag

SEQ ID NO: 128 - DNA, 391 bp, Artificial; identical to SEQ ID NO: 116
  DNA sequence coding for an IgE variable region

SEQ ID NO: 129 - DNA, 317 bp, Artificial
  DNA sequence coding for an IgE variable region
gtctgtgccgaggtgcagctgctcgagtctgggggaggctgggtaaagcctggggggtcccttagactctcctgtgcagcctctggattcactttcagtgatggtgggacaacagacttcggtgcacccgtgaaaggcagattcaccgtctcaagagatgactcaaaagacacactgtatttgtcaatgaacagcctgaaaacagaggacacagccgtctatgactgttctggtcctttgagagtagtggttattactcaaacagcgatactactctgactgccggggccagggaaccctggtcaccgtctcttcag

SEQ ID NO: 130 - DNA, 368 bp, Artificial
  DNA sequence coding for an IgE variable region
gtctgtgccgaggtgcagctgctcgagtctgggcccaggactggcgaagccttcacagaccctgtccctcacctgcactgtctctggtggctccatcaccagtggtgattactactggaattggatccgccagtccccagggaagggcctggagtggattggatacctcttttatagtgggggcacctactacagcccgaccctcaagagtcgagttaccatatcagttgacacgtccaagaaccagttctccctgaacgtgacctctctgactgccgcagacacggccgtctattactgtgccagagggggctgggaggatgcctttgatatctggggtcaagggactttggtcaccgtctcttcag

SEQ ID NO: 131 - DNA, 364 bp, Artificial
  DNA sequence coding for an IgE variable region
gtctgtgccgaggtgcagctgctcgagtctggggctgaggggaagaagcctggggcctcagtgaacgtctcctgtcagacttctggatacaccttctccgcctactatatacactgggtgcgacaggcccctggacaagggcttgagtggatggggtacatcaaccctcacactggtggcacacactctgcacagaagtttcagggctgggtcaccatgaccagggacacgtccatcagcacagcctacatggagatgaagagtctgaaatctgacgacgcggccgtttattactgtgcgagagggggccgcggctggttccttgatcactggggccagggaaccctggtcgccgtgtcgtcag

SEQ ID NO: 132 - DNA, 379 bp, Artificial
  DNA sequence coding for an IgE variable region
gtcctgtcacaggtacagctgctagagtcaggcccnggactggtgaagccttcagagaccgtgtccctcacctgcactgtctctggggactccatcaacagtgcctacaactgctggagctggatccgacagcccgccgggaagggactggagtggattgggcgtatgtacaccaatgggggcaccggtgggaacaccttctaccacccctccctcaagagtcgactcaccatgtcagtggacacgtccaggaaccatttctccctgaagctcaactctgtgaccgccgcggacacggccatatattactgtgcgcgagatcggggagccatcgaccattactactggggccggggaaccctggtcaccgtctcgtcag

SEQ ID NO: 133 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 134 - DNA, 341 bp, Artificial; identical to SEQ ID NO: 133
  DNA sequence coding for an IgE variable region

SEQ ID NO: 135 - DNA, 341 bp, Artificial; identical to SEQ ID NO: 133
  DNA sequence coding for an IgE variable region

SEQ ID NO: 136 - DNA, 341 bp, Artificial; identical to SEQ ID NO: 133
  DNA sequence coding for an IgE variable region

SEQ ID NO: 137 - DNA, 341 bp, Artificial; identical to SEQ ID NO: 133
  DNA sequence coding for an IgE variable region

SEQ ID NO: 138 - DNA, 341 bp, Artificial; identical to SEQ ID NO: 133
  DNA sequence coding for an IgE variable region

SEQ ID NO: 139 - DNA, 341 bp, Artificial; identical to SEQ ID NO: 133
  DNA sequence coding for an IgE variable region

SEQ ID NO: 140 - DNA, 341 bp, Artificial; identical to SEQ ID NO: 133
  DNA sequence coding for an IgE variable region

SEQ ID NO: 141 - DNA, 341 bp, Artificial; identical to SEQ ID NO: 133
  DNA sequence coding for an IgE variable region

SEQ ID NO: 142 - DNA, 341 bp, Artificial; identical to SEQ ID NO: 133
  DNA sequence coding for an IgE variable region

SEQ ID NO: 143 - DNA, 341 bp, Artificial; identical to SEQ ID NO: 133
  DNA sequence coding for an IgE variable region

SEQ ID NO: 144 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttaaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 145 - DNA, 341 bp, Artificial; identical to SEQ ID NO: 133
  DNA sequence coding for an IgE variable region

SEQ ID NO: 146 - DNA, 341 bp, Artificial; identical to SEQ ID NO: 133
  DNA sequence coding for an IgE variable region

SEQ ID NO: 147 - DNA, 341 bp, Artificial; identical to SEQ ID NO: 133
  DNA sequence coding for an IgE variable region

SEQ ID NO: 148 - DNA, 341 bp, Artificial; identical to SEQ ID NO: 133
  DNA sequence coding for an IgE variable region

SEQ ID NO: 149 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtgggtctcaaagtattagtagtagtggtggtagcacatctacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 150 - DNA, 359 bp, Artificial
  DNA sequence coding for an IgE variable region
gggaggcttggtcaagccgggcgggtcccttagactctcctgtgcagcctctggtctcactttcagtgacgcctggatgaactgggtccgccaggctccagggaaggggctggagtgggtcggccgtattcaaagtaaatctgatggtgagacaacagcctacgctccactcgtgacaggcagattctccatttcaagagatgattcaaaaaacatgttgtatctgcatatgaacaggctgaaaacggaggacacagccgtatattattgttgcacaggtgaaaggggcgattctcggagtggctattgggggtcgtttgactactggggccagggaaacctggtcaccgtctcttcag

SEQ ID NO: 151 - DNA, 341 bp, Artificial; identical to SEQ ID NO: 149
  DNA sequence coding for an IgE variable region

SEQ ID NO: 152 - DNA, 341 bp, Artificial; identical to SEQ ID NO: 133
  DNA sequence coding for an IgE variable region

SEQ ID NO: 153 - DNA, 341 bp, Artificial; identical to SEQ ID NO: 133
  DNA sequence coding for an IgE variable region

SEQ ID NO: 154 - DNA, 341 bp, Artificial; identical to SEQ ID NO: 133
  DNA sequence coding for an IgE variable region

SEQ ID NO: 155 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagggacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 156 - DNA, 341 bp, Artificial; identical to SEQ ID NO: 133
  DNA sequence coding for an IgE variable region

SEQ ID NO: 157 - DNA, 341 bp, Artificial; identical to SEQ ID NO: 133
  DNA sequence coding for an IgE variable region

SEQ ID NO: 158 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctccttag

SEQ ID NO: 159 - DNA, 341 bp, Artificial; identical to SEQ ID NO: 133
  DNA sequence coding for an IgE variable region

SEQ ID NO: 160 - DNA, 341 bp, Artificial; identical to SEQ ID NO: 133
  DNA sequence coding for an IgE variable region

SEQ ID NO: 161 - DNA, 341 bp, Artificial; identical to SEQ ID NO: 133
  DNA sequence coding for an IgE variable region

SEQ ID NO: 162 - DNA, 341 bp, Artificial; identical to SEQ ID NO: 133
  DNA sequence coding for an IgE variable region

SEQ ID NO: 163 - DNA, 341 bp, Artificial; identical to SEQ ID NO: 133
  DNA sequence coding for an IgE variable region

SEQ ID NO: 164 - DNA, 338 bp, Artificial
  DNA sequence coding for an IgE variable region
gggaagcttggtacagcctggggggtccctgagactctcctgtgcagcctctggattcagcttcagtaactatgccatgagctgggtccgccaggctccagggaaggggcttgagttgggtctcaactattactggtagtggtggagcacgtactacgcagactccgtgcagggccgcttcaccatctccagagacaattccaacaacacactgtatctgcaaatgaacggcctgacagccgaggacacggccatatattactgtgcgaaaaggccaggccgggggagggtcccggacgacccctggggccagggaaccctggtcattgtctcctcag

SEQ ID NO: 165 - DNA, 338 bp, Artificial
  DNA sequence coding for an IgE variable region
aggaggcttgatccagcctggggggtccctgagactctcctgtgcagcctccggcttcagcgtcagtagcaactacatgagctgggtccgccaggctccagggaaggggctggagtgggtctcagtaatctatcgcggtggtagcacatactacgcagactccgtgaagggccgattcaccatctccagagacaattccaagaacacgctgcatcttcaaatgaacaccctgagagccgaggacacggccgtgtactactgtgcgagagccagtgggagttatttcatctggtacttcgctctctggggccgtggcaccctggtcactgtctcctcag

SEQ ID NO: 166 - DNA, 341 bp, Artificial
  DNA sequence coding for an IgE variable region
aggaggcttgattcagcctggggggtccctgagactctcctgtgcagcctctgggttcaccgtcagtaggaactacatgacctgggtccgccaggcccccagggaaggggctggagtgggtctcagtatttatagaggcggcaccgcatactacgcagactccgtgaagggccgattcaccatctccagagacaattccaccaacaccctgtatcttcaaatgagcagtctgagagccgaggacacggccgtctattactgtgcgagagtagaggtgggttactactattactacgggatggacgtctggggccaagggaccacggtcaccgtctcctcag

SEQ ID NO: 167 - DNA, 365 bp, Artificial
  DNA sequence coding for an IgE variable region
cccaggactggtgaagccttcggggaccctgtccctcacctgcactgtctctggtggctccatcagcagtagtacctggtggacttgggtccgccagcccccagagaaggggctggagtggattggggaaatctattatagtggagacaccaactacaacccgtccctcaagagtcgagtcaccatatccacagacaagtccaagaaccacttctccctgaatctgacctctgtgaccgccgcggacacggccgtgtattactgtgcgaggacgaaggggggcttgaaaggctacgatttttggagtggttacccgtactactttgactgctggggccagggagtcctggtccccgtctcctcag

SEQ ID NO: 168 - DNA, 335 bp, Artificial
  DNA sequence coding for an IgE variable region
cccaggactggtgaagccttcggagaccctgtccctcacctgcactgtctctggtggctccgtcagcagtgagagtgcctactggagttggatccgacagcccccagggaagggactggagtggattggatatattcatcacagtgggaagaccaactacaacccctcactcaagagtcgagtcaccatatcagtcgacacgtccaagaaccaattctccctgaacctgaggtctgtgaccgccgcggacacggctgtatatttctgtgcgagagatttggctatttccttctttgactgctggggccagggaaccctggtcaccgtctcttcag

SEQ ID NO: 169 - DNA, 341 bp, Artificial; identical to SEQ ID NO: 133
  DNA sequence coding for an IgE variable region

SEQ ID NO: 170 - DNA, 341 bp, Artificial; identical to SEQ ID NO: 133
  DNA sequence coding for an IgE variable region

SEQ ID NO: 171 - DNA, 341 bp, Artificial; identical to SEQ ID NO: 133
  DNA sequence coding for an IgE variable region

SEQ ID NO: 172 - DNA, 341 bp, Artificial; identical to SEQ ID NO: 133
  DNA sequence coding for an IgE variable region

SEQ ID NO: 173 - DNA, 341 bp, Artificial; identical to SEQ ID NO: 133
  DNA sequence coding for an IgE variable region

SEQ ID NO: 174 - DNA, 359 bp, Artificial; identical to SEQ ID NO: 150
  DNA sequence coding for an IgE variable region

SEQ ID NO: 175 - DNA, 341 bp, Artificial
  DNA sequence coding for an IgE variable region
gggagacttggtacagcctggggggtcccttagagtctcctgtgcagcctccggattcacgtttagcagctatgccatggcctgggtctgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 176 - DNA, 341 bp, Artificial; identical to SEQ ID NO: 133
  DNA sequence coding for an IgE variable region

SEQ ID NO: 177 - DNA, 341 bp, Artificial; identical to SEQ ID NO: 133
  DNA sequence coding for an IgE variable region

SEQ ID NO: 178 - DNA, 341 bp, Artificial; identical to SEQ ID NO: 133
  DNA sequence coding for an IgE variable region

SEQ ID NO: 179 - DNA, 341 bp, Artificial; identical to SEQ ID NO: 133
  DNA sequence coding for an IgE variable region

SEQ ID NO: 180 - DNA, 341 bp, Artificial; identical to SEQ ID NO: 133
  DNA sequence coding for an IgE variable region

SEQ ID NO: 181 - DNA, 341 bp, Artificial; identical to SEQ ID NO: 133
  DNA sequence coding for an IgE variable region

SEQ ID NO: 182 - DNA, 341 bp, Artificial; identical to SEQ ID NO: 133
  DNA sequence coding for an IgE variable region

SEQ ID NO: 183 - DNA, 341 bp, Artificial; identical to SEQ ID NO: 133
  DNA sequence coding for an IgE variable region

SEQ ID NO: 184 - DNA, 341 bp, Artificial; identical to SEQ ID NO: 133
  DNA sequence coding for an IgE variable region

SEQ ID NO: 185 - DNA, 341 bp, Artificial; identical to SEQ ID NO: 133
  DNA sequence coding for an IgE variable region

SEQ ID NO: 186 - DNA, 341 bp, Artificial; identical to SEQ ID NO: 133
  DNA sequence coding for an IgE variable region

SEQ ID NO: 187 - DNA, 341 bp, Artificial; identical to SEQ ID NO: 133
  DNA sequence coding for an IgE variable region

SEQ ID NO: 188 - DNA, 341 bp, Artificial; identical to SEQ ID NO: 133
  DNA sequence coding for an IgE variable region

SEQ ID NO: 189 - DNA, 341 bp, Artificial; identical to SEQ ID NO: 133
  DNA sequence coding for an IgE variable region

SEQ ID NO: 190 - DNA, 341 bp, Artificial; identical to SEQ ID NO: 133
  DNA sequence coding for an IgE variable region

SEQ ID NO: 191 - DNA, 341 bp, Artificial; identical to SEQ ID NO: 133
  DNA sequence coding for an IgE variable region

SEQ ID NO: 192 - DNA, 341 bp, Artificial; identical to SEQ ID NO: 133
  DNA sequence coding for an IgE variable region

SEQ ID NO: 193 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctgaagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 194 - DNA, 341 bp, Artificial; identical to SEQ ID NO: 133
  DNA sequence coding for an IgE variable region

SEQ ID NO: 195 - DNA, 341 bp, Artificial; identical to SEQ ID NO: 133
  DNA sequence coding for an IgE variable region

SEQ ID NO: 196 - DNA, 341 bp, Artificial; identical to SEQ ID NO: 133
  DNA sequence coding for an IgE variable region

SEQ ID NO: 197 - DNA, 341 bp, Artificial; identical to SEQ ID NO: 133
  DNA sequence coding for an IgE variable region

SEQ ID NO: 198 - DNA, 341 bp, Artificial; identical to SEQ ID NO: 133
  DNA sequence coding for an IgE variable region

SEQ ID NO: 199 - DNA, 341 bp, Artificial; identical to SEQ ID NO: 133
  DNA sequence coding for an IgE variable region

SEQ ID NO: 200 - DNA, 341 bp, Artificial; identical to SEQ ID NO: 133
  DNA sequence coding for an IgE variable region

SEQ ID NO: 201 - DNA, 341 bp, Artificial; identical to SEQ ID NO: 133
  DNA sequence coding for an IgE variable region

SEQ ID NO: 202 - DNA, 341 bp, Artificial; identical to SEQ ID NO: 133
  DNA sequence coding for an IgE variable region

SEQ ID NO: 203 - DNA, 341 bp, Artificial; identical to SEQ ID NO: 133
  DNA sequence coding for an IgE variable region

SEQ ID NO: 204 - DNA, 341 bp, Artificial; identical to SEQ ID NO: 133
  DNA sequence coding for an IgE variable region

SEQ ID NO: 205 - DNA, 341 bp, Artificial; identical to SEQ ID NO: 133
  DNA sequence coding for an IgE variable region

SEQ ID NO: 206 - DNA, 341 bp, Artificial; identical to SEQ ID NO: 133
  DNA sequence coding for an IgE variable region

SEQ ID NO: 207 - DNA, 341 bp, Artificial
  DNA sequence coding for an IgE variable region
ggnagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 208 - DNA, 342 bp, Artificial
  DNA sequence coding for an IgE variable region
gangagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 209 - DNA, 341 bp, Artificial; identical to SEQ ID NO: 133
  DNA sequence coding for an IgE variable region

SEQ ID NO: 210 - DNA, 341 bp, Artificial; identical to SEQ ID NO: 133
  DNA sequence coding for an IgE variable region

SEQ ID NO: 211 - DNA, 341 bp, Artificial; identical to SEQ ID NO: 133
  DNA sequence coding for an IgE variable region

SEQ ID NO: 212 - DNA, 341 bp, Artificial; identical to SEQ ID NO: 133
  DNA sequence coding for an IgE variable region

SEQ ID NO: 213 - DNA, 341 bp, Artificial; identical to SEQ ID NO: 133
  DNA sequence coding for an IgE variable region

SEQ ID NO: 214 - DNA, 341 bp, Artificial; identical to SEQ ID NO: 133
  DNA sequence coding for an IgE variable region

SEQ ID NO: 215 - DNA, 341 bp, Artificial; identical to SEQ ID NO: 133
  DNA sequence coding for an IgE variable region

SEQ ID NO: 216 - DNA, 341 bp, Artificial; identical to SEQ ID NO: 133
  DNA sequence coding for an IgE variable region

SEQ ID NO: 217 - DNA, 341 bp, Artificial; identical to SEQ ID NO: 133
  DNA sequence coding for an IgE variable region

SEQ ID NO: 218 - DNA, 341 bp, Artificial; identical to SEQ ID NO: 133
  DNA sequence coding for an IgE variable region

SEQ ID NO: 219 - DNA, 341 bp, Artificial; identical to SEQ ID NO: 133
  DNA sequence coding for an IgE variable region

SEQ ID NO: 220 - DNA, 341 bp, Artificial; identical to SEQ ID NO: 133
  DNA sequence coding for an IgE variable region

SEQ ID NO: 221 - DNA, 359 bp, Artificial; identical to SEQ ID NO: 150
  DNA sequence coding for an IgE variable region

SEQ ID NO: 222 - DNA, 341 bp, Artificial; identical to SEQ ID NO: 133
  DNA sequence coding for an IgE variable region

SEQ ID NO: 223 - DNA, 341 bp, Artificial; identical to SEQ ID NO: 133
  DNA sequence coding for an IgE variable region

SEQ ID NO: 224 - DNA, 341 bp, Artificial; identical to SEQ ID NO: 133
  DNA sequence coding for an IgE variable region

SEQ ID NO: 225 - DNA, 341 bp, Artificial
  DNA sequence coding for an IgE variable region
gggagacttggtacagcctggggggtcccttagagtctcctgtgtagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 226 - DNA, 341 bp, Artificial; identical to SEQ ID NO: 133
  DNA sequence coding for an IgE variable region

SEQ ID NO: 227 - DNA, 341 bp, Artificial; identical to SEQ ID NO: 133
  DNA sequence coding for an IgE variable region

SEQ ID NO: 228 - DNA, 341 bp, Artificial; identical to SEQ ID NO: 133
  DNA sequence coding for an IgE variable region

SEQ ID NO: 229 - DNA, 341 bp, Artificial; identical to SEQ ID NO: 133
  DNA sequence coding for an IgE variable region

SEQ ID NO: 230 - DNA, 341 bp, Artificial; identical to SEQ ID NO: 133
  DNA sequence coding for an IgE variable region

SEQ ID NO: 231 - DNA, 341 bp, Artificial; identical to SEQ ID NO: 133
  DNA sequence coding for an IgE variable region

SEQ ID NO: 232 - DNA, 341 bp, Artificial; identical to SEQ ID NO: 133
  DNA sequence coding for an IgE variable region

SEQ ID NO: 233 - DNA, 341 bp, Artificial; identical to SEQ ID NO: 133
  DNA sequence coding for an IgE variable region

SEQ ID NO: 234 - DNA, 341 bp, Artificial
  DNA sequence coding for an IgE variable region
ggggagacttggtacagcctggggggtcccttagagtctcctgtgcagcctccggattcacgtttagcagctatgccatgacctgggtccgccaggctccagggaaggggctggagtgggtctcaagtattgtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 235 - DNA, 290 bp, Artificial
  DNA sequence coding for an IgE variable region
cggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattacctgtgcgaaaggagatcgatttttggagtgcccacacctgacttttggggccagggaaccctggtcaccgtctcctcag

SEQ ID NO: 236 - DNA, 300 bp, Artificial
  DNA sequence coding for an IgE variable region
gtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggcccgtatattactgtgcgaaaggagatcgattttggagtggcccacacctgacttttggggccagggaaccctggtcaccgtctcctcag

SEQ ID NO: 237 - DNA, 319 bp, Artificial
  DNA sequence coding for an IgE variable region
gggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagccctgagagtcggggacacggccgtatattatgtgcgaaaggagatcgatttttggagtggcccacacctgacttttggggccagggaaccctggtcaccgtctcctcag

SEQ ID NO: 238 - DNA, 319 bp, Artificial; identical to SEQ ID NO: 237
  DNA sequence coding for an IgE variable region

SEQ ID NO: 239 - DNA, 304 bp, Artificial
  DNA sequence coding for an IgE variable region
tcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacaacggccgtatattactgtgcgaaaggagacgatttttggagtggcccacacctgacttttggggccagggaaccctggtcaccgtctcctcag

SEQ ID NO: 240 - DNA, 341 bp, Artificial; identical to SEQ ID NO: 133
  DNA sequence coding for an IgE variable region

SEQ ID NO: 241 - DNA, 341 bp, Artificial; identical to SEQ ID NO: 133
  DNA sequence coding for an IgE variable region

SEQ ID NO: 242 - DNA, 341 bp, Artificial; identical to SEQ ID NO: 133
  DNA sequence coding for an IgE variable region

SEQ ID NO: 243 - DNA, 341 bp, Artificial; identical to SEQ ID NO: 133
  DNA sequence coding for an IgE variable region

SEQ ID NO: 244 - DNA, 341 bp, Artificial; identical to SEQ ID NO: 133
  DNA sequence coding for an IgE variable region

SEQ ID NO: 245 - DNA, 341 bp, Artificial; identical to SEQ ID NO: 133
  DNA sequence coding for an IgE variable region

SEQ ID NO: 246 - DNA, 341 bp, Artificial; identical to SEQ ID NO: 133
  DNA sequence coding for an IgE variable region

SEQ ID NO: 247 - DNA, 341 bp, Artificial; identical to SEQ ID NO: 133
  DNA sequence coding for an IgE variable region

SEQ ID NO: 248 - DNA, 341 bp, Artificial; identical to SEQ ID NO: 133
  DNA sequence coding for an IgE variable region

SEQ ID NO: 249 - DNA, 341 bp, Artificial; identical to SEQ ID NO: 133
  DNA sequence coding for an IgE variable region

SEQ ID NO: 250 - DNA, 341 bp, Artificial; identical to SEQ ID NO: 133
  DNA sequence coding for an IgE variable region

SEQ ID NO: 251 - DNA, 341 bp, Artificial; identical to SEQ ID NO: 133
  DNA sequence coding for an IgE variable region

SEQ ID NO: 252 - DNA, 341 bp, Artificial; identical to SEQ ID NO: 133
  DNA sequence coding for an IgE variable region

SEQ ID NO: 253 - DNA, 341 bp, Artificial; identical to SEQ ID NO: 133
  DNA sequence coding for an IgE variable region

SEQ ID NO: 254 - DNA, 341 bp, Artificial; identical to SEQ ID NO: 133
  DNA sequence coding for an IgE variable region

SEQ ID NO: 255 - DNA, 341 bp, Artificial; identical to SEQ ID NO: 133
  DNA sequence coding for an IgE variable region

SEQ ID NO: 256 - DNA, 341 bp, Artificial; identical to SEQ ID NO: 133
  DNA sequence coding for an IgE variable region

SEQ ID NO: 257 - DNA, 341 bp, Artificial; identical to SEQ ID NO: 133
  DNA sequence coding for an IgE variable region

SEQ ID NO: 258 - DNA, 341 bp, Artificial; identical to SEQ ID NO: 133
  DNA sequence coding for an IgE variable region

SEQ ID NO: 259 - DNA, 341 bp, Artificial; identical to SEQ ID NO: 133
  DNA sequence coding for an IgE variable region

SEQ ID NO: 260 - DNA, 341 bp, Artificial; identical to SEQ ID NO: 133
  DNA sequence coding for an IgE variable region

SEQ ID NO: 261 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtggggtctcaagtattagtagtagtggtggtacacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 262 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggaggtgggtctcaagtattagtagtagtggtgtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 263 - DNA, 341 bp, Artificial; identical to SEQ ID NO: 261
  DNA sequence coding for an IgE variable region

SEQ ID NO: 264 - DNA, 341 bp, Artificial
  DNA sequence coding for an IgE variable region
gggagacttggtacagcctggggggtcccttagagtctcctgtgcagcctccggattcacgtttagcagctatgccatgacctgggtccgccaggctccagggaaggggctggagtggggtctcaagtattagtagtagtggtggtagacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 265 - DNA, 366 bp, Artificial
  DNA sequence coding for an IgE variable region
gcccaggactggtgaagccttcggagaccctgtccctcacctgcactgtctctggtggctccatcagcagtagtacctggtggacttgggtccgccagcccccagggaaggggctggaatggattggggaaatctattatagtggggacaccaactacaagccgtccctcaagagtcgagtcaccatatcaattgacaagtccaagaaccagttctccctgaacctgagctctgtgaccgccgcggacacggccgtctattactgtgcgagaacgaaggggggcttgaaaggctacgatttttggagtggttacccatattactttgactactggggccagggaaccctggtcaccgtctcctcag

SEQ ID NO: 266 - DNA, 350 bp, Artificial
  DNA sequence coding for an IgE variable region
cccaggactggtgaagccttcgcagaccctgtccctcacctgcactgtctctggtggctccatcggcagtaatactgacttctgggactggatccgccagcccccagggaagggactggagtggattgggagtatcgattacagtgggttcacctacttcaatccgtccctcaagagtcgaatcaccatctccgaagacacgtccaagaaccagttctccctgaaactgacctctgtgaccgccgcagacacggctgtctattactgtgcgagacatcgggacggtggtaaagtctgggagtactactttgacttctggggccagggaaccctggtcaccgtctcctcag

SEQ ID NO: 267 - DNA, 362 bp, Artificial
  DNA sequence coding for an IgE variable region
cccaggactggtgaagccttcacagaccctgtccctcacctgcactgtctctgctggctccatcagcggtagtggttactactggacctggatccgccagcacccagggaagggcctggagtggattggctacatctattacagtgggagcacctactacaacccgtcgctcaagagtcgagttaccatatcagcagatacgtctaagaaccagttctccctgaggttgagctctgtgactgccgcggacacggccgtgtattactgtgcgagaggcccgtattactatggttcagggagtcatcggtactactactacatggacgtctggggcaaagggaccacggtcaccgtctcctcag

SEQ ID NO: 268 - DNA, 362 bp, Artificial; identical to SEQ ID NO: 267
  DNA sequence coding for an IgE variable region

SEQ ID NO: 269 - DNA, 363 bp, Artificial
  DNA sequence coding for an IgE variable region
actcccaggtgcagctgctcgagtctggggctgaggtgaagaggcctggggcctcagtgaaagtctcctgtaagacttctggatacaccttccccctctcctatatccactgggtgcgacaggcccctggacaagggcttgagtacatgggacgggtcaacccttacaatggtggctcacactcctcacagaagtttcagggcagggtcaacatgaccagggacacgtccatcaccacagcctacatggaattgaatagcctgacatctgacgacacggccgtatattattgtacgagggaagtggctggtaggagagagtttttggactggggccagggagccctggtcaccgtctcctcag

SEQ ID NO: 270 - DNA, 260 bp, Artificial; identical to SEQ ID NO: 37
  DNA sequence coding for an IgE variable region

SEQ ID NO: 271 - DNA, 369 bp, Artificial
  DNA sequence coding for an IgE variable region
actcccaggtgcagctgctcgagtctgggggagacttggtacagcctggggggtcccttagagtctcctgtgcagcctccggattcacgtttagcagctatgccatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 272 - DNA, 368 bp, Artificial
  DNA sequence coding for an IgE variable region
actcccaggtgcagctgctcgagtctgggggagacttggtacagcctggggggtcccttagagtctcctgtgcagcctccggattcacgtttagcagctatgcatgacctgggtccgccaggctccagggaaggggctggagtgggtctcaagtattagtagtagtggtggtagcacatactacgcagactccgtgaagggccggttcaccatctccagagacaattccaagaacacgttgtttctgcaaatgagcagcctgagagtcggggacacggccgtatattactgtgcgaaaggagatcgatttttggagtggcccacacctgacttttggggccagggaaccctggtcaccgtctcctcag

SEQ ID NO: 273 - DNA, 369 bp, Artificial
  DNA sequence coding for an IgE variable region
actcccaggtgcagctgctcgagtctgggggaggctcggtacagcctggggggtccctgagactctcctgtgtaggctttggattcacatgtagcatctttcccatgagctgggtccgccaggctccagggaaggggctggagtgggtctcagccattagcggtactgagagcagtacgtattattcggactccgtgaagggccggttcaccatctccagagactgttccaagaacacagtgtatctgcatatgaacagcctgagcggcgaggactcggccgtctattactgtgcgaaaccagcaggtatcacagcagctggctactttgaattttggggccagggaaccctggtcgccgtctcttcgg

SEQ ID NO: 274 - DNA, 369 bp, Artificial
  DNA sequence coding for an IgE variable region
actcccaggtgcagctgctcgagtctgggggaggctcggtacagcctggggggtccctgagactctcctgtgtaggctttggattcacatgtagcatctttcccatgagctgggtccgccaggctccagggaaggggctggagtgggtctcagccattagcggtactgagagcagtacatattattcggactccgtgaagggccggttcaccatctccagagactgttccaagaacacagtgtatctgcatatgaacagcctgagcggcgaggactcggccgtctattactgtgcgaaaccagcaggtatcacagcagctggctactttgaattttggggccagggaaccctggtcgccgtctcttcgg

SEQ ID NO: 275 - DNA, 387 bp, Artificial
  DNA sequence coding for an IgE variable region
actcccaggtgcagctgctcgagtctgggggaggcttggtcaagccgggcgggtcccttagactctcctgtgcagcctctggtctcactttcagtgacgcctggatgaactgggtccgccaggctccagggaaggggctggagtgggtcggccgtattcaaagtaaatctgatggtgagacaacagcctacgctccactcgtgacaggcagattctccatttcaagagatgattcaaaaaacatgttgtatctgcatatgaacaggctgaaaacggaggacacagccgtatattattgttgcacaggtgaaaggggcgattctcggagtggctattgggggtcgtttgactactggggccagggaaacctggtcaccgtctcttcag

SEQ ID NO: 276 - DNA, 372 bp, Artificial
  DNA sequence coding for an IgE variable region
actcccaggtgcagctgctcgagtctgggggaggcgtggtcctgcctgggaggtccctgagactctcatgtgtggcatctggattcaccctcagtaatcatggccatgcactgggtccgccaggctccaggcaggggctggagtggctggcagttatctggcataatggaagtaacaaatactatggagagtccgtaaagggccgactcatcatttccagagacaattccaagaacacggtgtacctgcaaatcaacagcctgagagccggggacacggctgtatattcgtgtgcgagagattttgaccggggaggcttttactactacatggacttgtggggcagagggaccacggtcaccgtctcctcag

SEQ ID NO: 277 - DNA, 382 bp, Artificial
  DNA sequence coding for an IgE variable region
gtcctgtcccaggtgcagctgctcgagtcgggcccaggactggtgaagccttcgcagaccctgtccctcacctgcactgtctctggtggctccatcggcagtaatactgacttctgggactggatccgccagcccccagggaagggactggagtggattgggagtatcgattacagtgggttcacctacttcaatccgtccctcaagagtcgaatcaccatctccgaagacacgtccaagaaccagttctccctgaaactgacctctgtgaccgccgcagacacggctgtctattactgtgcgagacatcgggacggtggtaaagtctgggagtactactttgacttctggggccagggaaccctggtcaccgtctcctcag

SEQ ID NO: 278 - DNA, 377 bp, Artificial
  DNA sequence coding for an IgE variable region
gtcccaggtgcagctgctcgagtcgggcccaggactggtgaagccttcgcagaccctgtccctcacctgcactgtctctggtggctccatcggcagtaatactgacttctgggactggatccgccagcccccagggaagggactggagtggattgggagtatcgattacagtgggcgcacctacttcaatccgtccctcaagagtcgaatcaccatatccgtagacacgtccaagacccagttctccctgaaactgacctctgtgaccgccgcagacacggctgtctattactgtgcgagacatcgggacggtggtaaagtctgggagtactactttgacttctggggccagggaaccctggtcaccgtctcctcag

SEQ ID NO: 279 - DNA, 391 bp, Artificial
  DNA sequence coding for an IgE variable region
ctgtcccaggtgcagctgctcgagtcgggcccaggactggtgaagccttcacagaccctgtccctcacctgcactgtctctgctggctccatcagcggtggtggttactactggacctggatccgccagcacccagggaagggcctggagtggattggctacatctattacagtgggagcgcctactacaacccgtccctcaagagtcgagttaccatatcagtagacacgtctcagaaccagttctccctgaggttgagctctgtgactgccgcggacacggccgtttattactgtgcgagaggcccgtattactacggttcagggagtcatcgctactactactacatggacgtctggggcaaagggaccacggtcaccgtctcctcag

SEQ ID NO: 280 - DNA, 338 bp, Artificial
  DNA sequence coding for an IgE variable region
cccaggactggtgaagccttcggagaccctgtctctcaactgcactgtctctggtgcttccgtcagcagtggcagttatcactggagttggatccggcagcccccagggaagggactggagtgggttgcatatatctattccagggggggcaccaattacaacccctccctcaagagtcgagtcaccatatcagtggacacgtccaagaaccagttgtccctgaagctgagctctgtgaccgctgcggacacggccatatattactgtgtgagaacaggaatgataataagatacatggacgtctggggcaaagggaccacggtcaccgtctcctcag

SEQ ID NO: 281 - DNA, 351 bp, Artificial
  DNA sequence coding for an IgE variable region
actcccaggtgaagctgctcgagtctgggactgaggtgaagaagcctggggcctcagtgaaggtctcctgcaaggcttctggatacgtcttcaccgcctattatctgcactggatgcgacaggcccctggacaagggcctgagtggatgggacgcataagccccagtagtggggacacaatctatgcacagaactttcaaggcagggccaccatgaccagagacacgtccatcagcacagcctacctggagctgaacagactgacgtctgccgacacggccgtctattactgtgcgagagataatattatcgacccctggggccagggaaccttggtcaccgtctcctcag

SEQ ID NO: 282 - DNA, 351 bp, Artificial
  DNA sequence coding for an IgE variable region
actcccaggtgcagctgctcgagtctgggactgaggtgaagaagcctggggcctcagtgaaggtctcctgcaaggcttctggatacgtcttcaccgcctattatctgcactggatgcgacaggcccctggacaagggcctgagtggatgggacgcataagccccagtagtggggacacaatctatgcacagaactttcaaggcagggccaccatgaccagagacgcgtccatcagcacagcctacctggagctgaacagactgacgtctgccgacacggccgtctattactgtgcgagagataatattatcgacccctggggccagggaaccttggtcaccgtctcctcag

SEQ ID NO: 283 - DNA, 369 bp, Artificial; identical to SEQ ID NO: 271
  DNA sequence coding for an IgE variable region

SEQ ID NO: 284 - DNA, 260 bp, Artificial
  DNA sequence coding for an IgE variable region
actcccaggtgcagctgctcgagtctggggctgaggtgaagaagcctggggcctcagtgaaggtctcctggaaagtcttctagatacaccttcaacgactacatataaacggggtgcgacaggcccctggataagggcctgagtggctgggacaagagattactttaattcagggagtttttagttcgcccgttgttgatacttccgctaataaaacatggacgtttggggcaaagggaccgcggtcaccgtctccgcag

SEQ ID NO: 285 - DNA, 381 bp, Artificial
  DNA sequence coding for an IgE variable region
gtcctgtcccaggtgcagctgctcgagtcgggcccaggactggtgaagccttcgcagaccctgtccctcacctgcactgtctctggtggctccatcggcagtaaactgacttctgggactggatccgccagcccccagggaagggactggagtggattgggagtatcgattacagtgggttcacctacttcaatccgtccctcaagagtcgaatcaccatctccgaagacacgtccaagaaccagttctccctgaaactgacctctgtgaccgccgcagacacggctgtctattactgtgcgagacatcgggacggtggtaaagtctgggagtactactttgacttctggggccagggaaccctggtcaccgtctcctcag

SEQ ID NO: 286 - DNA, 352 bp, Artificial
  DNA sequence coding for an IgE variable region
gtgcagctgctcgagtcgggcccaggactggtgaagccttcggggaccctgtccctcacctgcgctgtctctggtgactccatcagcagtggtaactggtggaattgggtccgccagcccccagggaaggggctggagtggattgggaagatctatcatagtgggagcatcaactacagcccgtccctcaagagtcgcgtcacgttctcagtagacaagtccaagaaccagttctccctggagttgaggtctgtgaccgccgcggacacggccatatattactgtgctggtggccgttacgccagctcccttgaccactggggccagggagccctggtcaccgtctcctcag

SEQ ID NO: 287 - DNA, 338 bp, Artificial; identical to SEQ ID NO: 280
  DNA sequence coding for an IgE variable region

SEQ ID NO: 288 - DNA, 338 bp, Artificial
  DNA sequence coding for an IgE variable region
cccaggactggtgaagccttcggagaccctgtctctcaactgcactgtctctggtgcttccgtcagcagtggcagttatcactggagttggatccggcagcccccagggaagggactggagtgggttgcatatatctattccagggggggcaccaattacaacccctcccctcaagagtcgagtcaccatatcagtggaacgtccaagaaccagttgtccctgaagctgagctctgtgaccgctgcggacacggccatatattactgtgtgagaacaggaatgataataagatacatggacgtctggggcaaagggaccacggtcaccgtctcctcag

SEQ ID NO: 289 - DNA, 393 bp, Artificial
  DNA sequence coding for an IgE variable region
gtcctgtcccagtgcagctgctcgagtcgggcccaggactggtgaagccttcacagaccctgtccctcacctgcactgtctctgctggctccatcagcggtagtggttactactggacctggatccgccagcacccagggaagggcctggagtggattggctacatctattacagtgggagcacctactacaacccgtcgctcaagagtcgagttaccatatcagcagatacgtctaagaaccagttctccctgaggttgagctctgtgactgccgcggacacggccgtgtattactgtgcgagaggcccgtattactatggtccagggagtcatcggtactactactacatggacgtctggggcaaagggaccacggtcaccgtctcctcag

SEQ ID NO: 290 - DNA, 382 bp, Artificial; identical to SEQ ID NO: 277
  DNA sequence coding for an IgE variable region

SEQ ID NO: 291 - DNA, 382 bp, Artificial
  DNA sequence coding for an IgE variable region
gtcctgtcccaggtgcagctgctcgagtcgggcccaggactggtgaagccttcgcagaccctgtccctcacctgcactgtctctggtggctccatcggcagtaatactgacttctgggactggatccgccagcccccagggaagggactggagtggattgggagtatcgattacagtgggcgcacctacttcaatccgtccctcaagagtcgaatcaccatatccgtagacacgtccaagacccagttctccctgaaactgacctctgtgaccgccgcagacacggctgtctattactgtgcgagacatcgggacggtggtaaagtctgggagtactactttgacttctggggccagggaaccctggtcaccgtctcctcag

SEQ ID NO: 292 - DNA, 364 bp, Artificial
  DNA sequence coding for an IgE variable region
gtcctgtcccaggtgcagctgctcgagtcgggcccaggactggtgaagccttcggggaccctgtccctcacctgcgctgtctctggtgactccatcagcagtggtaactggtggaattgggtccgccagcccccagggaaggggctggagtggattgggaagatctatcatagtgggagcatcaactacagcccgtcccccaagagtcgcgtcacgttctcagtagacaagtccaagaaccagttctccctggagttgaggtctgtgaccgccgcggacacggccatatattactgtgctggtggccgttacgccagctcccttgaccactggggccagggagccctggtcaccgtctcctcag

SEQ ID NO: 293 - DNA, 394 bp, Artificial
  DNA sequence coding for an IgE variable region
gtcctgtcccaggtgcagctgctcgagtcgggcccaggactggtgaagccttcacagaccctgtccctcacctgcactgtctctgctggctccatcagcggtagtggttactactggacctggatccgccagcacccagggaagggcctggagtggattggctacatctattacagtgggagcaccttactacaacccgtcgctcaagagtcgagtaccatatcagcagatacgtctaagaaccagttctccctgaggttgagctctgtgactgccgcggacacggccgtgtattactgtgcgagaggcccgtattactatggttcagggagtcatcggtactactactacatggacgtctggggcaaagggaccacggtcaccgtctcctcag

SEQ ID NO: 294 - DNA, 370 bp, Artificial
  DNA sequence coding for an IgE variable region
gtcctgtcccaggtgcagctgctcgagtcgggcccaggactggtgaagccttcggagaccctgtctctcaactgcactgtctctggtgcttccgtcagcagtggcagttatcactggagttggatccggcagcccccagggaaggggactggagtgggttgcatatatctattccgggggggcaccaattacaacccctccctcaagagtcgagtcaccatatcagtggacacgtccaagaaccagttgtccctgaagctgagctctgtgaccgctgcggacacggccatatattactgtgtgagaacaggaatgataataagatacatggacgtctggggcaaagggaccacggtcaccgtctcctcag

SEQ ID NO: 295 - DNA, 350 bp, Artificial
  DNA sequence coding for an IgE variable region
cccaggactggtgaagccttcggagaccctgtccctcacctgcactgtctctggtgcctccatcagttcaagtcgtgatttctgggactggctacgccagcccccagggaagggactggaatggattgggagtgtctatgacagcgggaccacctacctcaatccgtccctcaagagccgaatcaccatatccgtagacacgtccaagaaccagttctccctaaagttgagatctgtgaccgcctcagacacggctgtctattactgtgcgagacatcgggacgctgggaaaatctgggaatacttctttgacttctggggccggggaaccctggtcgccgtctcctcag

SEQ ID NO: 296 - DNA, 341 bp, Artificial
  DNA sequence coding for an IgE variable region
cccaggactggtgaagccttcacagagcctgtctctcacctgcattgtctctggggactccatcgacagtggtggttactactggtcctggatccgccagcacccagggaagggcctggagtggattgcgtacatgtatcacagtgggagcgcctactacaatccgtccctcaggagtcgaatttcctcatcagtagacacgtcgaagaaccagttctccctgaggctgacctctgtgactgccgcggacacggccgtgtattattgtgcgagagggctcggttttggagtggcccccctattattttggggccagggaatcctggtcaccgtctcctcag

SEQ ID NO: 297 - DNA, 350 bp, Artificial
  DNA sequence coding for an IgE variable region
cccaggactggtgaagccttcggagaccctgtccctcacctgcactgtctctggtgcctccatcagttcaagtcgtgatttctgggactggctacgccagcccccagggaagggactggaatggattgggagtgtctatgacagcgggaccacctacctcaatccgtccctcaagagccgaatcaccatatccgtagacacgtccaagaaccagttctccctaaagttgagatctgtgaccgcctcagacacggctgtctattactgtgcgagacatcgggacgctgggaaaatctgggaatacttctttgacttctggggccggggaaccctggtcaccgtctcctcag

SEQ ID NO: 298 - DNA, 347 bp, Artificial
  DNA sequence coding for an IgE variable region
aggaggcttgatccagcctggggggtccctgagactctcctgtgcagcctccgggttcaccgtcagtggcaacttcctgagctgggtccgccaggctccagggaaggggctggagtgggtctcagttctgtatagcagtgggaccacatactacgcagattccgtgaagggccgcttcaccatctccagagacaattccaagaacacgctgtatcttcaaatgaacagcctgagagtcgaggacacggccgtgtattattgtgcggccccggtatccaggctggaccactaccactactactacatggacatctggggcaaagggaccacggtcaccgtctcctcag